SEQUENCE LISTING (499 sequences)

SEQ ID NO: 1 - protein, 17 aa, Homo sapiens
KQQMAREYREKIETELR

SEQ ID NO: 2 - protein, 12 aa, Homo sapiens
NDDGWYEGVCNR

SEQ ID NO: 3 - protein, 10 aa, Homo sapiens
CLKDEDPYVR

SEQ ID NO: 4 - protein, 13 aa, Homo sapiens
NVEGQDMLYQSLK

SEQ ID NO: 5 - protein, 36 aa, Homo sapiens
VISANPYLGGTSNGYAHPSGTALHYDDVPCINGSLK

SEQ ID NO: 6 - protein, 15 aa, Homo sapiens
WEAGIYANQEEEDNE

SEQ ID NO: 7 - protein, 15 aa, Homo sapiens
LFEDDEHEKEQYCIR

SEQ ID NO: 8 - protein, 32 aa, Homo sapiens
SPGALETPSAAGSQGNTASQGKEGPYSEPSKR

SEQ ID NO: 9 - protein, 27 aa, Homo sapiens
SDPFVPPAASSEPLSTPWNELNYVGGR

SEQ ID NO: 10 - protein, 27 aa, Homo sapiens
CEVNAEDKENSGDYISENEDPELQDYR

SEQ ID NO: 11 - protein, 9 aa, Homo sapiens
SLENQTYFK

SEQ ID NO: 12 - protein, 19 aa, Homo sapiens
RILYQNLNEPTTWSLTSDR

SEQ ID NO: 13 - protein, 26 aa, Homo sapiens
HDVSAHHDLNIDQSQCNEMYINSSQR

SEQ ID NO: 14 - protein, 25 aa, Homo sapiens
GQQQQQQQQGAVGHGYYMAGGTSQK

SEQ ID NO: 15 - protein, 13 aa, Homo sapiens
LRCYCMTDDKVDK

SEQ ID NO: 16 - protein, 7 aa, Homo sapiens
PYSVGFR

SEQ ID NO: 17 - protein, 15 aa, Homo sapiens
ELVNYGANVNAQSQK

SEQ ID NO: 18 - protein, 13 aa, Homo sapiens
VGYLGAMLLLDER

SEQ ID NO: 19 - protein, 11 aa, Homo sapiens
ALDLLYGMVSK

SEQ ID NO: 20 - protein, 17 aa, Homo sapiens
SVVSQSVCDYFFEAQEK

SEQ ID NO: 21 - protein, 21 aa, Homo sapiens
EGYQDYYPEEANGNTGASPYR

SEQ ID NO: 22 - protein, 22 aa, Homo sapiens
THCDCNVDWCLYEIYPELQIER

SEQ ID NO: 23 - protein, 9 aa, Homo sapiens
ASGIYYVPK

SEQ ID NO: 24 - protein, 10 aa, Homo sapiens
AEFNYSVGFK

SEQ ID NO: 25 - protein, 13 aa, Homo sapiens
SDDHPDVVYETMR

SEQ ID NO: 26 - protein, 26 aa, Homo sapiens
DFVDHLDKVDPVDGVVLVDPDYLKDR

SEQ ID NO: 27 - protein, 32 aa, Homo sapiens
TRSQRKVGGGSAQPCDSIVVAYYFCGEPIPYR

SEQ ID NO: 28 - protein, 32 aa, Homo sapiens; identical to SEQ ID NO: 27

SEQ ID NO: 29 - protein, 32 aa, Homo sapiens; identical to SEQ ID NO: 27

SEQ ID NO: 30 - protein, 10 aa, Homo sapiens
VENEYTISVK

SEQ ID NO: 31 - protein, 11 aa, Homo sapiens
LNHNLYEVMSK

SEQ ID NO: 32 - protein, 12 aa, Homo sapiens
TGHGYVYEYPSR

SEQ ID NO: 33 - protein, 20 aa, Homo sapiens
DTCYSPKPSVYLSTPSSASK

SEQ ID NO: 34 - protein, 9 aa, Homo sapiens
ASTYGVAVR

SEQ ID NO: 35 - protein, 20 aa, Homo sapiens
GKHGNRNSNSYGIPEPAHAY

SEQ ID NO: 36 - protein, 18 aa, Homo sapiens
DSSTCPGDYVLSVSENSR

SEQ ID NO: 37 - protein, 15 aa, Homo sapiens
IHYLDTTTLIEPAPR

SEQ ID NO: 38 - protein, 11 aa, Homo sapiens
EMVSQYLYTSK

SEQ ID NO: 39 - protein, 12 aa, Homo sapiens
HHTSSVYSISER

SEQ ID NO: 40 - protein, 19 aa, Homo sapiens
NKPVPDQIINFYKSNYVQR

SEQ ID NO: 41 - protein, 14 aa, Homo sapiens
SLYDSPQEPRGEAW

SEQ ID NO: 42 - protein, 29 aa, Homo sapiens
QLPNSYNLEKITVNSVSRDNSKYHCTAYR

SEQ ID NO: 43 - protein, 29 aa, Homo sapiens
QLPNSYNLEKITVNSVSRDNSKYHCTAYR

SEQ ID NO: 44 - protein, 20 aa, Homo sapiens
DYEPPSPSPAPGAPPPPPQR

SEQ ID NO: 45 - protein, 8 aa, Homo sapiens
VEQQPDYR

SEQ ID NO: 46 - protein, 10 aa, Homo sapiens
MSEPPVYCNL

SEQ ID NO: 47 - protein, 14 aa, Homo sapiens
AMENQYSPTPGTDC

SEQ ID NO: 48 - protein, 29 aa, Homo sapiens
SPISTGQPTNQSMDDTREDIYVNYPTFSR

SEQ ID NO: 49 - protein, 17 aa, Homo sapiens
DSSCGTGYELTEDNSCK

SEQ ID NO: 50 - protein, 15 aa, Homo sapiens
LEGEVTPNSLSTSYK

SEQ ID NO: 51 - protein, 20 aa, Homo sapiens
SRSAPPNLWAAQRYGRELRR

SEQ ID NO: 52 - protein, 22 aa, Homo sapiens
SHLMSLYSACSSEVPHGPVDQK

SEQ ID NO: 53 - protein, 24 aa, Homo sapiens
AGGSHQEQPPYPSYNSNYWNSTAR

SEQ ID NO: 54 - protein, 13 aa, Homo sapiens
SGYGPSDGPSYGR

SEQ ID NO: 55 - protein, 27 aa, Homo sapiens
SVPQSGPTVRPQEDAWASPGAYGMGGR

SEQ ID NO: 56 - protein, 15 aa, Homo sapiens
DSSYPYSQSDQSMNR

SEQ ID NO: 57 - protein, 13 aa, Homo sapiens
SGYGPSDGPSYGR

SEQ ID NO: 58 - protein, 16 aa, Homo sapiens
GAGAFGYFEVTHDITK

SEQ ID NO: 59 - protein, 11 aa, Homo sapiens
AKYDTPYIIWR

SEQ ID NO: 60 - protein, 15 aa, Homo sapiens
TPVLFDIYEIKEAIK

SEQ ID NO: 61 - protein, 12 aa, Homo sapiens
DAQELYAAGENR

SEQ ID NO: 62 - protein, 9 aa, Homo sapiens
DLYDAGVKR

SEQ ID NO: 63 - protein, 15 aa, Homo sapiens
SIPAYLAETLYYAMK

SEQ ID NO: 64 - protein, 11 aa, Homo sapiens
NFATSLYSMIK

SEQ ID NO: 65 - protein, 12 aa, Homo sapiens
NKPLFFADKLYK

SEQ ID NO: 66 - protein, 15 aa, Homo sapiens
LIVGLMRPPAYCDAK

SEQ ID NO: 67 - protein, 19 aa, Homo sapiens
SGYIDEHELDALLKDLYEK

SEQ ID NO: 68 - protein, 14 aa, Homo sapiens
FYALSASFEPFSNK

SEQ ID NO: 69 - protein, 19 aa, Homo sapiens
LHTLEEEKEELAQYQKWDK

SEQ ID NO: 70 - protein, 14 aa, Homo sapiens
LFYHIVDSDEVSTK

SEQ ID NO: 71 - protein, 12 aa, Homo sapiens
GALTGGYYDTRK

SEQ ID NO: 72 - protein, 23 aa, Homo sapiens
TPCNAGTFSQPEKVYTLSVSGDR

SEQ ID NO: 73 - protein, 14 aa, Homo sapiens
VAVEYLDPSPEVQK

SEQ ID NO: 74 - protein, 7 aa, Homo sapiens
AALYFQR

SEQ ID NO: 75 - protein, 14 aa, Homo sapiens
LEYFSCDHQELLQR

SEQ ID NO: 76 - protein, 25 aa, Homo sapiens
ISHIPENFDDYVDINEDEDCYSDER

SEQ ID NO: 77 - protein, 25 aa, Homo sapiens
ISHIPENFDDYVDINEDEDCYSDER

SEQ ID NO: 78 - protein, 11 aa, Homo sapiens
EKDVSEYFYEK

SEQ ID NO: 79 - protein, 9 aa, Homo sapiens
NQYQALKPR

SEQ ID NO: 80 - protein, 15 aa, Homo sapiens
VLTTGYWPTQSATPK

SEQ ID NO: 81 - protein, 24 aa, Homo sapiens
VHTVEDYQAIVDAEWNILYDKLEK

SEQ ID NO: 82 - protein, 19 aa, Homo sapiens
KRVEDAYILTCNVSLEYEK

SEQ ID NO: 83 - protein, 12 aa, Homo sapiens
SLEYEKTEVNSG

SEQ ID NO: 84 - protein, 11 aa, Homo sapiens
EKKLYANMFER

SEQ ID NO: 85 - protein, 17 aa, Homo sapiens
EAAGEGPALYEDPPDQK

SEQ ID NO: 86 - protein, 25 aa, Homo sapiens
ISMQDPKMQVYKDEQVVVIKDKYPK

SEQ ID NO: 87 - protein, 17 aa, Homo sapiens
ARYHWLVLPWTSISSLK

SEQ ID NO: 88 - protein, 10 aa, Homo sapiens
NDMTYNYANR

SEQ ID NO: 89 - protein, 10 aa, Homo sapiens
NDMTYNYANR

SEQ ID NO: 90 - protein, 22 aa, Homo sapiens
TLVQNNCLTRPNIYLIPDIDLK

SEQ ID NO: 91 - protein, 14 aa, Homo sapiens
SSNAYDPSQMCAEK

SEQ ID NO: 92 - protein, 12 aa, Homo sapiens
YRHIKPVSRNSR

SEQ ID NO: 93 - protein, 13 aa, Homo sapiens
TKKPDLQIYQPGR

SEQ ID NO: 94 - protein, 12 aa, Homo sapiens
FQNSDNPYYYPR

SEQ ID NO: 95 - protein, 14 aa, Homo sapiens
YASNLPGSLLKEQK

SEQ ID NO: 96 - protein, 16 aa, Homo sapiens
VDNEILDYKDLAAIPK

SEQ ID NO: 97 - protein, 25 aa, Homo sapiens
NGLHRPVSTDFAQYNSYGDVSGGVR

SEQ ID NO: 98 - protein, 14 aa, Homo sapiens
DYQTLPDGHMPAMR

SEQ ID NO: 99 - protein, 26 aa, Homo sapiens
IVQTYHVNMAGTNPYTTITPQEINGK

SEQ ID NO: 100 - protein, 21 aa, Homo sapiens
ISIEMNGTLEDQLSHLKQYER

SEQ ID NO: 101 - protein, 15 aa, Homo sapiens
SMLEVNYPMENGIVR

SEQ ID NO: 102 - protein, 23 aa, Homo sapiens
NWDDMKHLWDYTFGPEKLNIDTR

SEQ ID NO: 103 - protein, 24 aa, Homo sapiens
YLRAEPEDHYFLLTEPPLNTPENR

SEQ ID NO: 104 - protein, 14 aa, Homo sapiens
LPACVVDCGTGYTK

SEQ ID NO: 105 - protein, 16 aa, Homo sapiens
ETKDTDIVDEAIYYFK

SEQ ID NO: 106 - protein, 17 aa, Homo sapiens
IQVHYYEDGNVQLVSHK

SEQ ID NO: 107 - protein, 21 aa, Homo sapiens
PLDHAQPPSSLVIDKESEVYK

SEQ ID NO: 108 - protein, 11 aa, Homo sapiens
MIYASSKDAIK

SEQ ID NO: 109 - protein, 24 aa, Homo sapiens
YFEITDESPYVHYLNTFSSKEPQR

SEQ ID NO: 110 - protein, 16 aa, Homo sapiens
REQRYQEQGGEASPQR

SEQ ID NO: 111 - protein, 11 aa, Homo sapiens
MIYASSKDAIK

SEQ ID NO: 112 - protein, 14 aa, Homo sapiens
TLDDIVGRYEDLSK

SEQ ID NO: 113 - protein, 8 aa, Homo sapiens
IFEYETQR

SEQ ID NO: 114 - protein, 21 aa, Homo sapiens
LSPPSSSAASSYSFSDLNSTR

SEQ ID NO: 115 - protein, 15 aa, Homo sapiens
VSLLDDTVYECVVEK

SEQ ID NO: 116 - protein, 11 aa, Homo sapiens
TITYEAAQTVK

SEQ ID NO: 117 - protein, 11 aa, Homo sapiens
YSGKTEYQTTK

SEQ ID NO: 118 - protein, 12 aa, Homo sapiens
ASVMVYDDTSKK

SEQ ID NO: 119 - protein, 13 aa, Homo sapiens
INIYHNTASNTFR

SEQ ID NO: 120 - protein, 26 aa, Homo sapiens
EGILSDEIYCPPETAVLLGSYAVQAK

SEQ ID NO: 121 - protein, 22 aa, Homo sapiens
THTGKYWTLTATGGVQSTASSK

SEQ ID NO: 122 - protein, 11 aa, Homo sapiens
NASCYFDIEWR

SEQ ID NO: 123 - protein, 13 aa, Homo sapiens
CSNEKGYFAVTEK

SEQ ID NO: 124 - protein, 16 aa, Homo sapiens
VGLGLGYLELPQINYK

SEQ ID NO: 125 - protein, 15 aa, Homo sapiens
NSEGWEQNGLYEFFR

SEQ ID NO: 126 - protein, 15 aa, Homo sapiens
GVGVALDDPYENYRR

SEQ ID NO: 127 - protein, 15 aa, Homo sapiens
GVGVALDDPYENYRR

SEQ ID NO: 128 - protein, 13 aa, Homo sapiens
HYLSTKNGAGLSK

SEQ ID NO: 129 - protein, 22 aa, Homo sapiens
DNFWEMGDTGPCGPCSEIHYDR

SEQ ID NO: 130 - protein, 13 aa, Homo sapiens
AVYTQDCPLAAAK

SEQ ID NO: 131 - protein, 16 aa, Homo sapiens
TRADKEGDYYVLNGSK

SEQ ID NO: 132 - protein, 26 aa, Homo sapiens
QASVGAGIPYSVPAWSCQMICGSGLK

SEQ ID NO: 133 - protein, 7 aa, Homo sapiens
LKQGLYR

SEQ ID NO: 134 - protein, 15 aa, Homo sapiens
EAYPEEAYIADLDAK

SEQ ID NO: 135 - protein, 15 aa, Homo sapiens
EAYPEEAYIADLDAK

SEQ ID NO: 136 - protein, 11 aa, Homo sapiens
RGGPNYQEGLR

SEQ ID NO: 137 - protein, 10 aa, Homo sapiens
LTYGTMVFVR

SEQ ID NO: 138 - protein, 14 aa, Homo sapiens
YDGNVYENLFEWAK

SEQ ID NO: 139 - protein, 11 aa, Homo sapiens
TEGGYYQITGR

SEQ ID NO: 140 - protein, 11 aa, Homo sapiens
DQDGYYWITGR

SEQ ID NO: 141 - protein, 11 aa, Homo sapiens
DQDGYYWITGR

SEQ ID NO: 142 - protein, 21 aa, Homo sapiens
VELHVHLDGSIKPETILYYGR

SEQ ID NO: 143 - protein, 11 aa, Homo sapiens
STLDTDYQMTK

SEQ ID NO: 144 - protein, 22 aa, Homo sapiens
ELLDLLYKAYGMPPSASAGQNL

SEQ ID NO: 145 - protein, 12 aa, Homo sapiens
FDYYMPAIAGCR

SEQ ID NO: 146 - protein, 37 aa, Homo sapiens
SHMHLASAFAGIGFGNAGVHLCHGMSYPISGLVKMYK

SEQ ID NO: 147 - protein, 37 aa, Homo sapiens; identical to SEQ ID NO: 146

SEQ ID NO: 148 - protein, 10 aa, Homo sapiens
KGIGPVYSSK

SEQ ID NO: 149 - protein, 16 aa, Homo sapiens
DGVYFLYEALHGPPKK

SEQ ID NO: 150 - protein, 16 aa, Homo sapiens
DGVYFLYEALHGPPKK

SEQ ID NO: 151 - protein, 15 aa, Homo sapiens
GLEVTAYSPLGSSDR

SEQ ID NO: 152 - protein, 26 aa, Homo sapiens
HIDCAAIYGNEPEIGEALKEDVGPGK

SEQ ID NO: 153 - protein, 15 aa, Homo sapiens
ANNTFYGLSAGVFTK

SEQ ID NO: 154 - protein, 14 aa, Homo sapiens
ELGEYGFHEYTEVK

SEQ ID NO: 155 - protein, 25 aa, Homo sapiens
YYAGWADKIHGMTIPVDGDYFTFTR

SEQ ID NO: 156 - protein, 18 aa, Homo sapiens
SAPYEFPEESPIEQLEER

SEQ ID NO: 157 - protein, 10 aa, Homo sapiens
IDYIAGLDSR

SEQ ID NO: 158 - protein, 13 aa, Homo sapiens
YYADGEDAYAMKR

SEQ ID NO: 159 - protein, 13 aa, Homo sapiens
FELSCYSLAPQIK

SEQ ID NO: 160 - protein, 19 aa, Homo sapiens
FAELVYTGFWHSPECEFVR

SEQ ID NO: 161 - protein, 12 aa, Homo sapiens
LAGDKANYWWLR

SEQ ID NO: 162 - protein, 13 aa, Homo sapiens
WHVARKPLDVYKK

SEQ ID NO: 163 - protein, 13 aa, Homo sapiens
WKCYDALRKMINY

SEQ ID NO: 164 - protein, 13 aa, Homo sapiens
WKCYDALRKMINY

SEQ ID NO: 165 - protein, 12 aa, Homo sapiens
EGWPNSPYGVSK

SEQ ID NO: 166 - protein, 15 aa, Homo sapiens
GINTLVTYDMVPEPK

SEQ ID NO: 167 - protein, 13 aa, Homo sapiens
LTSDSTVYDYAGK

SEQ ID NO: 168 - protein, 29 aa, Homo sapiens
EIQYGYRRVNPMHGVEYILDLLLLYKRHK

SEQ ID NO: 169 - protein, 9 aa, Homo sapiens
LHLYNSLTR

SEQ ID NO: 170 - protein, 13 aa, Homo sapiens
NNAYLAQSPQLYK

SEQ ID NO: 171 - protein, 19 aa, Homo sapiens
YHYELLNYIDLPVLAIHGK

SEQ ID NO: 172 - protein, 19 aa, Homo sapiens
YHYELLNYIDLPVLAIHGK

SEQ ID NO: 173 - protein, 19 aa, Homo sapiens
YHYELLNYIDLPVLAIHGK

SEQ ID NO: 174 - protein, 10 aa, Homo sapiens
GANLKDYYSR

SEQ ID NO: 175 - protein, 16 aa, Homo sapiens
QLYHLGVVEAYSGLTK

SEQ ID NO: 176 - protein, 8 aa, Homo sapiens
DFVNYLVR

SEQ ID NO: 177 - protein, 13 aa, Homo sapiens
TEMQDNTYPEILR

SEQ ID NO: 178 - protein, 7 aa, Homo sapiens
FQDGYYS

SEQ ID NO: 179 - protein, 14 aa, Homo sapiens
ASLLDDYQLPEILR

SEQ ID NO: 180 - protein, 18 aa, Homo sapiens
HAITTYLFCTYPDAHEGR

SEQ ID NO: 181 - protein, 18 aa, Homo sapiens
HYTGPGDQTVNPQNGFVR

SEQ ID NO: 182 - protein, 19 aa, Homo sapiens
HATGNYIIIMDADLSHHPK

SEQ ID NO: 183 - protein, 23 aa, Homo sapiens
NLHDALSGHTSNNIYEAVKYSER

SEQ ID NO: 184 - protein, 9 aa, Homo sapiens
GTGLYELWK

SEQ ID NO: 185 - protein, 13 aa, Homo sapiens
GNPTVEVDLYTAK

SEQ ID NO: 186 - protein, 8 aa, Homo sapiens
DNYSPGWK

SEQ ID NO: 187 - protein, 21 aa, Homo sapiens
TTEYHDRDEQFYWIIEALGIR

SEQ ID NO: 188 - protein, 21 aa, Homo sapiens
TTEYHDRDEQFYWIIEALGIR

SEQ ID NO: 189 - protein, 15 aa, Homo sapiens
SLYDEVAAQGEVVRK

SEQ ID NO: 190 - protein, 13 aa, Homo sapiens
FANHSVNPNCYAK

SEQ ID NO: 191 - protein, 13 aa, Homo sapiens
VEYVIGRYPQENK

SEQ ID NO: 192 - protein, 13 aa, Homo sapiens
VEYVIGRYPQENK

SEQ ID NO: 193 - protein, 13 aa, Homo sapiens
VNLQMVYDSPLCR

SEQ ID NO: 194 - protein, 11 aa, Homo sapiens
AFDTAGNGYCR

SEQ ID NO: 195 - protein, 17 aa, Homo sapiens
HTPVDHPDYPLLQDALR

SEQ ID NO: 196 - protein, 15 aa, Homo sapiens
ATYSGFLYCSPVSNK

SEQ ID NO: 197 - protein, 26 aa, Homo sapiens
KKYNDDSDDSYFTSSSSYFDEPVELR

SEQ ID NO: 198 - protein, 17 aa, Homo sapiens
RKPDYEPVENTDEAQKK

SEQ ID NO: 199 - protein, 10 aa, Homo sapiens
AISSDMYFGR

SEQ ID NO: 200 - protein, 34 aa, Homo sapiens
VSGQPQSVTASSDKAFEDWLNDDLGSYQGAQGNR

SEQ ID NO: 201 - protein, 14 aa, Homo sapiens
SLVDTVYALKDEVR

SEQ ID NO: 202 - protein, 9 aa, Homo sapiens
SFDLTTPYR

SEQ ID NO: 203 - protein, 16 aa, Homo sapiens
WCVLGDGVLSYFENER

SEQ ID NO: 204 - protein, 21 aa, Homo sapiens
VAVSLGTPIMKPEWIYKAWER

SEQ ID NO: 205 - protein, 13 aa, Homo sapiens
SIEEREEEYQRVR

SEQ ID NO: 206 - protein, 13 aa, Homo sapiens
CGEDDETIPSEYR

SEQ ID NO: 207 - protein, 13 aa, Homo sapiens
APSVPAAEPEYPK

SEQ ID NO: 208 - protein, 16 aa, Homo sapiens
LQAYHTQTTPLIEYYR

SEQ ID NO: 209 - protein, 25 aa, Homo sapiens
DQECVNSCASSETSYGTNESSLNEK

SEQ ID NO: 210 - protein, 14 aa, Homo sapiens
LVRPPVQVYGIEGR

SEQ ID NO: 211 - protein, 11 aa, Homo sapiens
YNMPFKAQIQK

SEQ ID NO: 212 - protein, 11 aa, Homo sapiens
MKELEDNLLYK

SEQ ID NO: 213 - protein, 17 aa, Homo sapiens
YQVGVHYELTEEEKFYR

SEQ ID NO: 214 - protein, 26 aa, Homo sapiens
TKPVTGNLRPEEALQALTIYEGKFGR

SEQ ID NO: 215 - protein, 19 aa, Homo sapiens
CVLNWFGDWSTEALYQVGK

SEQ ID NO: 216 - protein, 15 aa, Homo sapiens
NYMSNPSYNYEIVNR

SEQ ID NO: 217 - protein, 17 aa, Homo sapiens
LSHEGPGSELPAGALYR

SEQ ID NO: 218 - protein, 14 aa, Homo sapiens
AKEEQQDDTVYMGK

SEQ ID NO: 219 - protein, 10 aa, Homo sapiens
VHQLYETIQR

SEQ ID NO: 220 - protein, 11 aa, Homo sapiens
KAYYLACGFCR

SEQ ID NO: 221 - protein, 11 aa, Homo sapiens
KAYYLACGFCR

SEQ ID NO: 222 - protein, 19 aa, Homo sapiens
GLEEITVHNKDEVYQILEK

SEQ ID NO: 223 - protein, 11 aa, Homo sapiens
NGVYISEENFR

SEQ ID NO: 224 - protein, 22 aa, Homo sapiens
QITKEDFATFDYILCMDESNLR

SEQ ID NO: 225 - protein, 17 aa, Homo sapiens
LRDDGCQAYYLQGGFNK

SEQ ID NO: 226 - protein, 26 aa, Homo sapiens
LKKPDQAIKGDQFVFYEDWGENMVSK

SEQ ID NO: 227 - protein, 23 aa, Homo sapiens
GSTAGGCRNYPNTFWMNPQYLIK

SEQ ID NO: 228 - protein, 12 aa, Homo sapiens
KWNTTLYEGTWR

SEQ ID NO: 229 - protein, 21 aa, Homo sapiens
GSTAGGCRNYPATFWVNPQFK

SEQ ID NO: 230 - protein, 15 aa, Homo sapiens
SQGQHWYSSSDKNCK

SEQ ID NO: 231 - protein, 12 aa, Homo sapiens
SEYSVHSSLASK

SEQ ID NO: 232 - protein, 9 aa, Homo sapiens
HINEVYYAK

SEQ ID NO: 233 - protein, 16 aa, Homo sapiens
VYNDGYDDDNYDYIVK

SEQ ID NO: 234 - protein, 29 aa, Homo sapiens
REQGGSGLGSGSSGGGGSTSGLGSGYIGR

SEQ ID NO: 235 - protein, 27 aa, Homo sapiens
TGHSFWEIYKMTTDPMLAYLQPFRTSR

SEQ ID NO: 236 - protein, 27 aa, Homo sapiens
TGHSFWEIYKMTTDPMLAYLQPFRTSR

SEQ ID NO: 237 - protein, 15 aa, Homo sapiens
SIGEPDSLYGCGGGK

SEQ ID NO: 238 - protein, 36 aa, Homo sapiens
DNLIDANGGSRPPWPPLEYQPYQSIYVGGMMEGEGK

SEQ ID NO: 239 - protein, 21 aa, Homo sapiens
HQDGLPYIDDSPSSSPHLSSK

SEQ ID NO: 240 - protein, 29 aa, Homo sapiens
KWVLSGILASEETYLSHLEALLLPMKPLK

SEQ ID NO: 241 - protein, 9 aa, Homo sapiens
YSDMSSVYR

SEQ ID NO: 242 - protein, 9 aa, Homo sapiens
YSDMSSVYR

SEQ ID NO: 243 - protein, 14 aa, Homo sapiens
GPIEQVYQEIAILK

SEQ ID NO: 244 - protein, 13 aa, Homo sapiens
IGEGTYGVVYKAK

SEQ ID NO: 245 - protein, 35 aa, Homo sapiens
YKLVRKIGSGSFGDIYLAINITNGEEVALKLESQK

SEQ ID NO: 246 - protein, 35 aa, Homo sapiens; identical to SEQ ID NO: 245

SEQ ID NO: 247 - protein, 11 aa, Homo sapiens
ARVYAEVNSLR

SEQ ID NO: 248 - protein, 13 aa, Homo sapiens
ISLNSLCYGDMDK

SEQ ID NO: 249 - protein, 25 aa, Homo sapiens
CSTCLPATCGLPAEYATHFTEAFCR

SEQ ID NO: 250 - protein, 9 aa, Homo sapiens
SIGEYDVLR

SEQ ID NO: 251 - protein, 12 aa, Homo sapiens
SIYIDRHVMQPY

SEQ ID NO: 252 - protein, 9 aa, Homo sapiens
SYVAWDREK

SEQ ID NO: 253 - protein, 26 aa, Homo sapiens
YTNLSYIGEGAYGMVCSAYDNVNKVR

SEQ ID NO: 254 - protein, 26 aa, Homo sapiens
YTNLSYIGEGAYGMVCSAYDNVNKVR

SEQ ID NO: 255 - protein, 10 aa, Homo sapiens
LLLSPNNYTK

SEQ ID NO: 256 - protein, 33 aa, Homo sapiens
ENLLAGPSENDPNLFVALYDFVASGDNTLSITK

SEQ ID NO: 257 - protein, 27 aa, Homo sapiens
STGDPQGVIRHYVVCSTPQSQYYLAEK

SEQ ID NO: 258 - protein, 10 aa, Homo sapiens
LSYYEYDFER

SEQ ID NO: 259 - protein, 16 aa, Homo sapiens
MDAPDGCPPAVYEVMK

SEQ ID NO: 260 - protein, 11 aa, Homo sapiens
WTAPEALNYGR

SEQ ID NO: 261 - protein, 12 aa, Homo sapiens
KLDMGGYYITTR

SEQ ID NO: 262 - protein, 12 aa, Homo sapiens
KLDMGGYYITTR

SEQ ID NO: 263 - protein, 26 aa, Homo sapiens
HKNIINLLGACTQGGPLYVLVEYAAK

SEQ ID NO: 264 - protein, 22 aa, Homo sapiens
RPPGLDYSFDTCKPPEEQLTFK

SEQ ID NO: 265 - protein, 19 aa, Homo sapiens
DVHNLDYYKKTTNGRLPVK

SEQ ID NO: 266 - protein, 19 aa, Homo sapiens
DVHNLDYYKKTTNGRLPVK

SEQ ID NO: 267 - protein, 12 aa, Homo sapiens
EYDEPYVLLQNK

SEQ ID NO: 268 - protein, 12 aa, Homo sapiens
EYDEPYVLLQNK

SEQ ID NO: 269 - protein, 12 aa, Homo sapiens
YPYWPHQPIENL

SEQ ID NO: 270 - protein, 12 aa, Homo sapiens
YPYWPHQPIENL

SEQ ID NO: 271 - protein, 17 aa, Homo sapiens
LAALPENPPAIDWAYYK

SEQ ID NO: 272 - protein, 17 aa, Homo sapiens
ALDEYYDKHFTEFVPLR

SEQ ID NO: 273 - protein, 33 aa, Homo sapiens
HLDSSAAAIIDAPMDIPGLNLSQQEYYPYVYYK

SEQ ID NO: 274 - protein, 7 aa, Homo sapiens
IMEYYEK

SEQ ID NO: 275 - protein, 10 aa, Homo sapiens
LNEKNYELLK

SEQ ID NO: 276 - protein, 10 aa, Homo sapiens
GYADLISSGR

SEQ ID NO: 277 - protein, 12 aa, Homo sapiens
ATYVPSADDVQR

SEQ ID NO: 278 - protein, 27 aa, Homo sapiens
EREMKEMGYAATEQEISLREKLQEELK

SEQ ID NO: 279 - protein, 13 aa, Homo sapiens
SGYGPSDGPSYGR

SEQ ID NO: 280 - protein, 29 aa, Homo sapiens
LGEDPYYTENGGGQGYSSGPGTSPEAQGK

SEQ ID NO: 281 - protein, 17 aa, Homo sapiens
DKNSAACVVYEDMSHSR

SEQ ID NO: 282 - protein, 10 aa, Homo sapiens
TIYTYIMASR

SEQ ID NO: 283 - protein, 18 aa, Homo sapiens
ASTQDSKPPGTSSYEIVI

SEQ ID NO: 284 - protein, 12 aa, Homo sapiens
EVYSVDPYNPAS

SEQ ID NO: 285 - protein, 10 aa, Homo sapiens
AASADYLRPR

SEQ ID NO: 286 - protein, 12 aa, Homo sapiens
FKANDHGYDNFR

SEQ ID NO: 287 - protein, 10 aa, Homo sapiens
GENVVYSEVR

SEQ ID NO: 288 - protein, 16 aa, Homo sapiens
GPPPTDPYGRPPPYDR

SEQ ID NO: 289 - protein, 14 aa, Homo sapiens
GAAPNVVYTYTGKR

SEQ ID NO: 290 - protein, 15 aa, Homo sapiens
SIPAYLAETLYYAMK

SEQ ID NO: 291 - protein, 31 aa, Homo sapiens
TLYDHVDEVTCLAFHPTEQILASGSRDYTLK

SEQ ID NO: 292 - protein, 16 aa, Homo sapiens
LGMENDDTAVQYAIGR

SEQ ID NO: 293 - protein, 13 aa, Homo sapiens
FMDLYPCSASELK

SEQ ID NO: 294 - protein, 16 aa, Homo sapiens
ELFEPYGAVYQINVLR

SEQ ID NO: 295 - protein, 24 aa, Homo sapiens
QAFPAYPSSPFQVTTGYQLPVYNY

SEQ ID NO: 296 - protein, 24 aa, Homo sapiens
QAFPAYPSSPFQVTTGYQLPVYNY

SEQ ID NO: 297 - protein, 24 aa, Homo sapiens
QAFPAYPSSPFQVTTGYQLPVYNY

SEQ ID NO: 298 - protein, 20 aa, Homo sapiens
IDRIEESDQGPYAIILAPTR

SEQ ID NO: 299 - protein, 17 aa, Homo sapiens
VRPCVVYGGADIGQQIR

SEQ ID NO: 300 - protein, 25 aa, Homo sapiens
GFYDKDSSGWSSSKDKDAYSSFGSR

SEQ ID NO: 301 - protein, 15 aa, Homo sapiens
DRENYDRGYSSLLKR

SEQ ID NO: 302 - protein, 26 aa, Homo sapiens
GHNCPKPVLNFYEANFPANVMDVIAR

SEQ ID NO: 303 - protein, 14 aa, Homo sapiens
SQQSAGKEYVGIVR

SEQ ID NO: 304 - protein, 16 aa, Homo sapiens
NYILDQTNVYGSAQRR

SEQ ID NO: 305 - protein, 23 aa, Homo sapiens
VSYARPSSEVIKDANLYISGLPR

SEQ ID NO: 306 - protein, 16 aa, Homo sapiens
LDSYVNADHDLYCNTR

SEQ ID NO: 307 - protein, 13 aa, Homo sapiens
LMLSTSEYSQSPK

SEQ ID NO: 308 - protein, 12 aa, Homo sapiens
IQNMLGNYEEVK

SEQ ID NO: 309 - protein, 19 aa, Homo sapiens
LVVEEYYEVLDHCSSILNK

SEQ ID NO: 310 - protein, 12 aa, Homo sapiens
GQLNYPLPDFSK

SEQ ID NO: 311 - protein, 11 aa, Homo sapiens
TGYFSSTDLGR

SEQ ID NO: 312 - protein, 16 aa, Homo sapiens
VLSRPNAQELPSMYQR

SEQ ID NO: 313 - protein, 13 aa, Homo sapiens
LLLGAGAVAYGVR

SEQ ID NO: 314 - protein, 13 aa, Homo sapiens
IPWFQYPIIYDIR

SEQ ID NO: 315 - protein, 15 aa, Homo sapiens
LAKRIANSAGYVGDR

SEQ ID NO: 316 - protein, 16 aa, Homo sapiens
FNDSEGDDTEETEDYR

SEQ ID NO: 317 - protein, 10 aa, Homo sapiens
EYSGFAGVSR

SEQ ID NO: 318 - protein, 12 aa, Homo sapiens
KPAEYGYVSLGR

SEQ ID NO: 319 - protein, 16 aa, Homo sapiens
ALGPGIYSSPGSYDPR

SEQ ID NO: 320 - protein, 32 aa, Homo sapiens
NTDLDLDKKIPEDYCPLDVQIPSDLEGSAYIK

SEQ ID NO: 321 - protein, 11 aa, Homo sapiens
IGSGYDNVDIK

SEQ ID NO: 322 - protein, 16 aa, Homo sapiens
PAKLYVYINELCTVLK

SEQ ID NO: 323 - protein, 16 aa, Homo sapiens
PAKLYVYINELCTVLK

SEQ ID NO: 324 - protein, 13 aa, Homo sapiens
APLPDLYPFGTMR

SEQ ID NO: 325 - protein, 26 aa, Homo sapiens
GNQLVDLAPLGYNYTVNYTTHTQTNK

SEQ ID NO: 326 - protein, 11 aa, Homo sapiens
KQGYENLCCLR

SEQ ID NO: 327 - protein, 14 aa, Homo sapiens
TYGGCEGPDAMYVK

SEQ ID NO: 328 - protein, 20 aa, Homo sapiens
TEDQRPQLDPYQILGPTSSR

SEQ ID NO: 329 - protein, 12 aa, Homo sapiens
SKPNMNYDKLSR

SEQ ID NO: 330 - protein, 24 aa, Homo sapiens
QDHPSSMGVYGQESGGFSGPGENR

SEQ ID NO: 331 - protein, 14 aa, Homo sapiens
GDATVSYEDPPTAK

SEQ ID NO: 332 - protein, 24 aa, Homo sapiens
QQAAYYAQTSPQGMPQHPPAPQGQ

SEQ ID NO: 333 - protein, 25 aa, Homo sapiens
IDSIPHLNNSTPLVDPSVYGYGVQK

SEQ ID NO: 334 - protein, 20 aa, Homo sapiens
NTEQRPQPDPYQILGPTSSR

SEQ ID NO: 335 - protein, 12 aa, Homo sapiens
SKPNMNYDKLSR

SEQ ID NO: 336 - protein, 27 aa, Homo sapiens
TGASYYGEQTLHYIATNGESAVVQLPK

SEQ ID NO: 337 - protein, 16 aa, Homo sapiens
GIYAYGFEKPSAIQQR

SEQ ID NO: 338 - protein, 21 aa, Homo sapiens
IMGPNYTPGKKEDLYLKPIQR

SEQ ID NO: 339 - protein, 26 aa, Homo sapiens
LMEPIYLVEIQCPEQVVGGIYGVLNR

SEQ ID NO: 340 - protein, 22 aa, Homo sapiens
STAISLFYELSENDLNFIKQSK

SEQ ID NO: 341 - protein, 12 aa, Homo sapiens
VDKEKGYIDLSK

SEQ ID NO: 342 - protein, 12 aa, Homo sapiens
LYFLQCETCHSR

SEQ ID NO: 343 - protein, 12 aa, Homo sapiens
DGATILTHAYSR

SEQ ID NO: 344 - protein, 12 aa, Homo sapiens
YNIEVVCEYIVK

SEQ ID NO: 345 - protein, 34 aa, Homo sapiens
TERPVNSAALSPNYDHVVLGGGQEAMDVTTTSTR

SEQ ID NO: 346 - protein, 8 aa, Homo sapiens
YAYYDTER

SEQ ID NO: 347 - protein, 20 aa, Homo sapiens
MTLDTLSIYETPSMGLLDKK

SEQ ID NO: 348 - protein, 17 aa, Homo sapiens
TVSDLIDQKVYELQASR

SEQ ID NO: 349 - protein, 23 aa, Homo sapiens
VSSDVIDQKVYEIQDIYENSWTK

SEQ ID NO: 350 - protein, 13 aa, Homo sapiens
KQPALDVLYDVMK

SEQ ID NO: 351 - protein, 12 aa, Homo sapiens
FNVLQYVVPEVK

SEQ ID NO: 352 - protein, 12 aa, Homo sapiens
DMPYQPFSKGDR

SEQ ID NO: 353 - protein, 16 aa, Homo sapiens
GIYAYGFEKPSAIQQR

SEQ ID NO: 354 - protein, 14 aa, Homo sapiens
GYDVIAQAQSGTGK

SEQ ID NO: 355 - protein, 30 aa, Homo sapiens
LKGFGYAEFEDLDSLLSALSLNEESLGNRR

SEQ ID NO: 356 - protein, 7 aa, Homo sapiens
AFGSGYR

SEQ ID NO: 357 - protein, 21 aa, Homo sapiens
WHSNDDDVYRAPPIDRSILPT

SEQ ID NO: 358 - protein, 24 aa, Homo sapiens
RNDFQLIGIQDGYLSLLQDSGEVR

SEQ ID NO: 359 - protein, 22 aa, Homo sapiens
LNYMLLAKRLQEEKEKVDKQYK

SEQ ID NO: 360 - protein, 22 aa, Homo sapiens
LNYMLLAKRLQEEKEKVDKQYK

SEQ ID NO: 361 - protein, 13 aa, Homo sapiens
TAEIVYAATTSLR

SEQ ID NO: 362 - protein, 26 aa, Homo sapiens
IPTCFTLYQDEITEREAEPYYLLLPR

SEQ ID NO: 363 - protein, 31 aa, Homo sapiens
RPYTGNPQYTYNNWSPPVQSNETSNGYFLER

SEQ ID NO: 364 - protein, 22 aa, Homo sapiens
GQSTGKGPPQSPVFEGVYNNSR

SEQ ID NO: 365 - protein, 10 aa, Homo sapiens
NGTTYEGIFK

SEQ ID NO: 366 - protein, 23 aa, Homo sapiens
TTYDSSLSSYTVPLEKDNSEEFR

SEQ ID NO: 367 - protein, 23 aa, Homo sapiens
TTYDSSLSSYTVPLEKDNSEEFR

SEQ ID NO: 368 - protein, 9 aa, Homo sapiens
EQLLYLYAR

SEQ ID NO: 369 - protein, 27 aa, Homo sapiens
KLEDNYCEITGMNTLLSAPIHTQMQQK

SEQ ID NO: 370 - protein, 23 aa, Homo sapiens
VYVDDIYVIPQPHFSTDQPPLPK

SEQ ID NO: 371 - protein, 19 aa, Homo sapiens
ILAMTPESIYSNPSIQGCK

SEQ ID NO: 372 - protein, 14 aa, Homo sapiens
MYCNPDAMSSLTEK

SEQ ID NO: 373 - protein, 13 aa, Homo sapiens
TGIREDITYQQPR

SEQ ID NO: 374 - protein, 25 aa, Homo sapiens
SQELSGPASNGGISQTNTYDAQYER

SEQ ID NO: 375 - protein, 25 aa, Homo sapiens
SQELSGPASNGGISQTNTYDAQYER

SEQ ID NO: 376 - protein, 25 aa, Homo sapiens
QGWTVLHEAVSTGDPEMVYTVLQHR

SEQ ID NO: 377 - protein, 10 aa, Homo sapiens
NLEATYGTVR

SEQ ID NO: 378 - protein, 19 aa, Homo sapiens
IIASLADRPYEEPPQKPPR

SEQ ID NO: 379 - protein, 24 aa, Homo sapiens
LTLRPPSLAAPYAPVQSWQHQPEK

SEQ ID NO: 380 - protein, 32 aa, Homo sapiens
TQYMKYLYAYECEKKALSSPAELQAAIDGNRR

SEQ ID NO: 381 - protein, 32 aa, Homo sapiens; identical to SEQ ID NO: 380

SEQ ID NO: 382 - protein, 32 aa, Homo sapiens; identical to SEQ ID NO: 380

SEQ ID NO: 383 - protein, 32 aa, Homo sapiens; identical to SEQ ID NO: 380

SEQ ID NO: 384 - protein, 14 aa, Homo sapiens
LCYYIGATDDAATK

SEQ ID NO: 385 - protein, 22 aa, Homo sapiens
IVHSLDYYNTCEYPNEDEMPNR

SEQ ID NO: 386 - protein, 19 aa, Homo sapiens
VALENDDRSEEEKYTAVQR

SEQ ID NO: 387 - protein, 12 aa, Homo sapiens
MAEGGVTSEDYR

SEQ ID NO: 388 - protein, 10 aa, Homo sapiens
ELTELDIYHR

SEQ ID NO: 389 - protein, 19 aa, Homo sapiens
TVFVGNHPVSETEAYIAQR

SEQ ID NO: 390 - protein, 7 aa, Homo sapiens
RDYSYER

SEQ ID NO: 391 - protein, 14 aa, Homo sapiens
VDLYQQASPPDALR

SEQ ID NO: 392 - protein, 12 aa, Homo sapiens
SEGSEYEEIPKR

SEQ ID NO: 393 - protein, 24 aa, Homo sapiens
EKETNDDNYGPGPSLRPPNVACWR

SEQ ID NO: 394 - protein, 17 aa, Homo sapiens
LPSAQTPNGTDYVASGK

SEQ ID NO: 395 - protein, 13 aa, Homo sapiens
LNGQQAALASQYR

SEQ ID NO: 396 - protein, 13 aa, Homo sapiens
AMMPPYMFQQYPR

SEQ ID NO: 397 - protein, 9 aa, Homo sapiens
PAVLSGYFK

SEQ ID NO: 398 - protein, 9 aa, Homo sapiens
KFQYGNYCK

SEQ ID NO: 399 - protein, 10 aa, Homo sapiens
LYHNLQEYAK

SEQ ID NO: 400 - protein, 10 aa, Homo sapiens
LYHNLQEYAK

SEQ ID NO: 401 - protein, 9 aa, Homo sapiens
KVSLYGYLR

SEQ ID NO: 402 - protein, 10 aa, Homo sapiens
YLQTYGEWNR

SEQ ID NO: 403 - protein, 35 aa, Homo sapiens
MQEGSEVCSNPCLEENKPGIVYASLNHSVIGLNSR

SEQ ID NO: 404 - protein, 15 aa, Homo sapiens
NVKEAPTEYASICVR

SEQ ID NO: 405 - protein, 19 aa, Homo sapiens
KKQDLYMWLSNSPHGPSAK

SEQ ID NO: 406 - protein, 24 aa, Homo sapiens
LLTHYVKVQGLVISQMLRKSVETR

SEQ ID NO: 407 - protein, 8 aa, Homo sapiens
FIKVYWHR

SEQ ID NO: 408 - protein, 16 aa, Homo sapiens
LKENKISLESEYEKIK

SEQ ID NO: 409 - protein, 10 aa, Homo sapiens
RVDGMQYYCS

SEQ ID NO: 410 - protein, 10 aa, Homo sapiens
RVDGMQYYCS

SEQ ID NO: 411 - protein, 21 aa, Homo sapiens
GGWVYVLHPTPELWTLNLPHR

SEQ ID NO: 412 - protein, 15 aa, Homo sapiens
CGNCGEISDKWQYIR

SEQ ID NO: 413 - protein, 11 aa, Homo sapiens
SLEDPYSQQIR

SEQ ID NO: 414 - protein, 19 aa, Homo sapiens
HKIRNIEKKKLKLEDYKDR

SEQ ID NO: 415 - protein, 18 aa, Homo sapiens
GLPSISNGNYSQLQFQAR

SEQ ID NO: 416 - protein, 19 aa, Homo sapiens
CVGAELEYDSEHSDWHGFD

SEQ ID NO: 417 - protein, 11 aa, Homo sapiens
NQNIQKPEYSE

SEQ ID NO: 418 - protein, 12 aa, Homo sapiens
SYYPCYLKVSHR

SEQ ID NO: 419 - protein, 16 aa, Homo sapiens
TEKMVSISNYPLSAAL

SEQ ID NO: 420 - protein, 14 aa, Homo sapiens
VGKTYELLNCDKHK

SEQ ID NO: 421 - protein, 16 aa, Homo sapiens
AYADSYYYEDGGMKPR

SEQ ID NO: 422 - protein, 15 aa, Homo sapiens
MKQQALTEFEAYKHR

SEQ ID NO: 423 - protein, 11 aa, Homo sapiens
AMSDYWVVGKK

SEQ ID NO: 424 - protein, 16 aa, Homo sapiens
NLTEQNSYSNIPHEGK

SEQ ID NO: 425 - protein, 13 aa, Homo sapiens
QVAPASGYLTFER

SEQ ID NO: 426 - protein, 16 aa, Homo sapiens
VKEQKDYWETELLQLK

SEQ ID NO: 427 - protein, 12 aa, Homo sapiens
CYYPDTEEIYKL

SEQ ID NO: 428 - protein, 11 aa, Homo sapiens
IRNYYFCSLPR

SEQ ID NO: 429 - protein, 11 aa, Homo sapiens
IRNYYFCSLPR

SEQ ID NO: 430 - protein, 12 aa, Homo sapiens
EMNDAAMFYTNR

SEQ ID NO: 431 - protein, 9 aa, Homo sapiens
AYIYKCVNT

SEQ ID NO: 432 - protein, 27 aa, Homo sapiens
SRPSEAEEVPVSFDQPPEGSHLPCYNK

SEQ ID NO: 433 - protein, 15 aa, Homo sapiens
IGYFPPGIVEVVSKR

SEQ ID NO: 434 - protein, 11 aa, Homo sapiens
AQEEYEQIQAK

SEQ ID NO: 435 - protein, 20 aa, Homo sapiens
QYEQMHKELTDKLEHLEQEK

SEQ ID NO: 436 - protein, 16 aa, Homo sapiens
WREYVSQVSWGKLKRR

SEQ ID NO: 437 - protein, 32 aa, Homo sapiens
TAPVQAPPAPVIVTETPEPAMTSGVYRPPGAR

SEQ ID NO: 438 - protein, 21 aa, Homo sapiens
LQLDNQYAVLENQKSSHSQYN

SEQ ID NO: 439 - protein, 32 aa, Homo sapiens
SGGSNAEPARPDITYQEPQGTQPAQQQQPCLY

SEQ ID NO: 440 - protein, 12 aa, Homo sapiens
AARTLYRIELLR

SEQ ID NO: 441 - protein, 18 aa, Homo sapiens
RLTSECVSSNPYSKPSSK

SEQ ID NO: 442 - protein, 10 aa, Homo sapiens
SQYERPNETK

SEQ ID NO: 443 - protein, 12 aa, Homo sapiens
NSHSLYLAGIFR

SEQ ID NO: 444 - protein, 20 aa, Homo sapiens
IVNDDQSFYADIYMEDGLIK

SEQ ID NO: 445 - protein, 17 aa, Homo sapiens
THNSSLEYNIFEGMECR

SEQ ID NO: 446 - protein, 15 aa, Homo sapiens
GLYDGPVCEVSVTPK

SEQ ID NO: 447 - protein, 19 aa, Homo sapiens
FMGKTDGDYYTLDDMFVSK

SEQ ID NO: 448 - protein, 8 aa, Homo sapiens
QIKYPFAR

SEQ ID NO: 449 - protein, 21 aa, Homo sapiens
YQEYIPVQTGAHADLNPFYQK

SEQ ID NO: 450 - protein, 24 aa, Homo sapiens
MLVLDEADEMLNKGFKEQIYDVYR

SEQ ID NO: 451 - protein, 12 aa, Homo sapiens
GVTQYYAYVTER

SEQ ID NO: 452 - protein, 27 aa, Homo sapiens
NLITYDDRFNLKSIEEQLGTEIKPIPS

SEQ ID NO: 453 - protein, 15 aa, Homo sapiens
ENYLEENLLYAPSLR

SEQ ID NO: 454 - protein, 17 aa, Homo sapiens
NDGYVHIHPSSVNYQVR

SEQ ID NO: 455 - protein, 17 aa, Homo sapiens
NDGYVHIHPSSVNYQVR

SEQ ID NO: 456 - protein, 16 aa, Homo sapiens
MMNGGHYTYSENRVEK

SEQ ID NO: 457 - protein, 25 aa, Homo sapiens
TSHALDSHILDYYENPAIKEDVSTL

SEQ ID NO: 458 - protein, 25 aa, Homo sapiens
TSHALDSHILDYYENPAIKEDVSTL

SEQ ID NO: 459 - protein, 11 aa, Homo sapiens
LEEEALYAAQR

SEQ ID NO: 460 - protein, 27 aa, Homo sapiens
IVQQYHPSNNGEYQSSGPEDDFESCLR

SEQ ID NO: 461 - protein, 27 aa, Homo sapiens
IVQQYHPSNNGEYQSSGPEDDFESCLR

SEQ ID NO: 462 - protein, 28 aa, Homo sapiens
NKAKPEPDILEEEKIYAYPSNITSETGF

SEQ ID NO: 463 - protein, 27 aa, Homo sapiens
AKPEPDILEEEKIYAYPSNITSETGFR

SEQ ID NO: 464 - protein, 13 aa, Homo sapiens
AYYEFREEAYHSR

SEQ ID NO: 465 - protein, 12 aa, Homo sapiens
YYRNYYGYQGYR

SEQ ID NO: 466 - protein, 27 aa, Homo sapiens
AGCSPSDIGIIAPYRQQLKIINDLLAR

SEQ ID NO: 467 - protein, 32 aa, Homo sapiens
LAGRDTLYSCAAMPNSASRDEFPCGFTSPANR

SEQ ID NO: 468 - protein, 21 aa, Homo sapiens
GSLSTDKDTAYGSFQNGHGIK

SEQ ID NO: 469 - protein, 10 aa, Homo sapiens
KTQIYSDPLR

SEQ ID NO: 470 - protein, 19 aa, Homo sapiens
SSGPEFLQEVYTAVTYHNK

SEQ ID NO: 471 - protein, 31 aa, Homo sapiens
NHPFYVPLGAVDPGLLGYNVPALYSSDPAAR

SEQ ID NO: 472 - protein, 14 aa, Homo sapiens
CWDPSPQAYFTLPR

SEQ ID NO: 473 - protein, 14 aa, Homo sapiens
EISEASENIYSDVR

SEQ ID NO: 474 - protein, 12 aa, Homo sapiens
IYSYVVSRPQPR

SEQ ID NO: 475 - protein, 14 aa, Homo sapiens
SRDHVVEGEPYAGY

SEQ ID NO: 476 - protein, 12 aa, Homo sapiens
SFDPASEEVYAK

SEQ ID NO: 477 - protein, 13 aa, Homo sapiens
FSAHYDAVEAELK

SEQ ID NO: 478 - protein, 11 aa, Homo sapiens
SSATVYFQTVK

SEQ ID NO: 479 - protein, 20 aa, Homo sapiens
PHDGPPAAVYSNLGAYRPTR

SEQ ID NO: 480 - protein, 10 aa, Homo sapiens
TITGNIYYAR

SEQ ID NO: 481 - protein, 25 aa, Homo sapiens
NSQLDKNSEVYQEVQAMFDTLGIPK

SEQ ID NO: 482 - protein, 20 aa, Homo sapiens
RVEAMKQYQEEIQELNEVAR

SEQ ID NO: 483 - protein, 20 aa, Homo sapiens
KPGADLSDYFNYGFNEDTWK

SEQ ID NO: 484 - protein, 34 aa, Homo sapiens
AFPYGNVAFPHLPGSAPSWPSLVDTSKQWDYYAR

SEQ ID NO: 485 - protein, 15 aa, Homo sapiens
QAEEYLSFAFEHCHR

SEQ ID NO: 486 - protein, 6 aa, Homo sapiens
HEYLPK

SEQ ID NO: 487 - protein, 10 aa, Homo sapiens
DSDYYNMLLK

SEQ ID NO: 488 - protein, 11 aa, Homo sapiens
LQNNNVYTIAK

SEQ ID NO: 489 - protein, 20 aa, Homo sapiens
CRAPEVSQYIYQVYDSILKN

SEQ ID NO: 490 - protein, 37 aa, Homo sapiens
AGLGEGVPPGNYGNYGYANSGYSACEEENERLTESLR

SEQ ID NO: 491 - protein, 7 aa, Homo sapiens
AYEFAER

SEQ ID NO: 492 - protein, 27 aa, Homo sapiens
LAELEEFINGPNNAHIQQVGDRCYDEK

SEQ ID NO: 493 - protein, 11 aa, Homo sapiens
CYDEKMYDAAK

SEQ ID NO: 494 - protein, 12 aa, Homo sapiens
VHMFEAHSDYIR

SEQ ID NO: 495 - protein, 14 aa, Homo sapiens
LQDAYYIFQEMADK

SEQ ID NO: 496 - protein, 19 aa, Homo sapiens
VYSPHVLNLTLIDLPGITK

SEQ ID NO: 497 - protein, 19 aa, Homo sapiens
EPELFQTVAEGLRQLYAQK

SEQ ID NO: 498 - protein, 18 aa, Homo sapiens
DKPTYDEIFYTLSPVNGK

SEQ ID NO: 499 - protein, 12 aa, Homo sapiens
NIVHNYSEAEIK

(19) United States
(12) Patent Application Publication
(10) Pub. No.: US 2010/0151483 A1
(43) Pub. Date: Jun. 17, 2010

(54) REAGENTS FOR THE DETECTION OF PROTEIN PHOSPHORYLATION IN SIGNALING PATHWAYS

(75) Inventors: Peter Hornbeck, Magnolia, MA (US); Valerie Goss, Seabrook, NH (US); Kimberly Lee, Seattle, WA (US); Ting-Lei Gu, Woburn, MA (US); Albrecht Moritz, Salem, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC., Danvers, MA (US)

(21) Appl. No.: 12/309,312

(22) PCT Filed: Jul. 13, 2007
(86) PCT No.: PCT/US2007/073534

§ 371 Date: Feb. 19, 2010

Related U.S. Application Data
(60) Provisional application No. 60/830,549, filed on Jul. 13, 2006.

(51) Int. Cl. G01N 33/53; C07K 16/18

(52) U.S. Cl. 435/7.1; 530/387.9

(57) ABSTRACT

The invention discloses novel phosphorylation sites identified in signal transduction proteins and pathways, and provides phosphorylation-site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: adaptor/scaffold proteins, adhesion/extracellular matrix protein, apoptosis proteins, calcium binding proteins, cell cycle regulation proteins, chaperone proteins, chromatin, DNA binding/repair/replication proteins, cytoskeletal proteins, endoplasmic reticulum or golgi proteins, enzyme proteins, G/regulator proteins, inhibitor proteins, motor/contractile proteins, phosphatase, protease, Ser/Thr protein kinases, protein kinase (Tyr)s, receptor/channel/cell surface proteins, RNA binding proteins, transcriptional regulators, tumor suppressor proteins, ubiquitan conjugating system proteins and proteins of unknown function.

### RELATED APPLICATIONS

Pursuant to 35 U.S.C. §119(e) this application claims the benefit of, and priority to, provisional application U.S. Ser. No. 60/830,549, filed Jul. 13, 2006, the disclosure of which is incorporated herein, in its entirety, by reference

### TECHNICAL FIELD

The invention relates generally to a variety of moieties and tools for the detection of protein phosphorylation. Moreover, the invention relates to the use of the same for diagnostic and therapeutic purposes.

### BACKGROUND

The activation of proteins by post-translational modification is an important cellular mechanism for regulating most aspects of biological organization and control, including growth, development, homeostasis, and cellular communication. Cellular signal transduction pathways involve protein kinases, protein phosphatases, and phosphoprotein-interacting domain (e.g., SH2, PTB, WW, FHA, 14-3-3) containing cellular proteins to provide multidimensional, dynamic and reversible regulation of many biological activities. See e.g., Sawyer et al., Med. Chem. 1(3): 293-319 (2005).

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82: 111-21 (1999).

Many of these phosphorylation sites regulate critical biological processes and may prove to be important for diagnostic or therapeutic modalities useful in the treatment and management of many pathological conditions and diseases, including inter alia cancer, developmental disorders, as inflammatory, immune, metabolic and bone diseases.

For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of many disease states.

Understanding reversible protein phosphorylation and its role in the operation and interrelationship between cellular components and functions provides the opportunity to gain a finer appreciation of cellular regulation. In spite of the importance of protein modification, phosphorylation is not yet well understood due to the extraordinary complexity of signaling pathways, and the slow development of the technology necessary to unravel it.

In many instances, such knowledge is likely to provide valuable tools useful to evaluate, and possibly to manipulate target pathways, ultimately altering the functional status of a given cell for a variety of purposes.

The importance of protein kinase-regulated signal transduction pathways is underscored by a number of drugs designed to treat various cancer types by the inhibition of target protein kinases at the apex or intermediary levels of pathways implicated in cancer development. See Stern et al., Expert Opin. Ther. Targets 9(4):851-60 (2005).

Leukemia, a disease in which a number of underlying signal transduction events have been elucidated, has become a disease model for phosphoproteomic research and development efforts. As such, it represent a paradigm leading the way for many other programs seeking to address many classes of diseases (See, Harrison's Principles of Internal Medicine, McGraw-Hill, New York, N.Y.)

Depending on the cell type involved and the rate by which the disease progresses leukemia can be defined as acute or chronic myelogenous leukemia (AML or CML), or acute and chronic lymphocytic leukemia (ALL or CLL).

Most varieties of leukemia are generally characterized by genetic alterations e.g., chromosomal translocations, deletions or point mutations resulting in the constitutive activation of protein kinase genes, and their products, particularly tyrosine kinases. The most well known alteration is the oncogenic role of the chimeric BCR-Abl gene. See Nowell, Science 132: 1497 (1960)). The resulting BCR-Abl kinase protein is constitutively active and elicits characteristic signaling pathways that have been shown to drive the proliferation and survival of CML cells (see Daley, Science 247: 824-830 (1990); Raitano et al., Biochim. Biophys. Acta. December 9; 1333(3): F201-16 (1997)).

The recent success of Imanitib (also known as STI571 or Gleevec®), the first molecularly targeted compound designed to specifically inhibit the tyrosine kinase activity of BCR-Abl, provided critical confirmation of the central role of BCR-Abl signaling in the progression of CML (see Schindler et al., Science 289: 1938-1942 (2000); Nardi et al., Curr. Opin. Hematol. 11:35-43 (2003)).

The success of Gleevec® now serves as a paradigm for the development of targeted drugs designed to block the activity of other tyrosine kinases known to be involved in many diseased including leukemias and other malignancies (see, e.g., Sawyers, Curr. Opin. Genet. Dev. February; 12(1):111-5 (2002); Druker, Adv. Cancer Res. 91:1-30 (2004)). For example, recent studies have demonstrated that mutations in the FLT3 gene occur in one third of adult patients with AML. FLT3 (Fms-like tyrosine kinase 3) is a member of the class III receptor tyrosine kinase (RTK) family including FMS, platelet-derived growth factor receptor (PDGFR) and c-KIT (see Rosnet et al., Crit. Rev. Oncog. 4: 595-613 (1993). In 20-27% of patients with AML, an internal tandem duplication in the juxta-membrane region of FLT3 can be detected (see Yokota et al., Leukemia 11: 1605-1609 (1997)). Another 7% of patients have mutations within the active loop of the second kinase domain, predominantly substitutions of aspartate residue 835 (D835), while additional mutations have been described (see Yamamoto et al., Blood 97: 2434-2439 (2001); Abu-Duhier et al., Br. J. Haematol. 113: 983-988 (2001)). Expression of mutated FLT3 receptors results in constitutive tyrosine phosphorylation of FLT3, and subsequent phosphorylation and activation of downstream molecules such as STAT5, Akt and MAPK, resulting in factor-independent growth of hematopoietic cell lines.

Altogether, FLT3 is the single most common activated gene in AML known to date. This evidence has triggered an intensive search for FLT3 inhibitors for clinical use leading to at least four compounds in advanced stages of clinical development, including: PKC412 (by Novartis), CEP-701 (by Cephalon), MLN518 (by Millenium Pharmaceuticals), and SU5614 (by Sugen/Pfizer) (see Stone et al., Blood (in press) (2004); Smith et al., Blood 103: 3669-3676 (2004); Clark et al., Blood 104: 2867-2872 (2004); and Spiekerman et al., Blood 101: 1494-1504 (2003)).

There is also evidence indicating that kinases such as FLT3, c-KIT and Abl are implicated in some cases of ALL (see Cools et al., Cancer Res. 64: 6385-6389 (2004); Hu, Nat. Genet. 36: 453-461 (2004); and Graux et al., Nat. Genet. 36: 1084-1089 (2004)). In contrast, very little is know regarding any causative role of protein kinases in CLL, except for a high correlation between high expression of the tyrosine kinase ZAP70 and the more aggressive form of the disease (see Rassenti et al., N. Eng. J. Med. 351: 893-901 (2004)).

Despite the identification of a few key molecules involved in progression of leukemia, the vast majority of signaling protein changes underlying this disease remains unknown. There is, therefore, relatively scarce information about kinase-driven signaling pathways and phosphorylation sites relevant to the different types of leukemia. This has hampered a complete and accurate understanding of how protein activation within signaling pathways is driving these complex cancers. Accordingly, there is a continuing and pressing need to unravel the molecular mechanisms of kinase-driven oncogenesis in leukemia by identifying the downstream signaling proteins mediating cellular transformation in this disease. Identifying particular phosphorylation sites on such signaling proteins and providing new reagents, such as phospho-specific antibodies and AQUA peptides, to detect and quantify them remains particularly important to advancing our understanding of the biology of this disease.

Presently, diagnosis of leukemia is made by tissue biopsy and detection of different cell surface markers. However, misdiagnosis can occur since some leukemia cases can be negative for certain markers, and because these markers may not indicate which genes or protein kinases may be deregulated. Although the genetic translocations and/or mutations characteristic of a particular form of leukemia can be sometimes detected, it is clear that other downstream effectors of constitutively active kinases having potential diagnostic, predictive, or therapeutic value, remain to be elucidated. Accordingly, identification of downstream signaling molecules and phosphorylation sites involved in different types of leukemia and development of new reagents to detect and quantify these sites and proteins may lead to improved diagnostic/prognostic markers, as well as novel drug targets, for the detection and treatment of this disease.

### SUMMARY OF THE INVENTION

Several novel protein phosphorylation sites have been identified in a variety of cell lines. Such novel phosphorylation sites (tyrosine), and their corresponding parent proteins are reported (see Table 1). The elucidation of these sites at long last provides the elements necessary to attain those much needed proteomics tools and modalities.

The invention discloses novel phosphorylation sites identified in signal transduction proteins and pathways underlying various disease states including for example human leukemias. The invention thus provides new reagents, including phosphorylation-site specific antibodies and AQUA peptides, for the selective detection and quantification of these phosphorylated sites/proteins. Also provided are methods of using the reagents of the invention for the detection and quantification of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the Leukemia signaling protein phosphorylation sites disclosed herein:

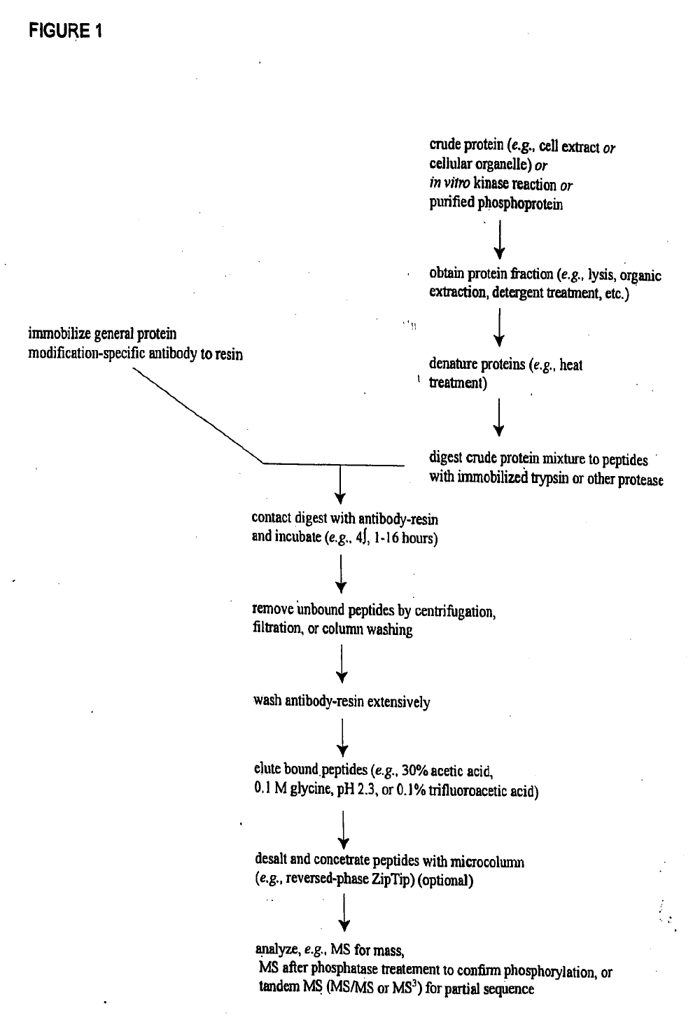

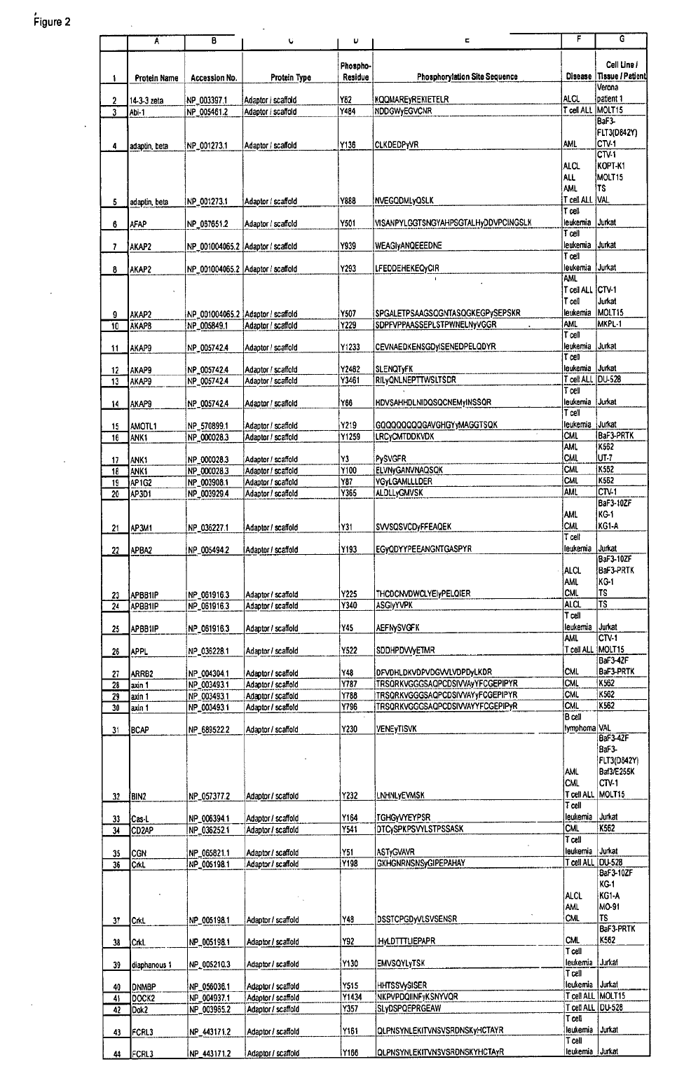

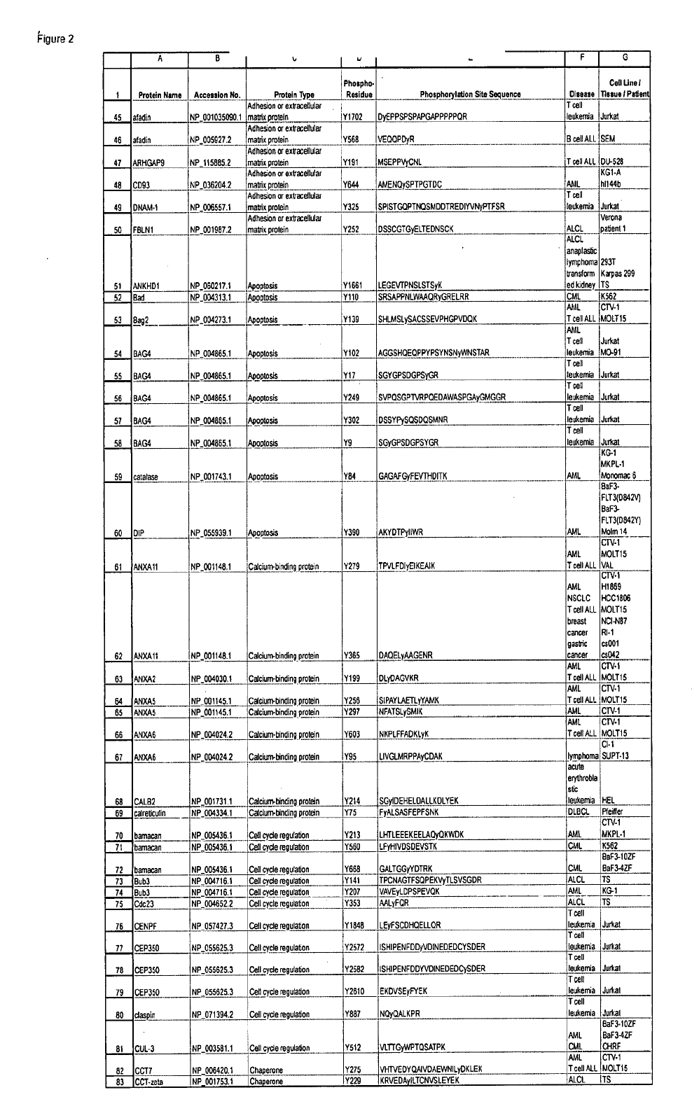

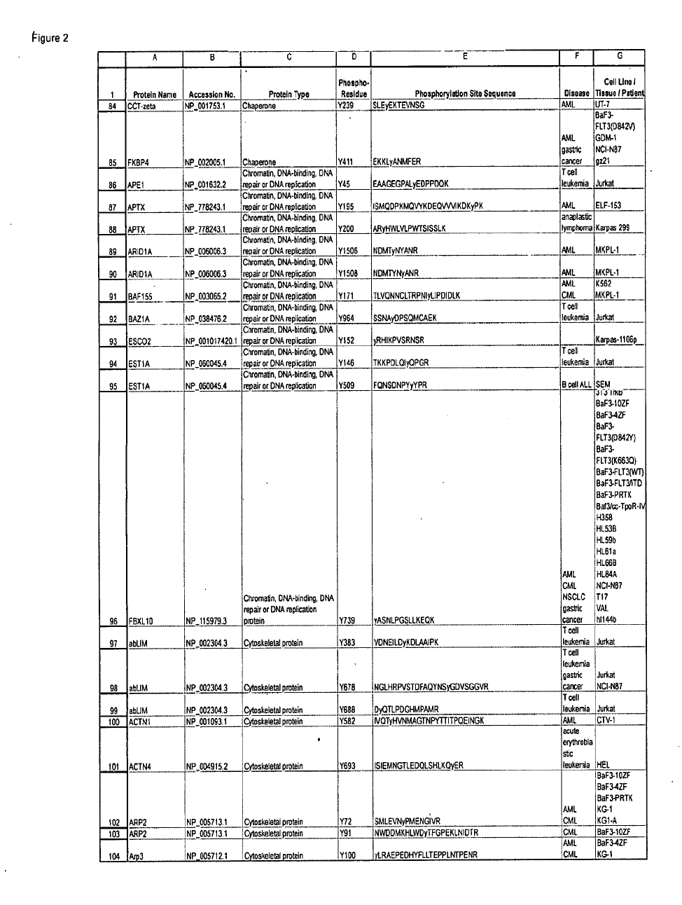

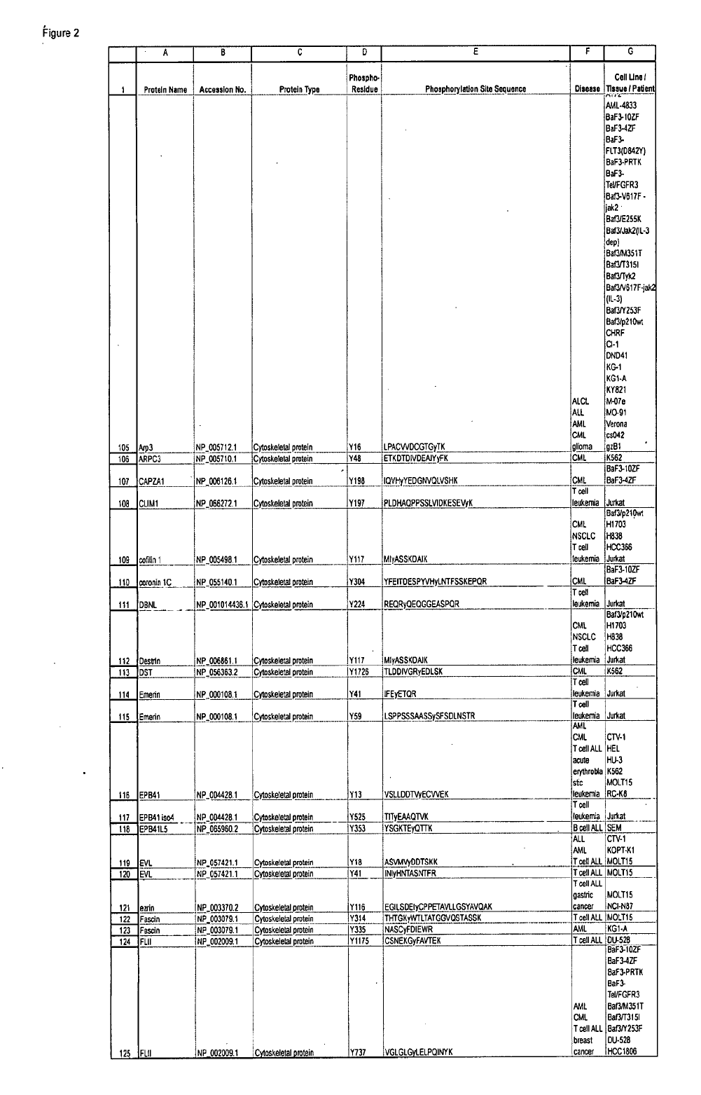

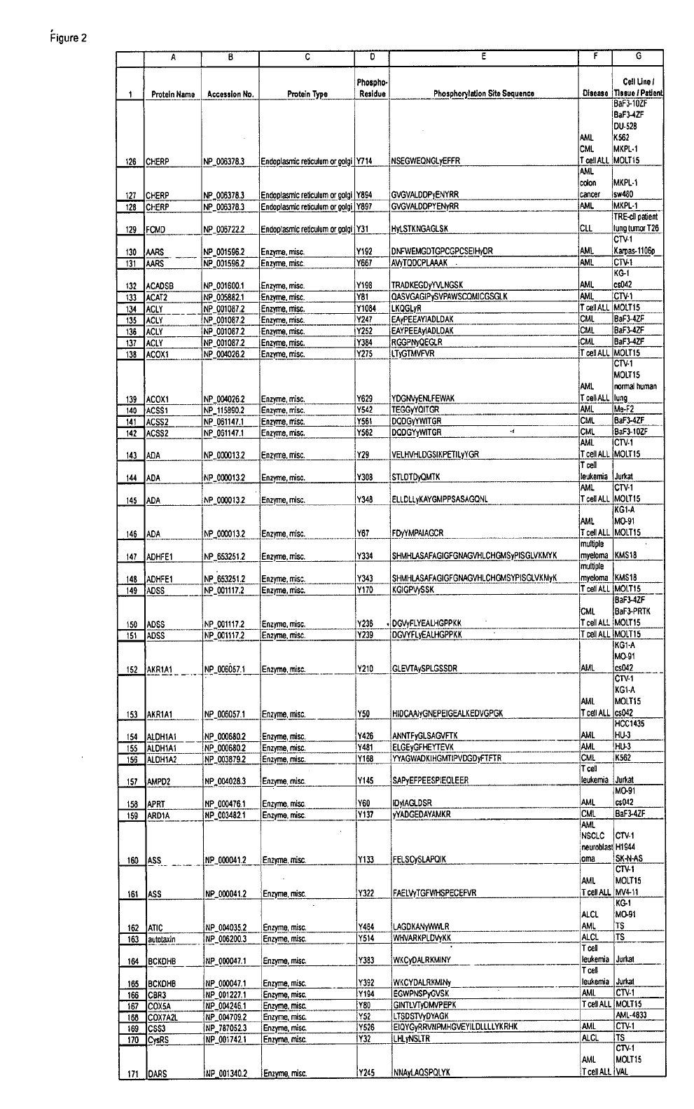

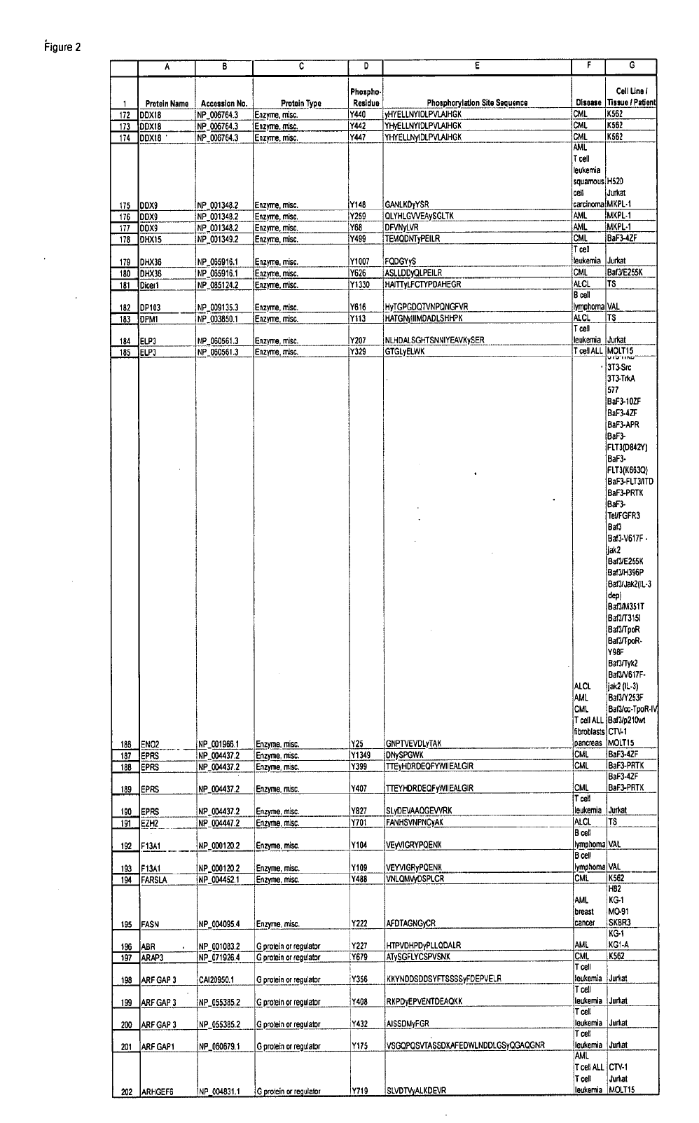

Column A=the name of the parent protein; Column B=the SwissProt accession number for the protein (human sequence); Column C=the protein type/classification; Column D=the tyrosine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the type of leukemia in which the phosphorylation site was discovered; and Column G=the cell type(s), tissue(s) and/or patient(s) in which the phosphorylation site was discovered.

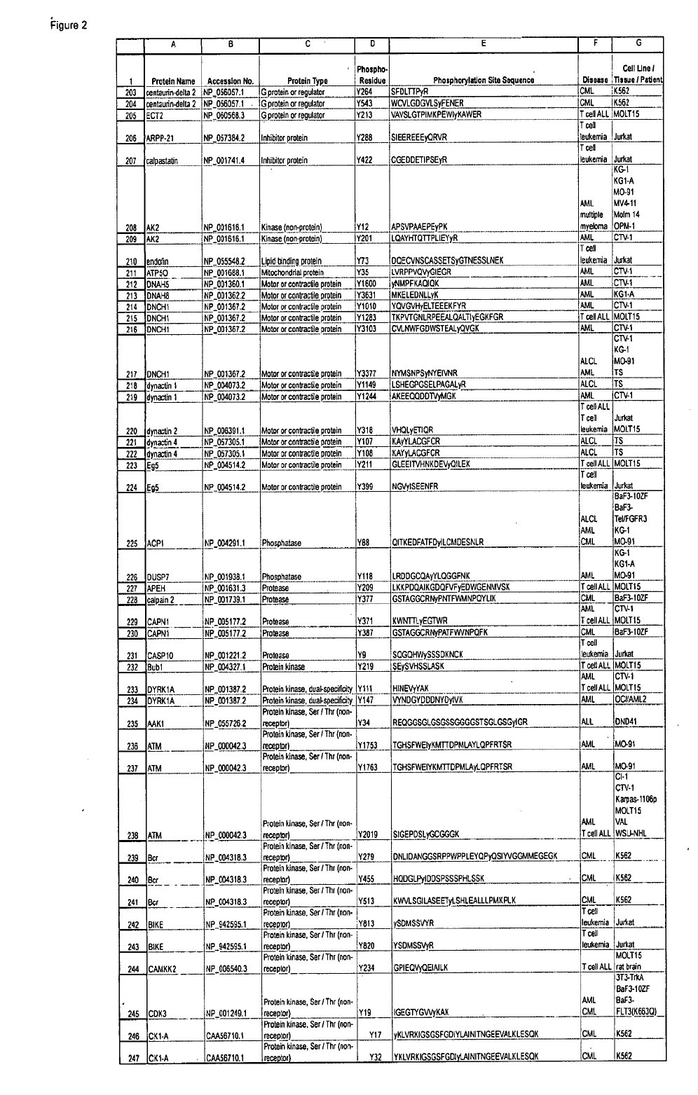

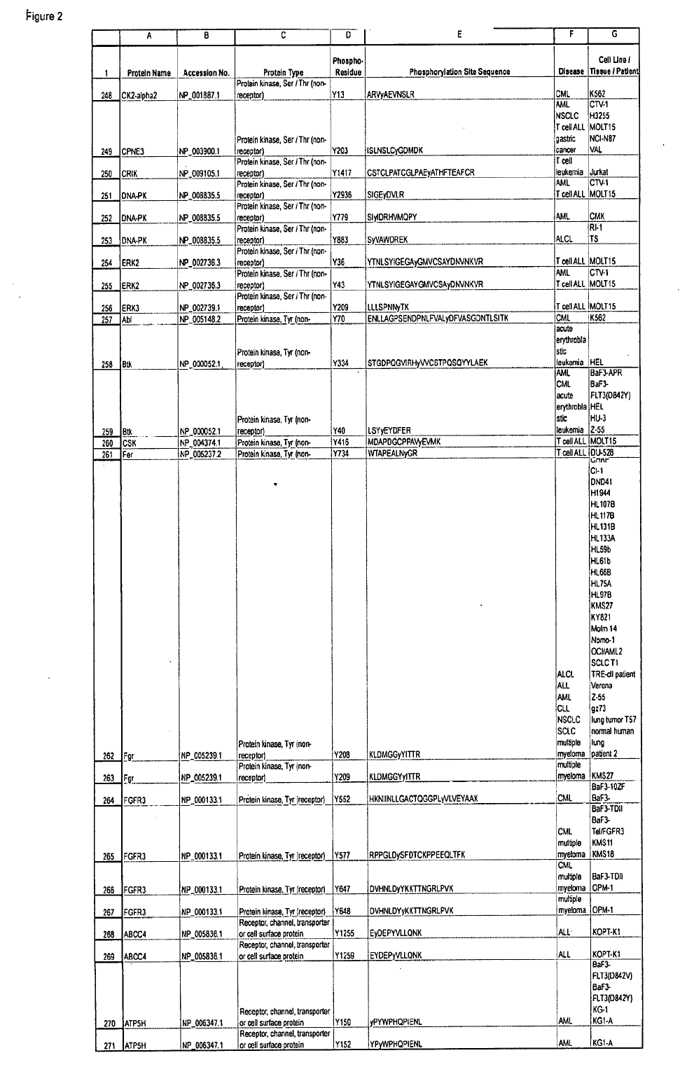

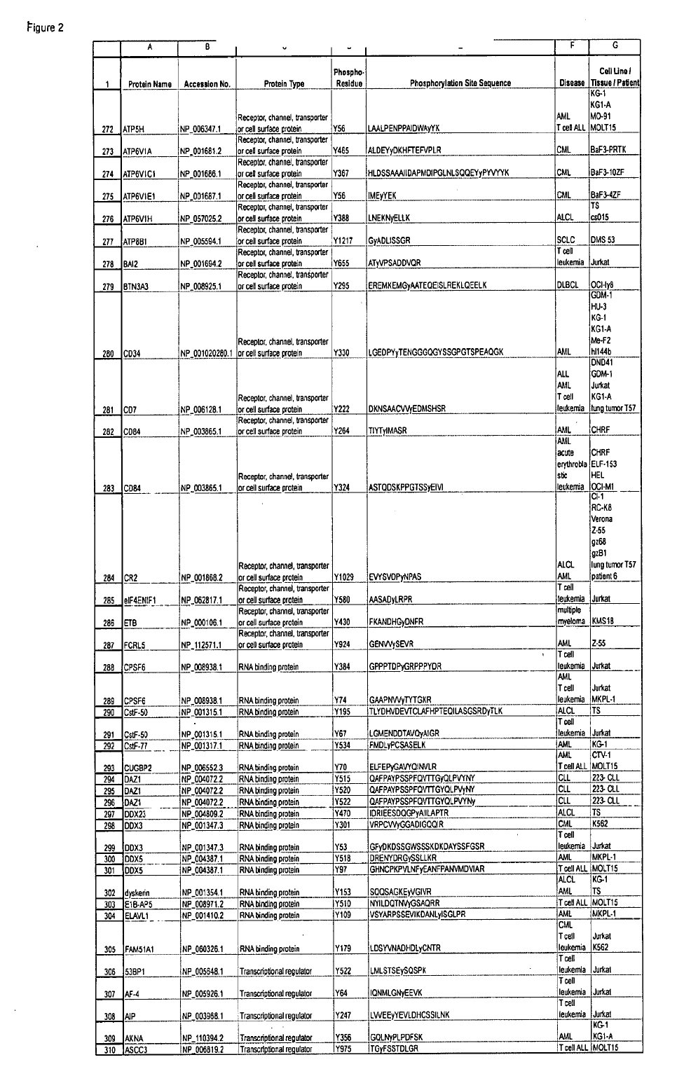

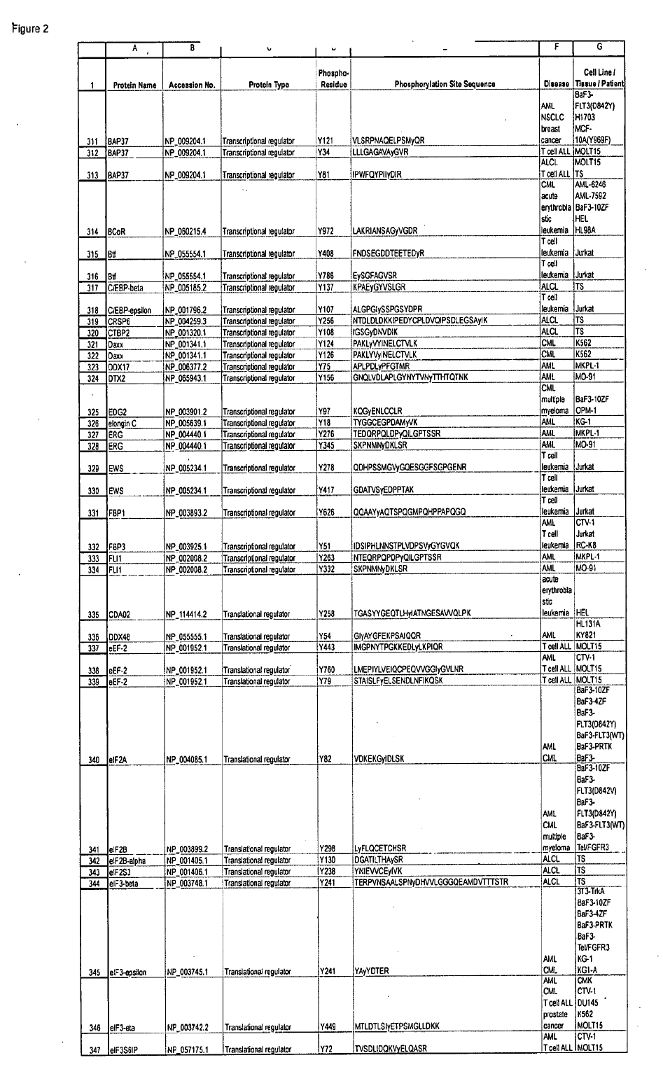

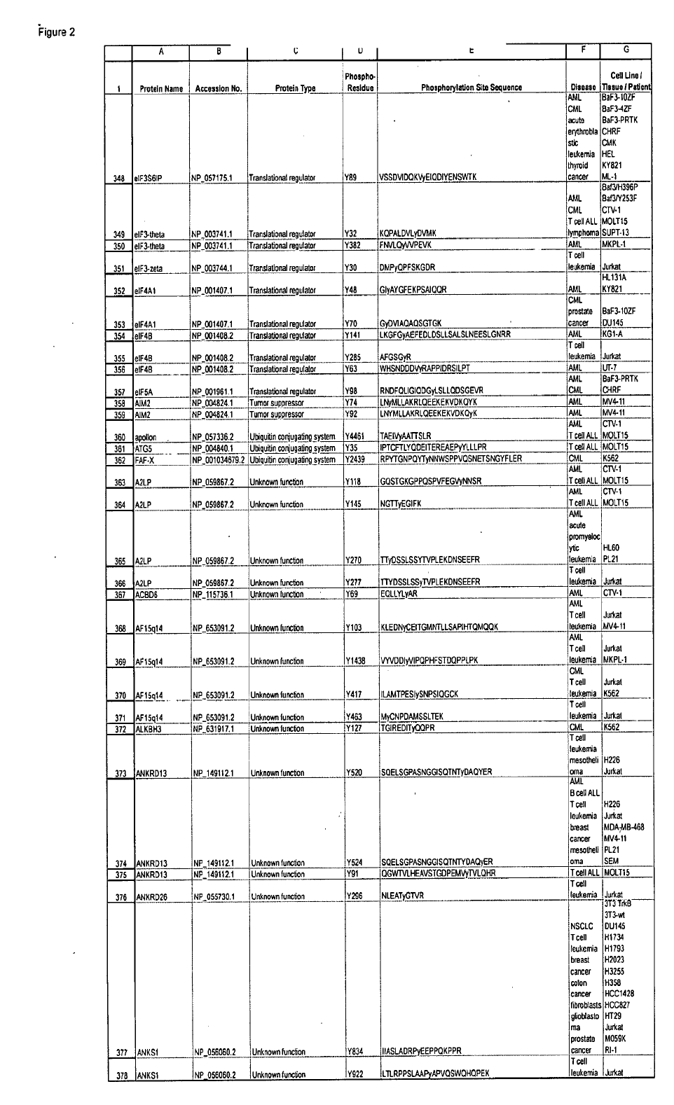

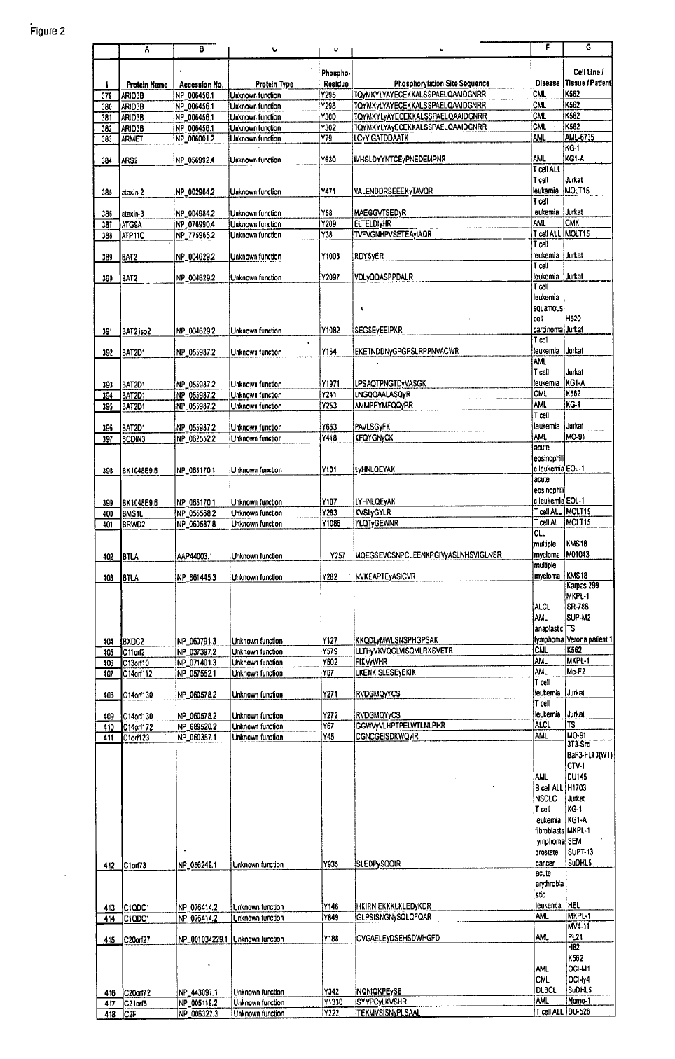

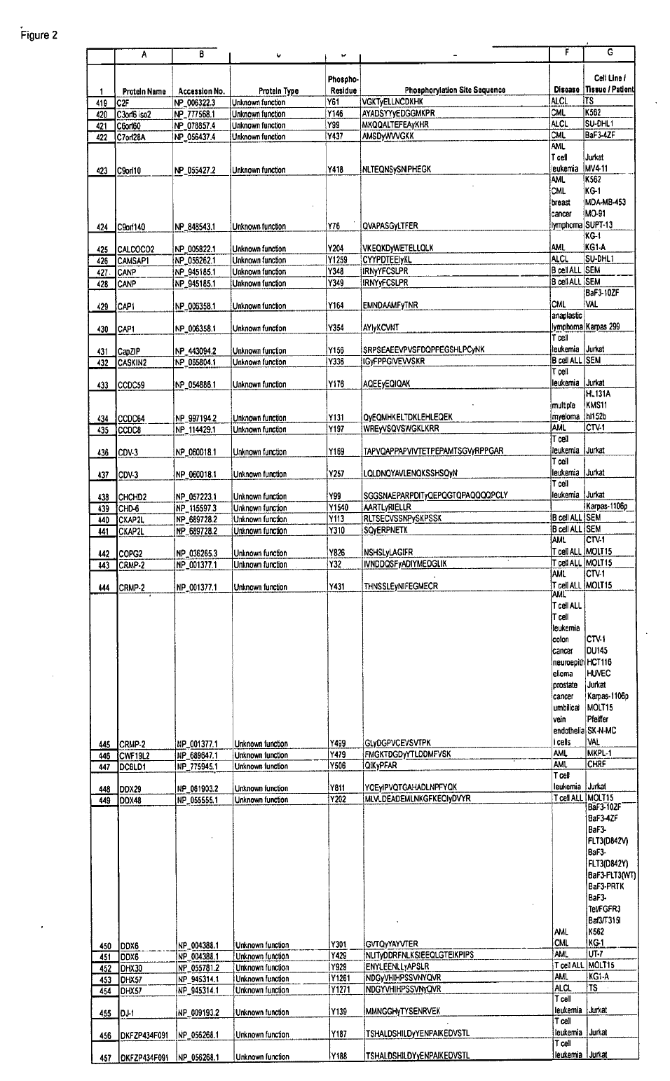

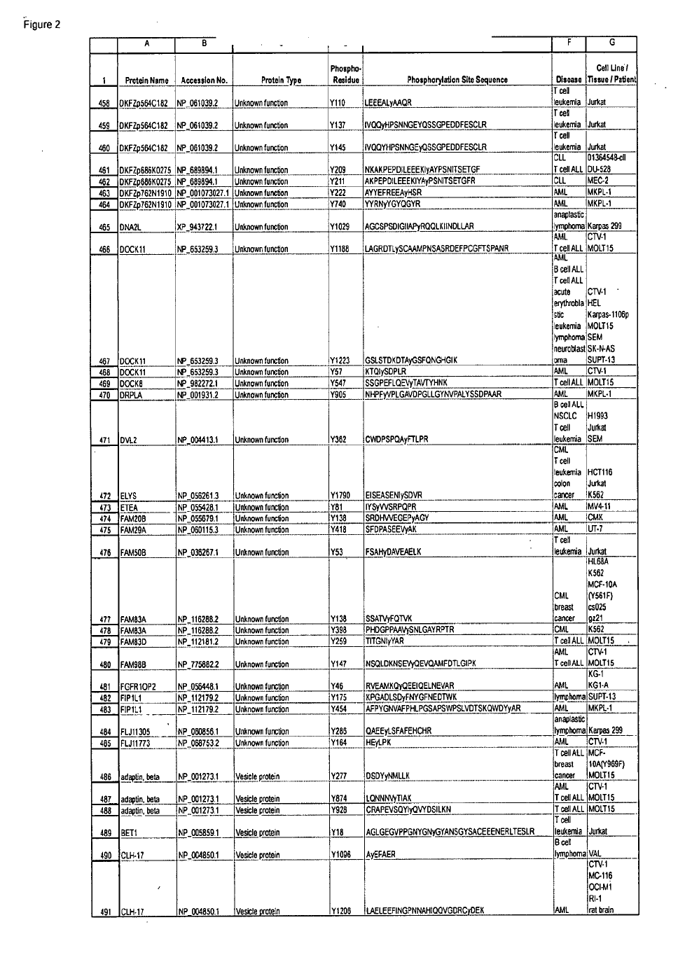

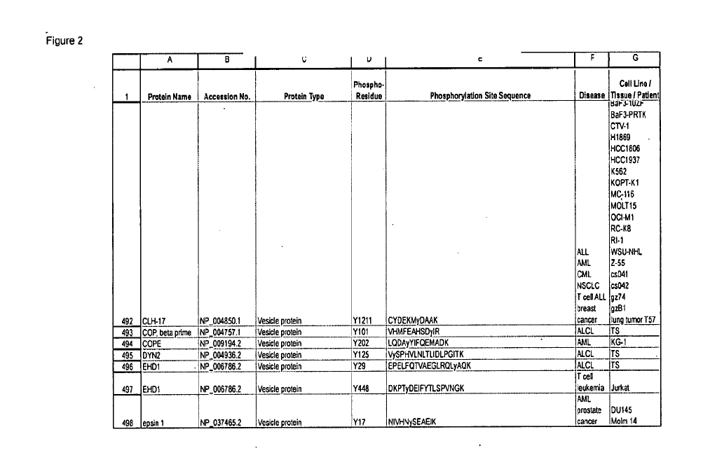

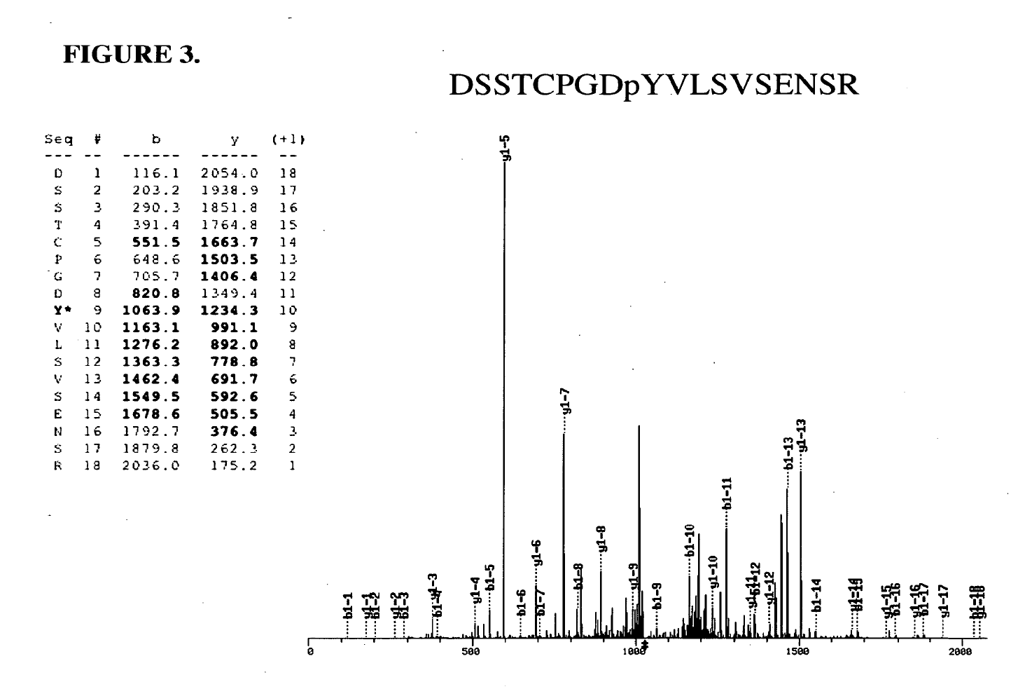

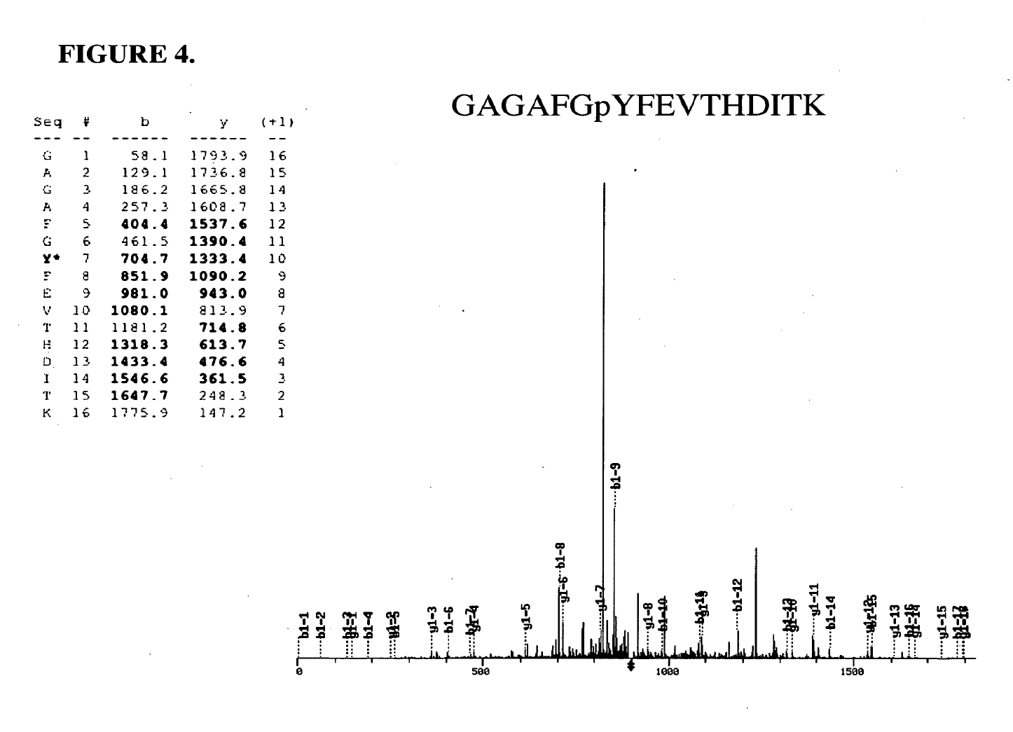

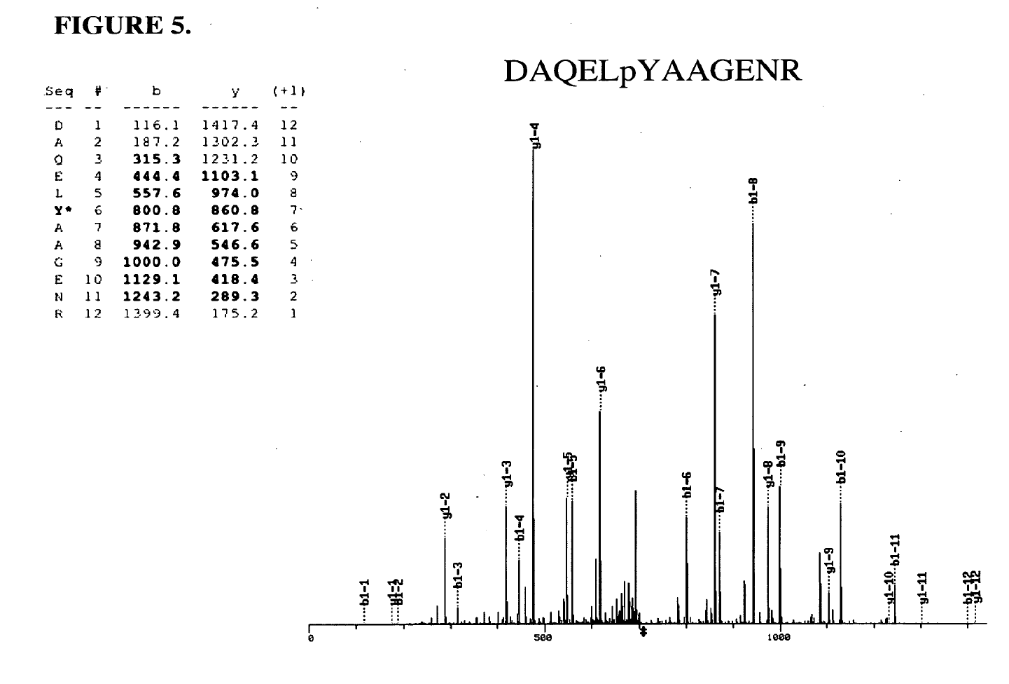

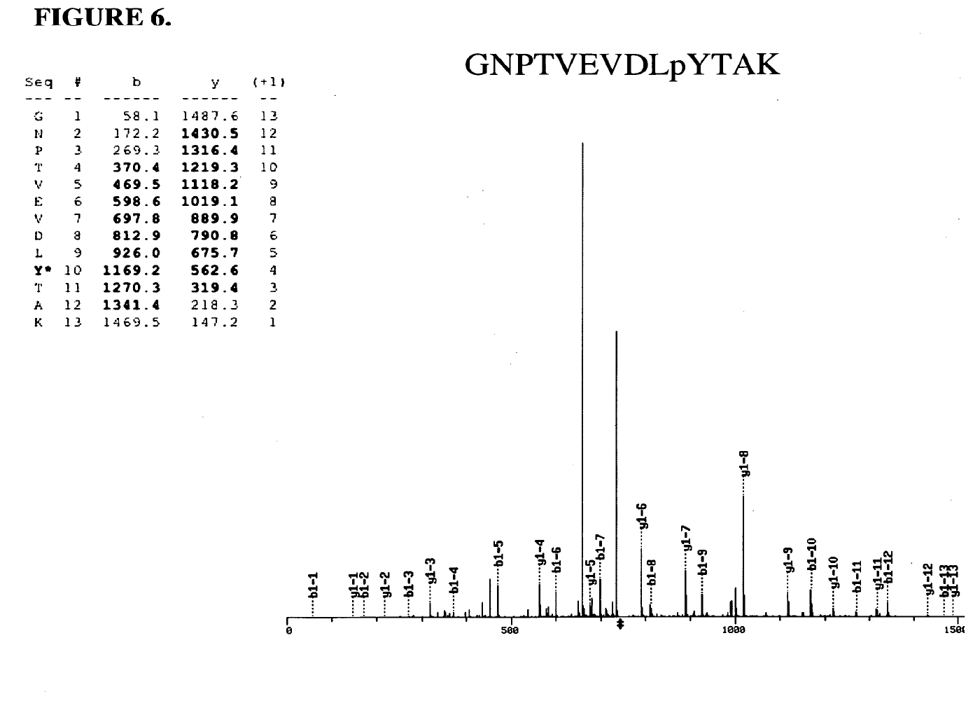

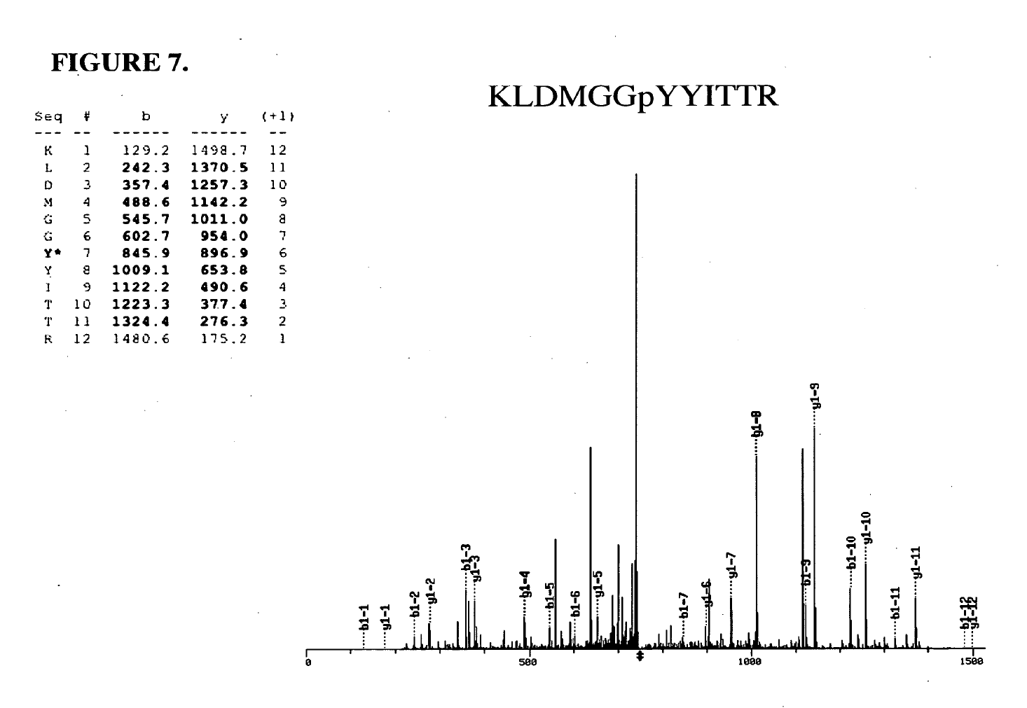

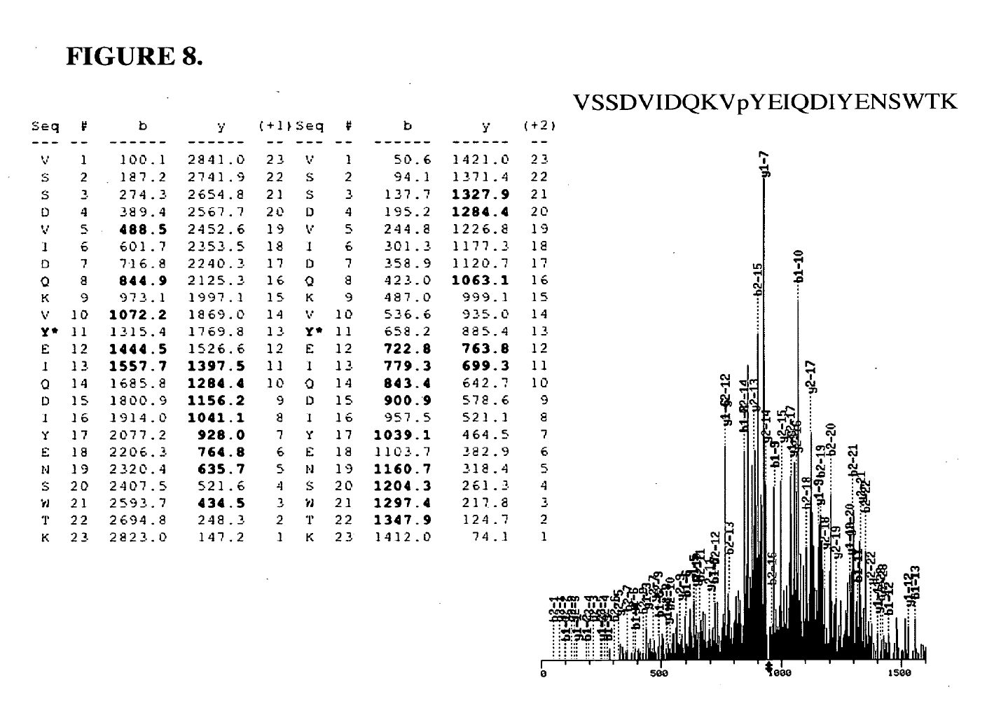

FIG. 3—is an exemplary mass spectrograph depicting the detection of the tyrosine 48 phosphorylation site in CRKL (see Row 37 in FIG. 2/Table 1), as further described in Example 1 (red and blue indicate ions detected in MS/MS spectrum); Y* indicates the phosphorylated tyrosine (shown as lowercase “y” in FIG. 2).

FIG. 4—is an exemplary mass spectrograph depicting the detection of the tyrosine 83 phosphorylation site in Catalase (see Row 59 in FIG. 2/Table 1), as further described in Example 1 (red and blue indicate ions detected in MS/MS spectrum); Y* indicates the phosphorylated tyrosine (shown as lowercase “y” in FIG. 2).

FIG. 5—is an exemplary mass spectrograph depicting the detection of the tyrosine 365 phosphorylation site in ANXA11 (see Row 62 in FIG. 2/Table 1), as further described in Example 1 (red and blue indicate ions detected in MS/MS spectrum); Y* indicates the phosphorylated serine (shown as lowercase “y” in FIG. 2).

FIG. 6—is an exemplary mass spectrograph depicting the detection of the tyrosine 24 phosphorylation site in ENO2 (see Row 186 in FIG. 2/Table 1), as further described in Example 1 (red and blue indicate ions detected in MS/MS spectrum); Y* indicates the phosphorylated tyrosine (shown as lowercase “y” in FIG. 2)

FIG. 7—is an exemplary mass spectrograph depicting the detection of the tyrosine 208 phosphorylation site in Fgr (see Row 262 in FIG. 2/Table 1), as further described in Example 1 (red and blue indicate ions detected in MS/MS spectrum); Y* indicates the phosphorylated tyrosine (shown as lowercase “y” in FIG. 2).

FIG. 8—is an exemplary mass spectrograph depicting the detection of the tyrosine 89 phosphorylation site in eIF3S6IP (see Row 348 in FIG. 2/Table 1), as further described in Example 1 (red and blue indicate ions detected in MS/MS spectrum); Y* indicates the phosphorylated tyrosine (shown as lowercase “y” in FIG. 2).

### DETAILED DESCRIPTION

Several novel protein phosphorylation sites have been identified in a variety of cell lines. Such novel phosphorylation sites (tyrosine), and their corresponding parent proteins are reported (see Table 1). The elucidation of these sites at long last provides the elements necessary to attain those much needed proteomics tools and modalities.

The disclosure of the phosphorylation sites provides the key to the production of new moieties, compositions and methods to specifically detect and/or to quantify these phosphorylated sites/proteins. Such moieties include for example reagents, such as phosphorylation site-specific antibodies and AQUA peptides (heavy-isotope labeled peptides). Such reagents are highly useful, inter alia, for studying signal transduction events underlying the progression of many diseases known or suspected to involve protein phosphorylation e.g., leukemia in a mammal. Accordingly, the invention provides novel reagents—phospho-specific antibodies and AQUA peptides—for the specific detection and/or quantification of a target signaling protein/polypeptide (e.g., a signaling protein/polypeptide implicated in leukemia) only when phosphorylated (or only when not phosphorylated) at a particular phosphorylation site disclosed herein. The invention also provides methods of detecting and/or quantifying one or more phosphorylated target signaling protein/polypeptide using the phosphorylation-site specific antibodies and AQUA peptides of the invention.

These phosphorylation sites correspond to numerous different parent proteins (the full sequences (human) of which are all publicly available in SwissProt database and their Accession numbers listed in Column B of Table 1/FIG. 2), each of which are have been linked to specific functions in the literature and thus may be organized into discrete protein type groups, for example adaptor/scaffold proteins, cytoskeletal proteins, protein kinases, and DNA binding proteins, etc. (see Column C of Table 1), the phosphorylation of which is relevant to signal transduction activity (e.g, underlying AML, CML, CLL, and ALL), as disclosed herein.

In part, the invention provides an isolated phosphorylation site-specific antibody that specifically binds a given target signaling protein/polypeptide only when phosphorylated (or not phosphorylated, respectively) at a particular tyrosine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given target signaling protein/polypeptide, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the AFAP adaptor/scaffold protein only when phosphorylated (or only when not phosphorylated) at tyrosine 501 (see Row 6 (and Columns D and E) of Table 1/FIG. 2). By way of further example, among the group of reagents provided by the invention is an AQUA peptide for the quantification of phosphorylated Bad apoptosis protein, the AQUA peptide comprising the phosphorylatable peptide sequence listed in Column E, Row 52, of Table 1/FIG. 2 (which encompasses the phosphorylatable tyrosine at position 110).

In one embodiment, the invention provides an isolated phosphorylation site-specific antibody that specifically binds a target signaling protein/polypeptide selected from Column A of Table 1 (Rows 2-498) only when phosphorylated at the tyrosine residue listed in corresponding Column D of Table 1, comprised within the phosphorylatable peptide sequence listed in corresponding Column E of Table 1 (SEQ ID NOs: 1-278, 280-289, 291-499), wherein said antibody does not bind said signaling protein when not phosphorylated at said tyrosine. In another embodiment, the invention provides an isolated phosphorylation site-specific antibody that specifically binds a target signaling protein/polypeptide selected from Column A of Table 1 only when not phosphorylated at the tyrosine residue listed in corresponding Column D of Table 1, comprised within the peptide sequence listed in corresponding Column E of Table 1 (SEQ ID NOs: 1-278, 280-289, 291-499), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one embodiment, the immortalized cell line is a rabbit or mouse hybridoma.

In another embodiment, the invention provides a heavy-isotope labeled peptide (AQUA peptide) for the quantification of a target signaling protein/polypeptide selected from Column A of Table 1, said labeled peptide comprising the phosphorylatable peptide sequence listed in corresponding Column E of Table 1 (SEQ ID NOs: 1-278, 280-289, 291-499), which sequence comprises the phosphorylatable tyrosine listed in corresponding Column D of Table 1. In certain embodiments, the phosphorylatable tyrosine within the labeled peptide is phosphorylated, while in other embodiments, the phosphorylatable residue within the labeled peptide is not phosphorylated.

Reagents (antibodies and AQUA peptides) provided by the invention may conveniently be grouped by the type of target signaling protein/polypeptide in which a given phosphorylation site (for which reagents are provided) occurs. The protein types for each respective protein (in which a phosphorylation site has been discovered) are provided in Column C of Table 1/FIG. 2, and include: adaptor/scaffold proteins, adhesion/extracellular matrix protein, apoptosis proteins, calcium binding proteins, cell cycle regulation proteins, chaperone proteins, chromatin, DNA binding/repair/replication proteins, cytoskeletal proteins, endoplasmic reticulum or golgi proteins, enzyme proteins, G/regulator proteins, inhibitor proteins, motor/contractile proteins, phosphatase, protease, Ser/Thr protein kinases, protein kinase (Tyr)s, receptor/channel/cell surface proteins, RNA binding proteins, transcriptional regulators, tumor suppressor proteins, ubiquitan conjugating system proteins and proteins of unknown function. Each of these distinct protein groups is a subset of target signaling protein/polypeptide phosphorylation sites disclosed herein, and reagents for their detection/quantification may be considered a subset of reagents provided by the invention.

Subsets of the phosphorylation sites (and their corresponding proteins) disclosed herein are those occurring on the following protein types/groups listed in Column C of Table 1/FIG. 2 adaptor/scaffold proteins, calcium binding proteins, chromatin or DNA binding/repair/replication proteins, cytoskeletal proteins, enzyme proteins, protein kinases (Tyr), protein kinases (Ser/Thr), receptor/channel/transporter/cell surface proteins, transcriptional regulators and translational regulators. Accordingly, among subsets of reagents provided by the invention are isolated antibodies and AQUA peptides useful for the detection and/or quantification of the foregoing protein/phosphorylation site subsets.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

In one subset of embodiments, there is provided:

(i) An isolated phosphorylation site-specific antibody that specifically binds an adaptor/scaffold protein selected from Column A, Rows 2-44, of Table 1 only when phosphorylated at the tyrosine listed in corresponding Column D, Rows 2-44, of Table 1, comprised within the phosphorylatable peptide sequence listed in corresponding Column E, Rows 2-44, of Table 1 (SEQ ID NOs: 1-43), wherein said antibody does not bind said protein when not phosphorylated at said tyrosine.
(ii) An equivalent antibody to (i) above that only binds the adaptor/scaffold protein when not phosphorylated at the disclosed site (and does not bind the protein when it is phosphorylated at the site).
(iii) A heavy-isotope labeled peptide (AQUA peptide) for the quantification of an adaptor/scaffold protein selected from Column A, Rows 2-44, said labeled peptide comprising the phosphorylatable peptide sequence listed in corresponding Column E, Rows 2-44, of Table 1 (SEQ ID NOs: 1-43), which sequence comprises the phosphorylatable tyrosine listed in corresponding Column D, Rows 2-44, of Table 1.

Among this subset of reagents, antibodies and AQUA peptides for the detection/quantification of the following adaptor/scaffold protein phosphorylation sites are: 14-3-3 zeta (Y82), AKAP2 (Y507), ARRB2 (Y48) and CrkL (48) (see SEQ ID NOs: 1, 8, 26 and 36).

In a second subset of embodiments there is provided:

(i) An isolated phosphorylation site-specific antibody that specifically binds a calcium binding protein selected from Column A, Rows 61-69, of Table 1 only when phosphorylated at the tyrosine listed in corresponding Column D, Rows 61-69, of Table 1, comprised within the phosphorylatable peptide sequence listed in corresponding Column E, Rows 61-69, of Table 1 (SEQ ID NOs: 60-68), wherein said antibody does not bind said protein when not phosphorylated at said tyrosine.
(ii) An equivalent antibody to (i) above that only binds the calcium binding protein when not phosphorylated at the disclosed site (and does not bind the protein when it is phosphorylated at the site).
(iii) A heavy-isotope labeled peptide (AQUA peptide) for the quantification of a signaling protein that is a calcium binding protein selected from Column A, Rows 61-69, said labeled peptide comprising the phosphorylatable peptide sequence listed in corresponding Column E, Rows 61-69, of Table 1 (SEQ ID NOs: 60-68), which sequence comprises the phosphorylatable tyrosine listed in corresponding Column D, Rows 61-69, of Table 1.

Among this subset of reagents, antibodies and AQUA peptides for the detection/quantification of the following calcium binding protein phosphorylation sites are: ANXA11 (Y365), ANXA2 (Y199) and ANXA5 (Y256) (see SEQ ID NOs: 61, 62 and 63).

In another subset of embodiments there is provided:

(i) An isolated phosphorylation site-specific antibody that specifically binds a chromatin or DNA binding/repair/replication protein selected from Column A, Rows 86-96, of Table 1 only when phosphorylated at the tyrosine listed in corresponding Column D, Rows 86-96, of Table 1, comprised within the phosphorylatable peptide sequence listed in corresponding Column E, Rows 86-96, of Table 1 (SEQ ID NOs: 85-95), wherein said antibody does not bind said protein when not phosphorylated at said tyrosine.
(ii) An equivalent antibody to (i) above that only binds the chromatin or DNA binding/repair/replication protein when not phosphorylated at the disclosed site (and does not bind the protein when it is phosphorylated at the site).
(iii) A heavy-isotope labeled peptide (AQUA peptide) for the quantification of a signaling protein that is a chromatin or DNA binding/repair/replication protein selected from Column A, Rows 86-96, said labeled peptide comprising the phosphorylatable peptide sequence listed in corresponding Column E, Rows 86-96, of Table 1 (SEQ ID NOs: 85-95), which sequence comprises the phosphorylatable tyrosine listed in corresponding Column D, Rows 86-96, of Table 1.

Among this subset of reagents, antibodies and AQUA peptides for the detection/quantification of the following chromatin or DNA binding/repair/replication protein phosphorylation sites are: APE1 (Y45) and APTX (Y200) (see SEQ ID NOs: 85 and 87).

In still another subset of embodiments there is provided:

(i) An isolated phosphorylation site-specific antibody that specifically binds a cytoskeletal protein selected from Column A, Rows 97-125, of Table 1 only when phosphorylated at the tyrosine listed in corresponding Column D, Rows 97-125, of Table 1, comprised within the phosphorylatable peptide sequence listed in corresponding Column E, Rows 97-125, of Table 1 (SEQ ID NOs: 96-124), wherein said antibody does not bind said protein when not phosphorylated at said tyrosine.
(ii) An equivalent antibody to (i) above that only binds the cytoskeletal protein when not phosphorylated at the disclosed site (and does not bind the protein when it is phosphorylated at the site).
(iii) A heavy-isotope labeled peptide (AQUA peptide) for the quantification of a signaling protein that is a cytoskeletal protein selected from Column A, Rows 97-125, said labeled peptide comprising the phosphorylatable peptide sequence listed in corresponding Column E, Rows 97-125, of Table 1 (SEQ ID NOs: 96-124), which sequence comprises the phosphorylatable tyrosine listed in corresponding Column D, Rows 97-125, of Table 1.

Among this subset of reagents, antibodies and AQUA peptides for the detection/quantification of the following cytoskeletal protein phosphorylation sites are: ACTN1 (Y582), Arp2 (Y72), Arp3 (Y16), cofilin 1 (Y117), ezrin (Y116) and FLII (Y737) (see SEQ ID NOs: 99, 101, 104, 108, 120 and 124).

In still another subset of embodiments there is provided:

(i) An isolated phosphorylation site-specific antibody that specifically binds an enzyme protein selected from Column A, Rows 130-195, of Table 1 only when phosphorylated at the tyrosine listed in corresponding Column D, Rows 130-195, of Table 1, comprised within the phosphorylatable peptide sequence listed in corresponding Column E, Rows 130-195, of Table 1 (SEQ ID NOs: 129-194), wherein said antibody does not bind said protein when not phosphorylated at said tyrosine.
(ii) An equivalent antibody to (i) above that only binds the enzyme protein when not phosphorylated at the disclosed site (and does not bind the protein when it is phosphorylated at the site).
(iii) A heavy-isotope labeled peptide (AQUA peptide) for the quantification of a signaling protein that is a enzyme protein selected from Column A, Rows 130-195, said labeled peptide comprising the phosphorylatable peptide sequence listed in corresponding Column E, Rows 130-195, of Table 1 (SEQ ID NOs: 129-194), which sequence comprises the phosphorylatable tyrosine listed in corresponding Column D, Rows 130-195, of Table 1.

Among this subset of reagents, antibodies and AQUA peptides for the detection/quantification of the following enzyme protein phosphorylation sites are: ADA (Y67), ASS (Y133), EN02 (Y25) and FASN (Y222) (see SEQ ID NOs: 145, 159, 185 and 194).

In still another subset of embodiments there is provided:

(i) An isolated phosphorylation site-specific antibody that specifically binds a protein kinase (Ser/Thr) selected from Column A, Rows 235-256, of Table 1 only when phosphorylated at the tyrosine listed in corresponding Column D, Rows 235-256, of Table 1, comprised within the phosphorylatable peptide sequence listed in corresponding Column E, Rows 235-256 of Table 1 (SEQ ID NOs: 234-255), wherein said antibody does not bind said protein when not phosphorylated at said tyrosine.
(ii) An equivalent antibody to (i) above that only binds protein kinase (Ser/Thr) when not phosphorylated at the disclosed site (and does not bind the protein when it is phosphorylated at the site).
(iii) A heavy-isotope labeled peptide (AQUA peptide) for the quantification of a signaling protein that is a protein kinase (Ser/Thr) selected from Column A, Rows 235-256, said labeled peptide comprising the phosphorylatable peptide sequence listed in corresponding Column E, Rows 235-256, of Table 1 (SEQ ID NOs: 234-255), which sequence comprises the phosphorylatable tyrosine listed in corresponding Column D, Rows 235-256, of Table 1.

Among this subset of reagents, antibodies and AQUA peptides for the detection/quantification of the following protein kinase (Ser/Thr) phosphorylation sites are: ATM (Y2019), Bcr (Y513), DNA-PK (Y779) and ERK2 (Y36) (see SEQ ID NO: 237, 240, 251 and 253).

In yet another subset of embodiments, there is provided:

(i) An isolated phosphorylation site-specific antibody that specifically binds a protein kinase (Tyr) selected from Column A, Rows 257-267, of Table 1 only when phosphorylated at the tyrosine listed in corresponding Column D, Rows 257-267, of Table 1, comprised within the phosphorylatable peptide sequence listed in corresponding Column E, Rows 257-267, of Table 1 (SEQ ID NOs: 256-266), wherein said antibody does not bind said protein when not phosphorylated at said tyrosine.
(ii) An equivalent antibody to (i) above that only binds the protein kinase (Tyr) when not phosphorylated at the disclosed site (and does not bind the protein when it is phosphorylated at the site).
(iii) A heavy-isotope labeled peptide (AQUA peptide) for the quantification of a signaling protein that is a protein kinase (Tyr) selected from Column A, Rows 257-267, said labeled peptide comprising the phosphorylatable peptide sequence listed in corresponding Column E, Rows 257-267, of Table 1 (SEQ ID NOs: 256-266), which sequence comprises the phosphorylatable tyrosine listed in corresponding Column D, Rows 257-267, of Table 1.

Among this subset of reagents, antibodies and AQUA peptides for the detection/quantification of the following protein kinase (Tyr) phosphorylation sites are: Abl (70), Btk (Y40), CSK (Y416), Fgr (Y208) and FGFR3 (Y577) (see SEQ ID NOs: 256, 258, 259, 261 and 264).

In yet another subset of embodiments, there is provided:

(i) An isolated phosphorylation site-specific antibody that specifically binds a receptor/channel/transporter/cell surface protein selected from Column A, Rows 268-287, of Table 1 only when phosphorylated at the tyrosine listed in corresponding Column D, Rows 268-287, of Table 1, comprised within the phosphorylatable peptide sequence listed in corresponding Column E, Rows 268-287, of Table 1 (SEQ ID NOs: 267-278, 280-287), wherein said antibody does not bind said protein when not phosphorylated at said tyrosine.
(ii) An equivalent antibody to (i) above that only binds the receptor/channel/transporter/cell surface protein when not phosphorylated at the disclosed site (and does not bind the protein when it is phosphorylated at the site).
(iii) A heavy-isotope labeled peptide (AQUA peptide) for the quantification of a signaling protein that is a receptor/channel/transporter/cell surface protein selected from Column A, Rows 268-287, said labeled peptide comprising the phosphorylatable peptide sequence listed in corresponding Column E, Rows 268-287, of Table 1 (SEQ ID NOs: 267-278, 280-287), which sequence comprises the phosphorylatable tyrosine listed in corresponding Column D, Rows 268-287, of Table 1.

Among this subset of reagents, antibodies and AQUA peptides for the detection/quantification of the following receptor/channel/transporter/cell surface protein phosphorylation sites are: CD34 (Y330) and CR2 (Y1029) (see SEQ ID NOs: 280 and 284).

In yet another subset of embodiments, there is provided:

(i) An isolated phosphorylation site-specific antibody that specifically binds a transcriptional regulator selected from Column A, Rows 306-334, of Table 1 only when phosphorylated at the tyrosine listed in corresponding Column D, Rows 306-334, of Table 1, comprised within the phosphorylatable peptide sequence listed in corresponding Column E, Rows 306-334, of Table 1 (SEQ ID NOs: 307-335), wherein said antibody does not bind said protein when not phosphorylated at said tyrosine.
(ii) An equivalent antibody to (i) above that only binds the transcriptional regulator when not phosphorylated at the disclosed site (and does not bind the protein when it is phosphorylated at the site).
(iii) A heavy-isotope labeled peptide (AQUA peptide) for the quantification of a signaling protein that is a transcriptional regulator selected from Column A, Rows 306-334, said labeled peptide comprising the phosphorylatable peptide sequence listed in corresponding Column E, Rows 306-334, of Table 1 (SEQ ID NOs: 307-335), which sequence comprises the phosphorylatable tyrosine listed in corresponding Column D, Rows 306-334, of Table 1.

Among this subset of reagents, antibodies and AQUA peptides for the detection/quantification of the following transcriptional regulator phosphorylation sites are: BAP37 (Y121) and CR2C/EBP-beta (Y137) (see SEQ ID NO: 312 and 318).

In still another subset of embodiments, there is provided:

(i) An isolated phosphorylation site-specific antibody that specifically binds a translational regulator selected from Column A, Rows 335-357, of Table 1 only when phosphorylated at the tyrosine listed in corresponding Column D, Rows 335-357, of Table 1, comprised within the phosphorylatable peptide sequence listed in corresponding Column E, Rows 335-357, of Table 1 (SEQ ID NOs: 336-358), wherein said antibody does not bind said protein when not phosphorylated at said tyrosine.
(ii) An equivalent antibody to (i) above that only binds the translational regulator when not phosphorylated at the disclosed site (and does not bind the protein when it is phosphorylated at the site).
(iii) A heavy-isotope labeled peptide (AQUA peptide) for the quantification of a signaling protein that translational regulator selected from Column A, Rows 335-357, said labeled peptide comprising the phosphorylatable peptide sequence listed in corresponding Column E, Rows 335-357, of Table 1 (SEQ ID NOs: 336-358), which sequence comprises the phosphorylatable tyrosine listed in corresponding Column D, Rows 335-357, of Table 1.

Among this subset of reagents, antibodies and AQUA peptides for the detection/quantification of the following a translational regulator phosphorylation sites are: elF2B (Y298), elF3-eta (Y449), elF3-theta (Y32) and EIF5A (Y97) (see SEQ ID NO: 342, 347, 350 and 358).

In yet a further subset of embodiments, there is provided:

(i) An isolated phosphorylation site-specific antibody that specifically binds a protein selected from the group consisting of catalase (Y83), ACP (Y87), ataxin-3 (Y58), CRMP-2 (Y499) and CLH-17 (Y1205) (Column A, Rows 59, 225, 386, 445 and 491 of Table 1) only when phosphorylated at the tyrosine listed in corresponding Column D of Table 1), said tyrosine comprised within the phosphorylatable peptide sequence listed in corresponding Column E of Table 1 (SEQ ID NOs: 58, 224, 387, 446 and 492), wherein said antibody does not bind said protein when not phosphorylated at said tyrosine.
(ii) An equivalent antibody to (i) above that only binds a protein selected from the group consisting of catalase (Y83), ACP (Y87), ataxin-3 (Y58), CRMP-2 (Y499) and CLH-17 (Y1205) (Column A, Rows 59, 225, 386, 445 and 491 of Table 1) when not phosphorylated at the disclosed site (and does not bind the protein when it is phosphorylated at the site).
(iii) A heavy-isotope labeled peptide (AQUA peptide) for the quantification of a protein selected from the group consisting of catalase (Y83), ACP (Y87), ataxin-3 (Y58), CRMP-2 (Y499) and CLH-17 (Y1205) (Column A, Rows 59, 225, 386, 445 and 491 of Table 1), said labeled peptide comprising the phosphorylatable peptide sequence listed in corresponding Column E of Table 1 (SEQ ID NOs: 58, 224, 387, 446 and 492), which sequence comprises the phosphorylatable tyrosine listed in corresponding Column D, Rows 59, 225, 386, 445 and 491 of Table 1.

The invention also provides an immortalized cell line producing an antibody of the invention, for example, a cell line producing an antibody within any of the foregoing subsets of antibodies. In an embodiment, the immortalized cell line is a rabbit hybridoma or a mouse hybridoma.

In other embodiments, a heavy-isotope labeled peptide (AQUA peptide) of the invention (for example, an AQUA peptide within any of the foregoing subsets of AQUA peptides) comprises a disclosed site sequence wherein the phosphorylatable tyrosine is phosphorylated. In yet other embodiments, a heavy-isotope labeled peptide of the invention comprises a disclosed site sequence wherein the phosphorylatable tyrosine is not phosphorylated.

The foregoing subsets of reagents of the invention should not be construed as limiting the scope of the invention, which, as noted above, includes reagents for the detection and/or quantification of disclosed phosphorylation sites on any of the other protein type/group subsets (each a subset) listed in Column C of Table 1/FIG. 2.

Also provided by the invention are methods for detecting or quantifying a target signaling protein/polypeptide that is tyrosine phosphorylated, said method comprising the step of utilizing one or more of the above-described reagents of the invention to detect or quantify one or more target Signaling Protein(s)/Polypeptide(s) selected from Column A of Table 1 only when phosphorylated at the tyrosine listed in corresponding Column D of Table 1. In certain embodiments of the methods of the invention, the reagents comprise a subset of reagents as described above. The antibodies according to the invention maybe used in standard (e.g., ELISA or conventional cytometric assays). The invention thus, provides compositions and methods for the detection and/or quantitation of a given target signaling protein or polypeptide in a sample, by contacting the sample and a control sample with one or more antibody of the invention under conditions favoring the binding and thus formation of the complex of the antibody with the protein or peptide. The formation of the complex is then detected according to methods well established and known in the art.

Also provided by the invention is a method for obtaining a phosphorylation profile of a certain target protein group, for example adaptor/scaffold proteins or cell cycle regulation proteins (Rows 2-44 and Rows 70-81, respectively, of Table 1), that is phosphorylated in a disease signaling pathway, said method comprising the step of utilizing one or more isolated antibody that specifically binds the protein group selected from Column A of Table 1 only when phosphorylated at the tyrosine listed in corresponding Column D, of Table 1, comprised within the phosphorylation site sequence listed in corresponding Column E, to detect the phosphorylation of one or more of said protein group, thereby obtaining a phosphorylation profile for said protein group.

The invention further contemplates compositions, foremost pharmaceutical compositions, containing one or a more antibody according to the invention formulated together with a pharmaceutically acceptable carrier. One of skill will appreciate that in certain instances the composition of the invention may further comprise other pharmaceutically active moieties. The compounds according to the invention are optionally formulated in a pharmaceutically acceptable vehicle with any of the well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

The invention also provides methods of treating a mammal comprising the step of administering such a mammal a therapeutically effective amount of a composition according to the invention.

As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc. The term “therapeutic composition” refers to any compounds administered to treat or prevent a disease. It will be understood that the subject to which a compound (e.g., an antibody) of the invention is administered need not suffer from a specific traumatic state. Indeed, the compounds (e.g., antibodies) of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

TABLE 1
Phosphorylation Sites

| | A | | | D | E | |
|---|---|---|---|---|---|---|
| | Protein | B | C | Phospho- | Phosphorylation | H |
| 1 | Name | Accession No. | Protein Type | Residue | Site Sequence | SEQ ID NO |

| | | | | | | |
|---|---|---|---|---|---|---|
| 2 | 14-3-3 | NP_003397.1 | Adaptor/scaffold | Y82 | KQQMAREyREKIETELR | SEQ ID NO: 1 |
| | zeta | | | | | |
| 3 | Abi-1 | NP_005461.2 | Adaptor/scaffold | Y484 | NDDGWyEGVCNR | SEQ ID NO: 2 |
| 4 | adaptin, | NP_001273.1 | Adaptor/scaffold | Y136 | CLKDEDPyVR | SEQ ID NO: 3 |
| | beta | | | | | |
| 5 | adaptin, | NP_001273.1 | Adaptor/scaffold | Y888 | NVEGQDMLyQSLK | SEQ ID NO: 4 |
| | beta | | | | | |
| 6 | AFAP | NP_067651.2 | Adaptor/scaffold | Y501 | VISANPYLGGTSNGYAHPSGTALHyDD | SEQ ID NO: 5 |
| | | | | | VPCINGSLK | |
| 7 | AKAP2 | NP_001004065.2 | Adaptor/scaffold | Y939 | WEAGIyANQEEEDNE | SEQ ID NO: 6 |
| 8 | AKAP2 | NP_001004065.2 | Adaptor/scaffold | Y293 | LFEDDEHEKEQyCIR | SEQ ID NO: 7 |
| 9 | AKAP2 | NP_001004065.2 | Adaptor/scaffold | Y507 | SPGALETPSAAGSQGNTASQGKEGPyS | SEQ ID NO: 8 |
| | | | | | EPSKR | |
| 10 | AKAP8 | NP_005849.1 | Adaptor/scaffold | Y229 | SDPFVPPAASSEPLSTPWNELNyVGGR | SEQ ID NO: 9 |
| 11 | AKAP9 | NP_005742.4 | Adaptor/scaffold | Y1233 | CEVNAEDKENSGDyISENEDPELQDYR | SEQ ID NO: 10 |
| 12 | AKAP9 | NP_005742.4 | Adaptor/scaffold | Y2482 | SLENQTyFK | SEQ ID NO: 11 |
| 13 | AKAP9 | NP_005742.4 | Adaptor/scaffold | Y3461 | RILyQNLNEPTTWSLTSDR | SEQ ID NO: 12 |
| 14 | AKAP9 | NP_005742.4 | Adaptor/scaffold | Y66 | HDVSAHHDLNIDQSQCNEMyINSSQR | SEQ ID NO: 13 |
| 15 | AMOTL1 | NP_570899.1 | Adaptor/scaffold | Y219 | GQQQQQQQQGAVGHGYyMAGGTSQK | SEQ ID NO: 14 |
| 16 | ANK1 | NP_000028.3 | Adaptor/scaffold | Y1259 | LRCyCMTDDKVDK | SEQ ID NO: 15 |
| 17 | ANK1 | NP_000028.3 | Adaptor/scaffold | Y3 | PySVGFR | SEQ ID NO: 16 |
| 18 | ANK1 | NP_000028.3 | Adaptor/scaffold | Y100 | ELVNyGANVNAQSQK | SEQ ID NO: 17 |
| 19 | AP1G2 | NP_003908.1 | Adaptor/scaffold | Y87 | VGyLGAMLLLDER | SEQ ID NO: 18 |
| 20 | AP3D1 | NP_003929.4 | Adaptor/scaffold | Y365 | ALDLLyGMVSK | SEQ ID NO: 19 |
| 21 | AP3M1 | NP_036227.1 | Adaptor/scaffold | Y31 | SVVSQSVCDyFFEAQEK | SEQ ID NO: 20 |
| 22 | APBA2 | NP_005494.2 | Adaptor/scaffold | Y193 | EGYQDYYPEEANGNTGASPYR | SEQ ID NO: 21 |
| 23 | APBB1IP | NP_061916.3 | Adaptor/scaffold | Y225 | THCDCNVDWCLYEIyPELQIER | SEQ ID NO: 22 |
| 24 | APBB1IP | NP_061916.3 | Adaptor/scaffold | Y340 | ASGIyYVPK | SEQ ID NO: 23 |
| 25 | APBB1IP | NP_061916.3 | Adaptor/scaffold | Y45 | AEFNySVGFK | SEQ ID NO: 24 |
| 26 | APPL | NP_036228.1 | Adaptor/scaffold | Y522 | SDDHPDWyETMR | SEQ ID NO: 25 |
| 27 | ARRB2 | NP_004304.1 | Adaptor/scaffold | Y48 | DFVDHLDKVDPVDGVVLVDPDyLKDR | SEQ ID NO: 26 |
| 28 | axin 1 | NP_003493.1 | Adaptor/scaffold | Y787 | TRSQRKVGGGSAQPCDSIVVAyYFCGE | SEQ ID NO: 27 |
| | | | | | PIPYR | |
| 29 | axin 1 | NP_003493.1 | Adaptor/scaffold | Y788 | TRSQRKVGGGSAQPCDSIVVAYyFCGE | SEQ ID NO: 28 |
| | | | | | PIPYR | |
| 30 | axin 1 | NP_003493.1 | Adaptor/scaffold | Y796 | TRSQRKVGGGSAQPCDSIVVAYYFCGE | SEQ ID NO: 29 |
| | | | | | PIPyR | |
| 31 | BCAP | NP_689522.2 | Adaptor/scaffold | Y230 | VENEyTISVK | SEQ ID NO: 30 |
| 32 | BIN2 | NP_057377.2 | Adaptor/scaffold | Y232 | LNHNLyEVMSK | SEQ ID NO: 31 |
| 33 | Cas-L | NP_006394.1 | Adaptor/scaffold | Y164 | TGHGyVYEYPSR | SEQ ID NO: 32 |
| 34 | CD2AP | NP_036252.1 | Adaptor/scaffold | Y541 | DTCySPKPSVYLSTPSSASK | SEQ ID NO: 33 |
| 35 | CGN | NP_065821.1 | Adaptor/scaffold | Y51 | ASTyGVAVR | SEQ ID NO: 34 |
| 36 | CrkL | NP_005198.1 | Adaptor/scaffold | Y198 | GKHGNRNSNSyGIPEPAHAY | SEQ ID NO: 35 |
| 37 | CrkL | NP_005198.1 | Adaptor/scaffold | Y48 | DSSTCPGDyVLSVSENSR | SEQ ID NO: 36 |
| 38 | CrkL | NP_005198.1 | Adaptor/scaffold | Y92 | IHyLDTTTLIEPAPR | SEQ ID NO: 37 |
| 39 | diaphanous | NP_005210.3 | Adaptor/scaffold | Y130 | EMVSQYLyTSK | SEQ ID NO: 38 |
| | 1 | | | | | |
| 40 | DNMBP | NP_056036.1 | Adaptor/scaffold | Y515 | HHTSSVySISER | SEQ ID NO: 39 |
| 41 | DOCK2 | NP_004937.1 | Adaptor/scaffold | Y1434 | NKPVPDQIINFyKSNYVQR | SEQ ID NO: 40 |
| 42 | Dok2 | NP_003965.2 | Adaptor/scaffold | Y357 | SLyDSPQEPRGEAW | SEQ ID NO: 41 |
| 43 | FCRL3 | NP_443171.2 | Adaptor/scaffold | Y161 | QLPNSYNLEKITVNSVSRDNSKyHCTA | SEQ ID NO: 42 |
| | | | | | YR | |
| 44 | FCRL3 | NP_443171.2 | Adaptor/scaffold | Y166 | QLPNSYNLEKITVNSVSRDNSKYHCTA | SEQ ID NO: 43 |
| | | | | | yR | |
| 45 | afadin | NP_001035090.1 | Adhesion or | Y1702 | DyEPPSPSPAPGAPPPPPQR | SEQ ID NO: 44 |
| | | | extracellular | | | |
| | | | matrix protein | | | |
| 46 | afadin | NP_005927.2 | Adhesion or | Y568 | VEQQPDyR | SEQ ID NO: 45 |
| | | | extracellular | | | |
| | | | matrix protein | | | |
| 47 | ARHGAP9 | NP_15885.2 | Adhesion or | Y191 | MSEPPVyCNL | SEQ ID NO: 46 |
| | | | extracellular | | | |
| | | | matrix protein | | | |
| 48 | CD93 | NP_036204.2 | Adhesion or | Y644 | AMENQySPTPGTDC | SEQ ID NO: 47 |
| | | | extracellular | | | |
| | | | matrix protein | | | |
| 49 | DNAM-1 | NP_006557.1 | Adhesion or | Y325 | SPISTGQPTNQSMDDTREDIYVNyPTF | SEQ ID NO: 48 |
| | | | extracellular | | SR | |
| | | | matrix protein | | | |
| 50 | FBLN1 | NP_001987.2 | Adhesion or | Y252 | DSSCGTGyELTEDNSCK | SEQ ID NO: 49 |
| | | | extracellular | | | |
| | | | matrix protein | | | |
| 51 | ANKHD1 | NP_060217.1 | Apoptosis | Y1661 | LEGEVTPNSLSTSyK | SEQ ID NO: 50 |
| 52 | Bad | NP_004313.1 | Apoptosis | Y110 | SRSAPPNLWAAQRyGRELRR | SEQ ID NO: 51 |
| 53 | Bag2 | NP_004273.1 | Apoptosis | Y139 | SHLMSLySACSSEVPHGPVDQK | SEQ ID NO: 52 |
| 54 | BAG4 | NP_004865.1 | Apoptosis | Y102 | AGGSHQEQPPYPSYNSNyWNSTAR | SEQ ID NO: 53 |
| 55 | BAG4 | NP_004865.1 | Apoptosis | Y17 | SGYGPSDGPSyGR | SEQ ID NO: 54 |
| 56 | BAG4 | NP_004865.1 | Apoptosis | Y249 | SVPQSGPTVRPQEDAWASPGAyGMGGR | SEQ ID NO: 55 |
| 57 | BAG4 | NP_004865.1 | Apoptosis | Y302 | DSSYPySQSDQSMNR | SEQ ID NO: 56 |
| 58 | BAG4 | NP_004865.1 | Apoptosis | Y9 | SGyGPSDGPSYGR | SEQ ID NO: 57 |
| 59 | catalase | NP_001743.1 | Apoptosis | Y84 | GAGAFGyFEVTHDITK | SEQ ID NO: 58 |
| 60 | DIP | NP_055939.1 | Apoptosis | Y390 | AKYDTPyIIWR | SEQ ID NO: 59 |
| 61 | ANXA11 | NP_001148.1 | Calcium-binding | Y279 | TPVLFDIyEIKEAIK | SEQ ID NO: 60 |
| | | | protein | | | |
| 62 | ANXA11 | NP_001148.1 | Calcium-binding | Y365 | DAQELyAAGENR | SEQ ID NO: 61 |
| | | | protein | | | |
| 63 | ANXA2 | NP_004030.1 | Calcium-binding | Y199 | DLyDAGVKR | SEQ ID NO: 62 |
| | | | protein | | | |
| 64 | ANXA5 | NP_001145.1 | Calcium-binding | Y256 | SIPAYLAETLyYAMK | SEQ ID NO: 63 |
| | | | protein | | | |
| 65 | ANXA5 | NP_001145.1 | Calcium-binding | Y297 | NFATSLySMIK | SEQ ID NO: 64 |
| | | | protein | | | |
| 66 | ANXA6 | NP_004024.2 | Calcium-binding | Y603 | NKPLFFADKLyK | SEQ ID NO: 65 |
| | | | protein | | | |
| 67 | ANXA6 | NP 004024.2 | Calcium-binding | Y95 | LIVGLMRPPAyCDAK | SEQ ID NO: 66 |
| | | | protein | | | |
| 68 | CALB2 | NP_001731.1 | Calcium-binding | Y214 | SGyIDEHELDALLKDLYEK | SEQ ID NO: 67 |
| | | | prorein | | | |
| 69 | calreticulin | NP_004334.1 | Calcium-binding | Y75 | FyALSASFEPFSNK | SEQ ID NO: 68 |
| | | | protein | | | |
| 70 | bamacan | NP_005436.1 | Cell cycle | Y213 | LHTLEEEKEELAQyQKWDK | SEQ ID NO: 69 |
| | | | regulation | | | |
| 71 | bamacan | NP_005436.1 | Cell cycle | Y560 | LFyHIVDSDEVSTK | SEQ ID NO: 70 |
| | | | regulation | | | |
| 72 | bamacan | NP_005436.1 | Cell cycle | Y668 | GALTGGyYDTRK | SEQ ID NO: 71 |
| | | | regulation | | | |
| 73 | Bub3 | NP_004716.1 | Cell cycle | Y141 | TPCNAGTFSQPEKVyTLSVSGDR | SEQ ID NO: 72 |
| | | | regulation | | | |
| 74 | Bub3 | NP_004716.1 | Cell cycle | Y207 | VAVEyLDPSPEVQK | SEQ ID NO: 73 |
| | | | regulation | | | |
| 75 | Cdc23 | NP_004652.2 | Cell cycle | Y353 | AALyFQR | SEQ ID NO: 74 |
| | | | regulation | | | |
| 76 | CENPF | NP_057427.3 | Cell cycle | Y1848 | LEyFSCDHQELLQR | SEQ ID NO: 75 |
| | | | regulation | | | |
| 77 | CEP350 | NP_055625.3 | Cell cycle | Y2572 | ISHIPENFDDyVDINEDEDCYSDER | SEQ ID NO: 76 |
| | | | regulation | | | |
| 78 | CEP350 | NP_055625.3 | Cell cycle | Y2582 | ISHIPENFDDYVDINEDEDCySDER | SEQ ID NO: 77 |
| | | | regulation | | | |
| 79 | CEP350 | NP_055625.3 | Cell cycle | Y2610 | EKDVSEyFYEK | SEQ ID NO: 78 |
| | | | regulation | | | |
| 80 | claspin | NP_071394.2 | Cell cycle | Y887 | NQyQALKPR | SEQ ID NO: 79 |
| | | | regulation | | | |
| 81 | CUL-3 | NP_003581.1 | Cell cycle | Y512 | VLTTGyWPTQSATPK | SEQ ID NO: 80 |
| | | | regulation | | | |
| 82 | CCT7 | NP_006420.1 | Chaperone | Y275 | VHTVEDYQAIVDAEWNILyDKLEK | SEQ ID NO: 81 |
| 83 | CCT- | NP_001753.1 | Chaperone | Y229 | KRVEDAyILTCNVSLEYEK | SEQ ID NO: 82 |
| | zeta | | | | | |
| 84 | CCT- | NP_001753.1 | Chaperone | Y239 | SLEyEKTEVNSG | SEQ ID NO: 83 |
| | zeta | | | | | |
| 85 | FKBP4 | NP_002005.1 | Chaperone | Y411 | EKKLyANMFER | SEQ ID NO: 84 |
| 86 | APE1 | NP_001632.2 | Chromatin, DNA- | Y45 | EAAGEGPALyEDPPDQK | SEQ ID NO: 85 |
| | | | binding, DNA | | | |
| | | | repair or DNA | | | |
| | | | replication protein | | | |
| 87 | APTX | NP_778243.1 | Chromatin, DNA- | Y195 | ISMQDPKMQVYKDEQVVVIKDKyPK | SEQ ID NO: 86 |
| | | | binding, DNA | | | |
| | | | repair or DNA | | | |
| | | | replication protein | | | |
| 88 | APTX | NP_778243.1 | Chromatin, DNA- | Y200 | ARyHWLVLPWTSISSLK | SEQ ID NO: 87 |
| | | | binding, DNA | | | |
| | | | repair or DNA | | | |
| | | | replication protein | | | |
| 89 | ARID1A | NP_006006.3 | Chromatin, DNA- | Y1506 | NDMTyNYANR | SEQ ID NO: 88 |
| | | | binding, DNA | | | |
| | | | repair or DNA | | | |
| | | | replication protein | | | |
| 90 | ARID1A | NP_006006.3 | Chromatin, DNA- | Y1508 | NDMTYNyANR | SEQ ID NO: 89 |
| | | | binding, DNA | | | |
| | | | repair or DNA | | | |
| | | | replication protein | | | |
| 91 | BAF155 | NP_003065.2 | Chromatin, DNA- | Y171 | TLVQNNCLTRPNIyLIPDIDLK | SEQ ID NO: 90 |
| | | | binding, DNA | | | |
| | | | repair or DNA | | | |
| | | | replication protein | | | |
| 92 | BAZ1A | NP_038476.2 | Chromatin, DNA- | Y964 | SSNAyDPSQMCAEK | SEQ ID NO: 91 |
| | | | binding, DNA | | | |
| | | | repair or DNA | | | |
| | | | replication protein | | | |
| 93 | ESCO2 | NP_001017420.1 | Chromatin, DNA- | Y152 | yRHIKPVSRNSR | SEQ ID NO: 92 |
| | | | binding, DNA | | | |
| | | | repair or DNA | | | |
| | | | replication protein | | | |
| 94 | EST1A | NP_060045.4 | Chromatin, DNA- | Y146 | TKKPDLQIyQPGR | SEQ ID NO: 93 |
| | | | binding, DNA | | | |
| | | | repair or DNA | | | |
| | | | replication protein | | | |
| 95 | EST1A | NP_060045.4 | Chromatin, DNA- | Y509 | FQNSDNPYyYPR | SEQ ID NO: 94 |
| | | | binding, DNA | | | |
| | | | repair or DNA | | | |
| | | | replication protein | | | |
| 96 | FBXL10 | NP_115979.3 | Chromatin, DNA- | Y739 | yASNLPGSLLKEQK | SEQ ID NO: 95 |
| | | | binding, DNA | | | |
| | | | repair or DNA | | | |
| | | | replication protein | | | |
| 97 | abLIM | NP_002304.3 | Cytoskeletal | Y383 | VDNEILDyKDLAAIPK | SEQ ID NO: 96 |
| | | | protein | | | |
| 98 | abLIM | NP_002304.3 | Cytoskeletal | Y678 | NGLHRPVSTDFAQYNSyGDVSGGVR | SEQ ID NO: 97 |
| | | | protein | | | |
| 99 | abLIM | NP_002304.3 | Cytoskeletal | Y688 | DyQTLPDGHMPAMR | SEQ ID NO: 98 |
| | | | protein | | | |
| 100 | ACTN1 | NP_001093.1 | Cytoskeletal | Y582 | IVQTyHVNMAGTNPYTTITPQEINGK | SEQ ID NO: 99 |
| | | | protein | | | |
| 101 | ACTN4 | NP_004915.2 | Cytoskeletal | Y693 | ISIEMNGTLEDQLSHLKQyER | SEQ ID NO: 100 |
| | | | protein | | | |
| 102 | ARP2 | NP_005713.1 | Cytoskeletal | Y72 | SMLEVNyPMENGIVR | SEQ ID NO: 101 |
| | | | protein | | | |
| 103 | ARP2 | NP_005713.1 | Cytoskeletal | Y91 | NWDDMKHLWDyTFGPEKLNIDTR | SEQ ID NO: 102 |
| | | | protein | | | |
| 104 | Arp3 | NP_005712.1 | Cytoskeletal | Y100 | yLRAEPEDHYFLLTEPPLNTPENR | SEQ ID NO: 103 |
| | | | protein | | | |
| 105 | Arp3 | NP_005712.1 | Cytoskeletal | Y16 | LPACVVDCGTGyTK | SEQ ID NO: 104 |
| | | | protein | | | |
| 106 | ARPC3 | NP_005710.1 | Cytoskeletal | Y48 | ETKDTDIVDEAIYyFK | SEQ ID NO: 105 |
| | | | protein | | | |
| 107 | CAPZA1 | NP_006126.1 | Cytoskeletal | Y198 | IQVHyYEDGNVQLVSHK | SEQ ID NO: 106 |
| | | | protein | | | |
| 108 | CLIM1 | NP_066272.1 | Cytoskeletal | Y197 | PLDHAQPPSSLVIDKESEVyK | SEQ ID NO: 107 |
| | | | protein | | | |
| 109 | cofilin 1 | NP_005498.1 | Cytoskeletal | Y117 | MIyASSKDAIK | SEQ ID NO: 108 |
| | | | protein | | | |
| 110 | coronin | NP_055140.1 | Cytoskeletal | Y304 | YFEITDESPYVHyLNTFSSKEPQR | SEQ ID NO. 109 |
| | 1C | | protein | | | |
| 111 | DBNL | NP_001014436.1 | Cytoskeletal | Y224 | REQRyQEQGGEASPQR | SEQ ID NO: 110 |
| | | | protein | | | |
| 112 | Destrin | NP_006861.1 | Cytoskeletal | Y117 | MIyASSKDAIK | SEQ ID NO: 111 |
| | | | protein | | | |
| 113 | DST | NP_056363.2 | Cytoskeletal | Y1726 | TLDDIVGRyEDLSK | SEQ ID NO: 112 |
| | | | protein | | | |
| 114 | Emerin | NP_000108.1 | Cytoskeletal | Y41 | IFEyETQR | SEQ ID NO: 113 |
| | | | protein | | | |
| 115 | Emerin | NP_000108.1 | Cytoskeletal | Y59 | LSPPSSSAASSySFSDLNSTR | SEQ ID NO: 114 |
| | | | protein | | | |
| 116 | EPB41 | NP_004428.1 | Cytoskeletal | Y13 | VSLLDDTVyECVVEK | SEQ ID NO: 115 |
| | | | protein | | | |
| 117 | EPB41 | NP_004428.1 | Cytoskeletal | Y525 | TITyEAAQTVK | SEQ ID NO: 116 |
| | iso4 | | protein | | | |
| 118 | EPB41L5 | NP_065960.2 | Cytoskeletal | Y353 | YSGKTEyQTTK | SEQ ID NO: 117 |
| | | | protein | | | |
| 119 | EVL | NP_057421.1 | Cytoskeletal | Y18 | ASVMVyDDTSKK | SEQ ID NO: 118 |
| | | | protein | | | |
| 120 | EVL | NP_057421.1 | Cytoskeletal | Y41 | INIyHNTASNTFR | SEQ ID NO: 119 |
| | | | protein | | | |
| 121 | ezrin | NP_003370.2 | Cytoskeletal | Y116 | EGILSDEIyCPPETAVLLGSYAVQAK | SEQ ID NO: 120 |
| | | | protein | | | |
| 122 | Fascin | NP_003079.1 | Cytoskeletal | Y314 | THTGKyWTLTATGGVQSTASSK | SEQ ID NO: 121 |
| | | | protein | | | |
| 123 | Fascin | NP_003079.1 | Cytoskeletal | Y335 | NASCyFDIEWR | SEQ ID NO: 122 |
| | | | protein | | | |
| 124 | FLII | NP_002009.1 | Cytoskeletal | Y1175 | CSNEKGyFAVTEK | SEQ ID NO: 123 |
| | | | protein | | | |
| 125 | FLII | NP_002009.1 | Cytoskeletal | Y737 | VGLGLGyLELPQINYK | SEQ ID NO: 124 |
| | | | protein | | | |
| 126 | CHERP | NP_006378.3 | Endoplasmic | Y714 | NSEGWEQNGLyEFFR | SEQ ID NO: 125 |
| | | | reticulum or golgi | | | |
| 127 | CHERP | NP_006378.3 | Endoplasmic | Y894 | GVGVALDDPyENYRR | SEQ ID NO: 126 |
| | | | reticulum or golgi | | | |
| 128 | CHERP | NP_006378.3 | Endoplasmic | Y897 | GVGVALDDPYENyRR | SEQ ID NO: 127 |
| | | | reticulum or golgi | | | |
| 129 | FCMD | NP_006722.2 | Endoplasmic | Y31 | HyLSTKNGAGLSK | SEQ ID NO: 128 |
| | | | reticulum or golgi | | | |
| 130 | AARS | NP_001596.2 | Enzyme, misc. | Y192 | DNFWEMGDTGPCGPCSEIHyDR | SEQ ID NO: 129 |
| 131 | AARS | NP_001596.2 | Enzyme, misc. | Y667 | AVyTQDCPLAAAK | SEQ ID NO: 130 |
| 132 | ACADSB | NP_001600.1 | Enzyme, misc. | Y198 | TRADKEGDyYVLNGSK | SEQ ID NO: 131 |
| 133 | ACAT2 | NP_005882.1 | Enzyme, misc. | Y81 | QASVGAGIPySVPAWSCQMICGSGLK | SEQ ID NO: 132 |
| 134 | ACLY | NP_001087.2 | Enzyme, misc. | Y1084 | LKQGLyR | SEQ ID NO: 133 |
| 135 | ACLY | NP_001087.2 | Enzyme, misc. | Y247 | EAyPEEAYIADLDAK | SEQ ID NO: 134 |
| 136 | ACLY | NP_001087.2 | Enzyme, misc. | Y252 | EAYPEEAyIADLDAK | SEQ ID NO: 135 |
| 137 | ACLY | NP_001087.2 | Enzyme, misc. | Y384 | RGGPNyQEGLR | SEQ ID NO: 136 |
| 138 | ACOX1 | NP_004026.2 | Enzyme, misc. | Y275 | LTyGTMVFVR | SEQ ID NO: 137 |
| 139 | ACOX1 | NP_004026.2 | Enzyme, misc. | Y629 | YDGNVyENLFEWAK | SEQ ID NO: 138 |
| 140 | ACSS1 | NP_115890.2 | Enzyme, misc. | Y542 | TEGGyYQITGR | SEQ ID NO: 139 |
| 141 | ACSS2 | NP_061147.1 | Enzyme, misc. | Y561 | DQDGyYWITGR | SEQ ID NO: 140 |
| 142 | ACSS2 | NP_061147.1 | Enzyme, misc. | Y562 | DQDGYyWITGR | SEQ ID NO: 141 |
| 143 | ADA | NP_000013.2 | Enzyme, misc. | Y29 | VELHVHLDGSIKPETILyYGR | SEQ ID NO: 142 |
| 144 | ADA | NP_000013.2 | Enzyme, misc. | Y308 | STLDTDyQMTK | SEQ ID NO: 143 |
| 145 | ADA | NP_000013.2 | Enzyme, misc. | Y348 | ELLDLLyKAYGMPPSASAGQNL | SEQ ID NO: 144 |
| 146 | ADA | NP_000013.2 | Enzyme, misc. | Y67 | FDyYMPAIAGCR | SEQ ID NO: 145 |
| 147 | ADHFE1 | NP_653251.2 | Enzyme, misc | Y334 | SHMHLASAFAGIGFGNAGVHLCHGMSy | SEQ ID NO: 146 |
| | | | | | PISGLVKMYK | |
| 148 | ADHFE1 | NP_653251.2 | Enzyme, misc. | Y343 | SHMHLASAFAGIGFGNAGVHLCHGMSY | SEQ ID NO: 147 |
| | | | | | PISGLVKMyK | |
| 149 | ADSS | NP_001117.2 | Enzyme, misc. | Y170 | KGIGPVySSK | SEQ ID NO: 148 |
| 150 | ADSS | NP_001117.2 | Enzyme, misc. | Y236 | DGVyFLYEALHGPPKK | SEQ ID NO: 149 |
| 151 | ADSS | NP_001117.2 | Enzyme, misc. | Y239 | DGVYFLyEALHGPPKK | SEQ ID NO: 150 |
| 152 | AKR1A1 | NP_006057.1 | Enzyme, misc. | Y210 | GLEVTAySPLGSSDR | SEQ ID NO: 151 |
| 153 | AKR1A1 | NP_006057.1 | Enzyme, misc. | Y50 | HIDCAAIyGNEPEIGEALKEDVGPGK | SEQ ID NO: 152 |
| 154 | ALDH1A1 | NP_000680.2 | Enzyme, misc. | Y426 | ANNTFyGLSAGVFTK | SEQ ID NO: 153 |
| 155 | ALDH1A1 | NP_000680.2 | Enzyme, misc. | Y481 | ELGEyGFHEYTEVK | SEQ ID NO: 154 |
| 156 | ALDH1A2 | NP_003879.2 | Enzyme, misc. | Y168 | YYAGWADKIHGMTIPVDGDyFTFTR | SEQ ID NO: 155 |
| 157 | AMPD2 | NP_004028.3 | Enzyme, misc. | Y145 | SAPyEFPEESPIEQLEER | SEQ ID NO: 156 |
| 158 | APRT | NP_000476.1 | Enzyme, misc. | Y60 | IDyIAGLDSR | SEQ ID NO: 157 |
| 159 | ARD1A | NP_003482.1 | Enzyme, misc. | Y137 | yYADGEDAYAMKR | SEQ ID NO: 158 |
| 160 | ASS | NP_000041.2 | Enzyme, misc. | Y38 | FELSCySLAPQIK | SEQ ID NO: 159 |
| 161 | ASS | NP_000041.2 | Enzyme, misc. | Y322 | FAELVyTGFWHSPECEFVR | SEQ ID NO: 160 |
| 162 | ATIC | NP_004035.2 | Enzyme, misc. | Y64 | LAGDKANyWWLR | SEQ ID NO: 161 |
| 163 | autotaxin | N_P006200.3 | Enzyme, misc. | Y514 | WHVARKPLDVyKK | SEQ ID NO: 162 |
| 164 | BCKDHB | NP_000047.1 | Enzyme, misc. | Y383 | WKCyDALRKMINY | SEQ ID NO: 163 |
| 165 | BCKDHB | NP_000047.1 | Enzyme, misc. | Y392 | WKCYDALRKMINy | SEQ ID NO: 164 |
| 166 | CBR3 | NP_001227.1 | Enzyme, misc. | Y194 | EGWPNSPyGVSK | SEQ ID NO: 165 |
| 167 | COX5A | NP_004246.1 | Enzyme, misc. | Y80 | GINTLVTyDMVPEPK | SEQ ID NO: 166 |
| 168 | COX7A2L | NP_004709.2 | Enzyme, misc. | Y52 | LTSDSTVyDYAGK | SEQ ID NO: 167 |
| 169 | CSS3 | NP_787052.3 | Enzyme, misc. | Y526 | EIQYGyRRVNPMHGVEYILDLLLLYKR | SEQ ID NO: 168 |
| | | | | | HK | |
| 170 | CysRS | NP_001742.1 | Enzyme, misc. | Y32C | LHLyNSLTR | SEQ ID NO: 169 |
| 171 | DARS | NP_001340.2 | Enzyme, misc. | Y245 | NNAyLAQSPQLYK | SEQ ID NO: 170 |
| 172 | DDX18 | NP_006764.3 | Enzyme, misc. | Y440 | yHYELLNYIDLPVLAIHGK | SEQ ID NO: 171 |
| 173 | DDX18 | NP_006764.3 | Enzyme, misc. | Y442 | YHyELLNYIDLPVLAIHGK | SEQ ID NO: 172 |
| 174 | DDX18 | NP_006764.3 | Enzyme, misc. | Y447 | YHYELLNyIDLPVLAIHGK | SEQ ID NO: 173 |
| 175 | DDX9 | NP_001348.2 | Enzyme, misc. | Y148 | GANLKDyYSR | SEQ ID NO: 174 |
| 176 | DDX9 | NP_001348.2 | Enzyme, misc. | Y259 | QLYHLGVVEAySGLTK | SEQ 10 NO: 175 |
| 177 | DDX9 | NP_001348.2 | Enzyme, misc. | Y68 | DFVNyLVR | SEQ ID NO: 176 |
| 178 | DHX15 | NP_001349.2 | Enzyme, misc. | Y499 | TEMQDNTyPEILR | SEQ ID NO: 177 |
| 179 | DHX36 | NP_065916.1 | Enzyme, misc. | Y1007 | FQDGYyS | SEQ ID NO: 178 |
| 180 | DHX36 | NP_065916.1 | Enzyme, misc. | Y626 | ASLLDDyQLPEILR | SEQ ID NO: 179 |
| 181 | Dicer1 | NP_085124.2 | Enzyme, misc. | Y1330 | HAITTyLFCTYPDAHEGR | SEQ ID NO: 180 |
| 182 | DP103 | NP_009135.3 | Enzyme, misc. | Y616 | HyTGPGDQTVNPQNGFVR | SEQ ID NO: 181 |
| 183 | DPM1 | NP_03850.1 | Enzyme, misc. | Y113 | HATGNyIIIMDADLSHHPK | SEQ ID NO: 182 |
| 184 | ELP3 | NP_060561.3 | Enzyme, misc. | Y207 | NLHDALSGHTSNNIYEAVKySER | SEQ ID NO: 183 |
| 185 | ELP3 | NP_060561.3 | Enzyme, misc. | Y329 | GTGLyELWK | SEQ ID NO: 184 |
| 186 | ENO2 | NP_001966.1 | Enzyme, misc. | Y25 | GNPTVEVDLyTAK | SEQ ID NO: 185 |
| 187 | EPRS | NP_004437.2 | Enzyme, misc. | Y1349 | DNySPGWK | SEQ ID NO: 186 |
| 188 | EPRS | NP_004437.2 | Enzyme, misc. | Y399 | TTEyHDRDEQFYWIIEALGIR | SEQ ID NO: 187 |
| 189 | EPRS | NP_004437.2 | Enzyme, misc. | Y407 | TTEYHDRDEQFyWIIEALGIR | SEQ ID NO: 188 |
| 190 | EPRS | NP_004437.2 | Enzyme, misc. | Y827 | SLyDEVAAQGEVVRK | SEQ ID NO: 189 |
| 191 | EZH2 | NP_004447.2 | Enzyme, misc. | Y701 | FANHSVNPNCyAK | SEQ ID NO: 190 |
| 192 | F13A1 | NP_000120.2 | Enzyme, misc. | Y104 | VEyVIGRYPQENK | SEQ ID NO: 191 |
| 193 | F13A1 | NP_000120.2 | Enzyme, misc. | Y109 | VEYVIGRyPQENK | SEQ ID NO: 192 |
| 194 | FARSLA | NP_004452.1 | Enzyme, misc. | Y488 | VNLQMVyDSPLCR | SEQ ID NO: 193 |
| 195 | FASN | NP_004095.4 | Enzyme, misc. | Y222 | AFDTAGNGyCR | SEQ ID NO: 194 |
| 196 | ABR | NP_001083.2 | G protein or | Y227 | HTPVDHPDyPLLQDALR | SEQ ID NO: 195 |
| | | | regulator | | | |
| 197 | ARAP3 | NP_071926.4 | G protein or | Y679 | ATySGFLYCSPVSNK | SEQ ID NO: 196 |
| | | | regulator | | | |
| 198 | ARF | CAI20950.1 | G protein or | Y356 | KKYNDDSDDSYFTSSSSyFDEPVELR | SEQ ID NO: 197 |
| | GAP 3 | | regulator | | | |
| 199 | ARF | NP_055385.2 | G protein or | Y408 | RKPDyEPVENTDEAQKK | SEQ ID NO: 198 |
| | GAP 3 | | regulator | | | |
| 200 | ARF | NP_055385.2 | G protein or | Y432 | AISSDMyFGR | SEQ ID NO: 199 |
| | GAP 3 | | regulator | | | |
| 201 | ARF | NP_060679.1 | G protein or | Y175 | VSGQPQSVTASSDKAFEDWLNDDLGSy | SEQ ID NO. 200 |
| | GAP1 | | regulator | | QGAQGNR | |
| 202 | ARHGEF6 | NP_004831.1 | G protein or | Y719 | SLVDTVyALKDEVR | SEQ ID NO: 201 |
| | | | regulator | | | |
| 203 | centaurin- | NP_056057.1 | G protein or | Y264 | SFDLTTPyR | SEQ ID NO: 202 |
| | delta 2 | | regulator | | | |
| 204 | centaurin- | NP_056057.1 | G protein or | Y543 | WCVLGDGVLSyFENER | SEQ ID NO: 203 |
| | delta 2 | | regulator | | | |
| 205 | ECT2 | NP_060568.3 | G protein or | Y213 | VAVSLGTPIMKPEWIyKAWER | SEQ ID NO: 204 |
| | | | regulator | | | |
| 206 | ARPP-21 | NP_057384.2 | Inhibitor protein | Y288 | SIEEREEEyQRVR | SEQ ID NO: 205 |
| 207 | calpastatin | NP_001741.4 | Inhibitor protein | Y422 | CGEDDETIPSEyR | SEQ ID NO: 206 |
| 208 | AK2 | NP_001616.1 | Kinase (non- | Y12 | APSVPAAEPEyPK | SEQ ID NO: 207 |
| | | | protein) | | | |
| 209 | AK2 | NP_001616.1 | Kinase (non- | Y201 | LQAYHTQTTPLIEYyR | SEQ ID NO: 208 |
| | | | protein) | | | |
| 210 | endofin | NP_055548.2 | Lipid binding | Y73 | DQECVNSCASSETSyGTNESSLNEK | SEQ ID NO: 209 |
| | | | protein | | | |
| 211 | ATP5O | NP_001688.1 | Mitochondrial | Y35 | LVRPPVQVyGIEGR | SEQ ID NO: 210 |
| | | | protein | | | |
| 212 | DNAH5 | NP_001360.1 | Motor or | Y1600 | yNMPFKAQIQK | SEQ ID NO: 211 |
| | | | contractile protein | | | |
| 213 | DNAH8 | NP_001362.2 | Motor or | Y3631 | MKELEDNLLyK | SEQ ID NO: 212 |
| | | | contractile protein | | | |
| 214 | DNCH1 | NP_001367.2 | Motor or | Y1010 | YQVGVHyELTEEEKFYR | SEQ ID NO: 213 |
| | | | contractile protein | | | |
| 215 | DNCH1 | NP_001367.2 | Motor or | Y1283 | TKPVTGNLRPEEALQALTIyEGKFGR | SEQ ID NO: 214 |
| | | | contractile protein | | | |
| 216 | DNCH1 | NP_001367.2 | Motor or | Y3103 | CVLNWFGDWSTEALyQVGK | SEQ ID NO: 215 |
| | | | contractile protein | | | |
| 217 | DNCH1 | NP_001367.2 | Motor or | Y3377 | NYMSNPSyNYEIVNR | SEQ ID NO: 216 |
| | | | contractile protein | | | |
| 218 | dynactin1 | NP_004073.2 | Motor or | Y1149 | LSHEGPGSELPAGALyR | SEQ ID NO: 217 |
| | | | contractile protein | | | |
| 219 | dynactin1 | NP_004073.2 | Motor or | Y1244 | AKEEQQDDTVyMGK | SEQ ID NO: 218 |
| | | | contractile protein | | | |
| 220 | dynactin2 | NP_006391.1 | Motor or | Y318 | VHQLyETIQR | SEQ ID NO: 219 |
| | | | contractile protein | | | |
| 221 | dynactin4 | NP_057305.1 | Motor or | Y107 | KAyYLACGFCR | SEQ ID NO: 220 |
| | | | contractile protein | | | |
| 222 | dynactin4 | NP_057305.1 | Motor or | Y108 | KAYyLACGFCR | SEQ ID NO: 221 |
| | | | contractile protein | | | |
| 223 | Eg5 | NP_004514.2 | Motor or | Y211 | GLEEITVHNKDEVyQILEK | SEQ ID NO: 222 |
| | | | contractile protein | | | |
| 224 | Eg5 | NP_004514.2 | Motor or | Y399 | NGVyISEENFR | SEQ ID NO: 223 |
| | | | contractile protein | | | |
| 225 | ACP1 | NP_004291.1 | Phosphatase | Y88 | QITKEDFATFDyILCMDESNLR | SEQ ID NO: 224 |
| 226 | DUSP7 | NP_001938.1 | Phosphatase | Y118 | LRDDGCQAyYLQGGFNK | SEQ ID NO: 225 |
| 227 | APEH | NP_001631.3 | Protease | Y209 | LKKPDQAIKGDQFVFyEDWGENMVSK | SEQ ID NO: 226 |
| 228 | calpain 2 | NP_001739.1 | Protease | Y377 | GSTAGGCRNyPNTFWMNPQYLIK | SEQ ID NO: 227 |
| 229 | CAPN1 | NP_005177.2 | Protease | Y371 | KWNTTLyEGTWR | SEQ ID NO: 228 |
| 230 | CAPN1 | NP_005177.2 | Protease | Y387 | GSTAGGCRNyPATFWVNPQFK | SEQ ID NO: 229 |
| 231 | CASP10 | NP_001221.2 | Protease | Y9 | SQGQHWySSSDKNCK | SEQ ID NO: 230 |
| 232 | Bub1 | NP_004327.1 | protein kinase | Y219 | SEySVHSSLASK | SEQ ID NO: 231 |
| 233 | DYRK1A | NP_001387.2 | protein kinase, | Y111 | HINEVyYAK | SEQ ID NO: 232 |
| | | | dual-specificity | | | |
| 234 | DYRK1A | NP_001387.2 | protein kinase, | Y147 | VYNDGYDDDNYDyIVK | SEQ ID NO: 233 |
| | | | dual-specificity | | | |
| 235 | AAK1 | NP_055726.2 | protein kinase, | Y34 | REQGGSGLGSGSSGGGGSTSGLGSGyI | SEQ ID NO: 234 |
| | | | Ser/Thr (non- | | GR | |
| | | | receptor) | | | |
| 236 | ATM | NP_000042.3 | protein kinase, | Y1753 | TGHSFWEIyKMTTDPMLAYLQPFRTSR | SEQ ID NO: 235 |
| | | | Ser/Thr (non- | | | |
| | | | receptor) | | | |
| 237 | ATM | NP_000042.3 | protein kinase, | Y1763 | TGHSFWEIYKMTTDPMLAyLQPFRTSR | SEQ ID NO: 236 |
| | | | Ser/Thr (non- | | | |
| | | | receptor) | | | |
| 238 | ATM | NP_000042.3 | protein kinase, | Y2019 | SIGEPDSLyGCGGGK | SEQ ID NO: 237 |
| | | | Ser/Thr (non- | | | |
| | | | receptor) | | | |
| 239 | Bcr | NP_004318.3 | protein kinase, | Y279 | DNLIDANGGSRPPWPPLEYQPyQSIYV | SEQ ID NO: 238 |
| | | | Ser/Thr (non- | | GGMMEGEGK | |
| | | | receptor) | | | |
| 240 | Bcr | NP_004318.3 | protein kinase, | Y455 | HQDGLPyIDDSPSSSPHLSSK | SEQ ID NO. 239 |
| | | | Ser/Thr (non- | | | |
| | | | receptor) | | | |
| 241 | Bcr | NP_004318.3 | protein kinase, | Y513 | KWVLSGILASEETyLSHLEALLLPMKP | SEQ ID NO: 240 |
| | | | Ser/Thr (non- | | LK | |
| | | | receptor) | | | |
| 242 | BIKE | NP_942595.1 | protein kinase, | Y813 | ySDMSSVYR | SEQ ID NO: 241 |
| | | | Ser/Thr (non- | | | |
| | | | receptor) | | | |
| 243 | BIKE | NP_942595.1 | protein kinase, | Y820 | YSDMSSVyR | SEQ ID NO: 242 |
| | | | Ser/Thr (non- | | | |
| | | | receptor) | | | |
| 244 | CAMKK2 | NP_006540.3 | protein kinase, | Y234 | GPIEQVyQEIAILK | SEQ ID NO: 243 |
| | | | Ser/Thr (non- | | | |
| | | | receptor) | | | |
| 245 | CDK3 | NP_001249.1 | protein kinase, | Y19 | IGEGTYGVVyKAK | SEQ ID NO: 244 |
| | | | Ser/Thr (non- | | | |
| | | | receptor) | | | |
| 246 | CK1-A | CAA56710.1 | protein kinase, | Y17 | yKLVRKIGSGSFGDIYLAINITNGEEV | SEQ ID NO. 245 |
| | | | Ser/Thr (non- | | ALKLESQK | |
| | | | receptor) | | | |
| 247 | CK1-A | CAA56710.1 | protein kinase, | Y32 | YKLVRKIGSGSFGDIyLAINITNGEEV | SEQ ID NO: 246 |
| | | | Ser/Thr (non- | | ALKLESQK | |
| | | | receptor) | | | |
| 248 | CK2- | NP_001887.1 | protein kinase, | Y13 | ARVyAEVNSLR | SEQ ID NO: 247 |
| | alpha2 | | Ser/Thr (non- | | | |
| | | | receptor) | | | |
| 249 | CPNE3 | NP_003900.1 | protein kinase, | Y203 | ISLNSLCyGDMDK | SEQ ID NO: 248 |
| | | | Ser/Thr (non- | | | |
| | | | receptor) | | | |
| 250 | CRIK | NP_009105.1 | protein kinase, | Y1417 | CSTCLPATCGLPAEyATHFTEAFCR | SEQ ID NO: 249 |
| | | | Ser/Thr (non- | | | |
| | | | receptor) | | | |
| 251 | DNA-PK | NP_008835.5 | protein kinase, | Y2936 | SIGEyDVLR | SEQ ID NO: 250 |
| | | | Ser/Thr (non- | | | |
| | | | receptor) | | | |
| 252 | DNA-PK | NP_008835.5 | protein kinase, | Y779 | SIyIDRHVMQPY | SEQ ID NO: 251 |
| | | | Ser/Thr (non- | | | |
| | | | receptor) | | | |
| 253 | DNA-PK | NP_008835.5 | protein kinase, | Y883 | SyVAWDREK | SEQ ID NO: 252 |
| | | | Ser/Thr (non- | | | |
| | | | receptor) | | | |
| 254 | ERK2 | NP_002736.3 | protein kinase, | Y36 | YTNLSYIGEGAyGMVCSAYDNVNKVR | SEQ ID NO. 253 |
| | | | Ser/Thr (non- | | | |
| | | | receptor) | | | |
| 255 | ERK2 | NP_002736.3 | protein kinase, | Y43 | YTNLSYIGEGAYGMVCSAyDNVNKVR | SEQ ID NO: 254 |
| | | | Ser/Thr (non- | | | |
| | | | receptor) | | | |
| 256 | ERK3 | NP_002739.1 | protein kinase, | Y209 | LLLSPNNyTK | SEQ ID NO: 255 |
| | | | Ser/Thr (non- | | | |
| | | | receptor) | | | |
| 257 | Abl | NP_005148.2 | protein kinase, | Y70 | ENLLAGPSENDPNLFVALyDFVASGON | SEQ ID NO. 256 |
| | | | Tyr (non- | | TLSITK | |
| | | | receptor) | | | |
| 258 | Btk | NP_000052.1 | protein kinase, | Y334 | STGDPQGVIRHyVVCSTPQSQYYLAEK | SEQ ID NO: 257 |
| | | | Tyr (non- | | | |
| | | | receptor) | | | |
| 259 | Btk | NP_000052.1 | protein kinase, | Y40 | LSYyEYDFER | SEQ ID NO: 258 |
| | | | Tyr (non- | | | |
| | | | receptor) | | | |
| 260 | CSK | NP_004374.1 | protein kinase, | Y416 | MDAPDGCPPAVyEVMK | SEQ ID NO: 259 |
| | | | Tyr (non- | | | |
| | | | receptor) | | | |
| 261 | Fer | NP_005237.2 | protein kinase, | Y734 | WTAPEALNyGR | SEQ ID NO: 260 |
| | | | Tyr (non- | | | |
| | | | receptor) | | | |
| 262 | Fgr | NP_005239.1 | protein kinase, | Y208 | KLDMGGyYITTR | SEQ ID NO: 261 |
| | | | Tyr (non- | | | |
| | | | receptor) | | | |
| 263 | Fgr | NP_005239.1 | protein kinase, | Y209 | KLDMGGYyITTR | SEQ ID NO: 262 |
| | | | Tyr (non- | | | |
| | | | receptor) | | | |
| 264 | FGFR3 | NP_000133.1 | protein kinase, | Y552 | HKNIINLLGACTQGGPLyVLVEYAAK | SEQ ID NO: 263 |
| | | | Tyr (receptor) | | | |
| 265 | FGFR3 | NP_000133.1 | protein kinase, | Y577 | RPPGLDySFDTCKPPEEQLTFK | SEQ ID NO: 264 |
| | | | Tyr (receptor) | | | |
| 266 | FGFR3 | NP_000133.1 | protein kinase, | Y647 | DVHNLDyYKKTTNGRLPVK | SEQ ID NO: 265 |
| | | | Tyr (receptor) | | | |
| 267 | FGFR3 | NP_000133.1 | protein kinase, | Y648 | DVHNLDYyKKTTNGRLPVK | SEQ ID NO: 266 |
| | | | Tyr (receptor) | | | |
| 268 | ABCC4 | NP_005836.1 | Receptor, | Y1255 | EyDEPYVLLQNK | SEQ ID NO: 267 |
| | | | channel, | | | |
| | | | transporter or cell | | | |
| | | | surface protein | | | |
| 269 | ABCC4 | NP_005836.1 | Receptor, | Y1259 | EYDEPyVLLQNK | SEQ ID NO: 268 |
| | | | channel, | | | |
| | | | transporter or cell | | | |
| | | | surface protein | | | |
| 270 | ATP5H | NP_006347.1 | Receptor, | Y150 | yPYWPHQPIENL | SEQ ID NO: 269 |
| | | | channel, | | | |
| | | | transporter or cell | | | |
| | | | surface protein | | | |
| 271 | ATP5H | NP_006347.1 | Receptor, | Y152 | YPyWPHQPIENL | SEQ ID NO: 270 |
| | | | channel, | | | |
| | | | transporter or cell | | | |
| | | | surface protein | | | |
| 272 | ATP5H | NP_006347.1 | Receptor, | Y56 | LAALPENPPAIDWAyYK | SEQ ID NO: 271 |
| | | | channel, | | | |
| | | | transporter or cell | | | |
| | | | surface protein | | | |
| 273 | ATP6V1A | NP_001681.2 | Receptor, | Y465 | ALDEYyDKHFTEFVPLR | SEQ ID NO: 272 |
| | | | channel, | | | |
| | | | transporter or cell | | | |
| | | | surface protein | | | |
| 274 | ATP6V1 | NP_001686.1 | Receptor, | Y367 | HLDSSAAAIIDAPMDIPGLNLSQQEYy | SEQ ID NO: 273 |
| | C1 | | channel, | | PYVYYK | |
| | | | transporter or cell | | | |
| | | | surface protein | | | |
| 275 | ATP6V1 | NP_001687.1 | Receptor, | Y56 | IMEyYEK | SEQ ID NO: 274 |
| | E1 | | channel, | | | |
| | | | transporter or cell | | | |
| | | | surface protein | | | |
| 276 | ATP6V1H | NP_057025.2 | Receptor, | Y388 | LNEKNyELLK | SEQ ID NO: 275 |
| | | | channel, | | | |
| | | | transporter or cell | | | |
| | | | surface protein | | | |
| 277 | ATP8B1 | NP_005594.1 | Receptor, | Y1217 | GyADLISSGR | SEQ ID NO: 276 |
| | | | channel, | | | |
| | | | transporter or cell | | | |
| | | | surface protein | | | |
| 278 | BAI2 | NP_001694.2 | Receptor, | Y655 | ATyVPSADDVQR | SEQ ID NO: 277 |
| | | | channel, | | | |
| | | | transporter or cell | | | |
| | | | surface protein | | | |
| 279 | BTN3A3 | NP_008925.1 | Receptor, | Y295 | EREMKEMGyAATEQEISLREKLQEELK | SEQ ID NO: 278 |
| | | | channel, | | | |
| | | | transporter or cell | | | |
| | | | surface protein | | | |
| 280 | CD34 | NP_001020280.1 | Receptor, | Y330 | LGEDPYyTENGGGQGYSSGPGTSPEAQ | SEQ ID NO: 280 |
| | | | channel, | | GK | |
| | | | transporter or cell | | | |
| | | | surface protein | | | |
| 281 | CD7 | NP_006128.1 | Receptor, | Y222 | DKNSAACVVyEDMSHSR | SEQ ID NO: 281 |
| | | | channel, | | | |
| | | | transporter or cell | | | |
| | | | surface protein | | | |
| 282 | CD84 | NP_003865.1 | Receptor, | Y264 | TIYTyIMASR | SEQ ID NO: 282 |
| | | | channel, | | | |
| | | | transporter or cell | | | |
| | | | surface protein | | | |
| 283 | CD84 | NP_003865.1 | Receptor, | Y324 | ASTQDSKPPGTSSyEIVI | SEQ ID NO: 283 |
| | | | channel, | | | |
| | | | transporter or cell | | | |
| | | | surface protein | | | |
| 284 | CR2 | NP_001868.2 | Receptor, | Y1029 | EVYSV0PyNPAS | SEQ ID NO: 284 |
| | | | channel, | | | |
| | | | transporter or cell | | | |
| | | | surface protein | | | |
| 285 | eIF4ENI | NP_062817.1 | Receptor, | Y580 | AASADyLRPR | SEQ ID NO: 285 |
| | F1 | | channel, | | | |
| | | | transporter or cell | | | |
| | | | surface protein | | | |
| 286 | ETB | NP_000106.1 | Receptor, | Y430 | FKANDHGyDNFR | SEQ ID NO: 286 |
| | | | channel, | | | |
| | | | transporter or cell | | | |
| | | | surface protein | | | |
| 287 | FCRL5 | NP_112571.1 | Receptor, | Y924 | GENVVySEVR | SEQ ID NO: 287 |
| | | | channel, | | | |
| | | | transporter or cell | | | |
| | | | surface protein | | | |
| 288 | CPSF6 | NP_008938.1 | RNA binding | Y384 | GPPPT0PyGRPPPYDR | SEQ ID NO: 288 |
| | | | protein | | | |
| 289 | CPSF6 | NP_008938.1 | RNA binding | Y74 | GAAPNVVyTYTGKR | SEQ ID NO: 289 |
| | | | protein | | | |
| 290 | CstF-50 | NP_001315.1 | RNA binding | Y195 | TLYDHVDEVTCLAFHPTEQILASGSR | SEQ ID NO: 291 |
| | | | protein | | DyTLK | |
| 291 | CstF-50 | NP_001315.1 | RNA binding | Y67 | LGMENDDTAVQyAIGR | SEQ ID NO: 292 |
| | | | protein | | | |
| 292 | CstF-77 | NP_001317.1 | RNA binding | Y534 | FMDLyPCSASELK | SEQ ID NO: 293 |
| | | | protein | | | |
| 293 | CUGBP2 | NP_006552.3 | RNA binding | Y70 | ELFEPyGAVYQINVLR | SEQ ID NO: 294 |
| | | | protein | | | |
| 294 | DAZ1 | NP_004072.2 | RNA binding | Y515 | QAFPAYPSSPFQVTTGyQLPVYNY | SEQ ID NO: 295 |
| | | | protein | | | |
| 295 | DAZ1 | NP_004072.2 | RNA binding | Y520 | QAFPAYPSSPFQVTTGYQLPVyNY | SEQ ID NO: 296 |
| | | | protein | | | |
| 296 | DAZ1 | NP_004072.2 | RNA binding | Y522 | QAFPAYPSSPFQVTTGYQLPVYNy | SEQ ID NO: 297 |
| | | | protein | | | |
| 297 | DDX23 | NP_004809.2 | RNA binding | Y470 | IDRIEESDQGPyAIILAPTR | SEQ ID NO: 298 |
| | | | protein | | | |
| 298 | DDX3 | NP_001347.3 | RNA binding | Y301 | VRPCVVyGGADIGQQIR | SEQ ID NO: 299 |
| | | | protein | | | |
| 299 | DDX3 | NP_001347.3 | RNA binding | Y53 | GFyDK0SSGWSSSKDKDAYSSFGSR | SEQ ID NO: 300 |
| | | | protein | | | |
| 300 | DDX5 | NP_004387.1 | RNA binding | Y518 | DRENYDRGySSLLKR | SEQ ID NO: 301 |
| | | | protein | | | |
| 301 | DDX5 | NP_004387.1 | RNA binding | Y97 | GHNCPKPVLNFyEANFPANVMDVIAR | SEQ ID NO: 302 |
| | | | protein | | | |
| 302 | dyskerin | NP_001354.1 | RNA binding | Y153 | SQQSAGKEyVGIVR | SEQ ID NO: 303 |
| | | | protein | | | |
| 303 | E1B-AP5 | NP_008971.2 | RNA binding | Y510 | NYILDQTNVyGSAQRR | SEQ ID NO: 304 |
| | | | protein | | | |
| 304 | ELAVL1 | NP_001410.2 | RNA binding | Y109 | VSYARPSSEVIKDANLyISGLPR | SEQ ID NO: 305 |
| | | | protein | | | |
| 305 | FAM51A1 | NP_060326.1 | RNA binding | Y179 | LDSYVNADHDLyCNTR | SEQ ID NO: 306 |
| | | | protein | | | |
| 306 | 53BP1 | NP_005648.1 | Transcriptional | Y522 | LMLSTSEySQSPK | SEQ ID NO: 307 |
| | | | regulator | | | |
| 307 | AF-4 | NP_005926.1 | Transcriptional | Y64 | IQNMLGNyEEVK | SEQ ID NO: 308 |
| | | | regulator | | | |
| 308 | AIP | NP_003968.1 | Transcriptional | Y247 | LVVEEyYEVLDHCSSILNK | SEQ ID NO: 309 |
| | | | regulator | | | |
| 309 | AKNA | NP_110394.2 | Transcriptional | Y356 | GQLNyPLPDFSK | SEQ ID NO: 310 |
| | | | regulator | | | |
| 310 | ASCC3 | NP_006819.2 | Transcriptional | Y975 | TGyFSSTDLGR | SEQ ID NO: 311 |
| | | | regulator | | | |
| 311 | BAP37 | NP_009204.1 | Transcriptional | Y121 | VLSRPNAQELPSMyQR | SEQ ID NO: 312 |
| | | | regulator | | | |
| 312 | BAP37 | NP_009204.1 | Transcriptional | Y34 | LLLGAGAVAyGVR | SEQ ID NO: 313 |
| | | | regulator | | | |
| 313 | BAP37 | NP_009204.1 | Transcriptional | Y81 | IPWFQYPIIyDIR | SEQ ID NO: 314 |
| | | | regulator | | | |
| 314 | BCoR | NP_060215.4 | Transcriptional | Y972 | LAKRIANSAGyVGDR | SEQ ID NO: 315 |
| | | | regulator | | | |
| 315 | Btf | NP_055554.1 | Transcriptional | Y408 | FNDSEGDDTEETEDyR | SEQ ID NO: 316 |
| | | | regulator | | | |
| 316 | Btf | NP_055554.1 | Transcriptional | Y786 | EySGFAGVSR | SEQ ID NO: 317 |
| | | | regulator | | | |
| 317 | C/EBP- | NP_005185.2 | Transcriptional | Y137 | KPAEyGYVSLGR | SEQ ID NO: 318 |
| | beta | | regulator | | | |
| 318 | C/EBP- | NP_001796.2 | Transcriptional | Y107 | ALGPGIySSPGSYDPR | SEQ ID NO: 319 |
| | epsilon | | regulator | | | |
| 319 | CRSP6 | NP_004259.3 | Transcriptional | Y256 | NTDLDLDKKIPEDYCPLDVQIPSDLE | SEQ ID NO: 320 |
| | | | regulator | | GSAyIK | |
| 320 | CTBP2 | NP_001320.1 | Transcriptional | Y108 | IGSGyDNVDIK | SEQ ID NO: 321 |
| | | | regulator | | | |
| 321 | Daxx | NP_001341.1 | Transcriptional | Y124 | PAKLyVYINELCTVLK | SEQ ID NO: 322 |
| | | | regulator | | | |
| 322 | Daxx | NP_001341.1 | Transcriptional | Y126 | PAKLYVyINELCTVLK | SEQ ID NO: 323 |
| | | | regulator | | | |
| 323 | DDX17 | NP_006377.2 | Transcriptional | Y75 | APLPDLyPFGTMR | SEQ ID NO: 324 |
| | | | regulator | | | |
| 324 | DTX2 | NP_065943.1 | Transcriptional | Y156 | GNQLVDLAPLGYNYTVNyTTHTQTNK | SEQ ID NO: 325 |
| | | | regulator | | | |
| 325 | EDG2 | NP_003901.2 | Transcriptional | Y97 | KQGyENLCCLR | SEQ ID NO: 326 |
| | | | regulator | | | |
| 326 | elongin | NP_005639.1 | Transcriptional | Y18 | TYGGCEGPDAMyVK | SEQ ID NO: 327 |
| | C | | regulator | | | |
| 327 | ERG | NP_004440.1 | Transcriptional | Y276 | TEDQRPQLDPyQILGPTSSR | SEQ ID NO: 328 |
| | | | regulator | | | |
| 328 | ERG | NP_004440.1 | Transcriptional | Y345 | SKPNMNyDKLSR | SEQ ID NO: 329 |
| | | | regulator | | | |
| 329 | EWS | NP_005234.1 | Transcriptional | Y278 | QDHPSSMGVyGQESGGFSGPGENR | SEQ ID NO: 330 |
| | | | regulator | | | |
| 330 | EWS | NP_005234.1 | Transcriptional | Y417 | GDATVSyEDPPTAK | SEQ ID NO: 331 |
| | | | regulator | | | |
| 331 | FBP1 | NP_003893.2 | Transcriptional | Y626 | QQAAYyAQTSPQGMPQHPPAPQGQ | SEQ ID NO: 332 |
| | | | regulator | | | |
| 332 | FBP3 | NP_003925.1 | Transcriptional | Y51 | IDSIPHLNNSTPLVDPSVyGYGVQK | SEQ ID NO: 333 |
| | | | regulator | | | |
| 333 | FLI1 | NP_002008.2 | Transcnptional | Y263 | NTEQRPQPDPyQILGPTSSR | SEQ ID NO. 334 |
| | | | regulator | | | |
| 334 | FLI1 | NP_002008.2 | Transcriptional | Y332 | SKPNMNyDKLSR | SEQ ID NO: 335 |
| | | | regulator | | | |
| 335 | CDA02 | NP_114414.2 | Translational | Y258 | TGASYYGEQTLHyIATNGESAVVQLPK | SEQ ID NO: 336 |
| | | | regulator | | | |
| 336 | DDX48 | NP_055555.1 | Translational | Y54 | GIyAYGFEKPSAIQQR | SEQ ID NO: 337 |
| | | | regulator | | | |
| 337 | eEF-2 | NP_001952.1 | Translational | Y443 | IMGPNYTPGKKEDLyLKPIQR | SEQ ID NO: 338 |
| | | | regulator | | | |
| 338 | eEF-2 | NP_001952.1 | Translational | Y760 | LMEPIYLVEIQCPEQVVGGIyGVLNR | SEQ ID NO: 339 |
| | | | regulator | | | |
| 339 | eEF-2 | NP_001952.1 | Translational | Y79 | STAISLFyELSENDLNFIKQSK | SEQ ID NO: 340 |
| | | | regulator | | | |
| 340 | eIF2A | NP_004085.1 | Translational | Y82 | VDKEKGyIDLSK | SEQ ID NO: 341 |
| | | | regulator | | | |
| 341 | eIF2B | NP_003899.2 | Translational | Y298 | LyFLQCETCHSR | SEQ ID NO: 342 |
| | | | regulator | | | |
| 342 | eIF2B- | NP_001405.1 | Translational | Y130 | DGATILTHAySR | SEQ ID NO: 343 |
| | alpha | | regulator | | | |
| 343 | eIF2S3 | NP_001406.1 | Translational | Y238 | YNIEVVCEyIVK | SEQ ID NO: 344 |
| | | | regulator | | | |
| 344 | eIF3- | NP_003748.1 | Translational | Y241 | TERPVNSAALSPNYDHVVLGGGQEAMD | SEQ ID NO: 345 |
| | beta | | regulator | | VTTTSTR | |
| 345 | eIF3- | NP_003745.1 | Translational | Y241 | YAyYDTER | SEQ ID NO: 346 |
| | epsilon | | regulator | | | |
| 346 | eIF3-eta | NP_003742.2 | Translational | Y449 | MTLDTLSIyETPSMGLLDKK | SEQ ID NO: 347 |
| | | | regulator | | | |
| 347 | eIF3S6IP | NP_057175.1 | Translational | Y72 | TVSDLIDQKVyELQASR | SEQ ID NO: 348 |
| | | | regulator | | | |
| 348 | eIF3S6IP | NP_057175.1 | Translational | Y89 | VSSDVIDQKVyEIQDIYENSWTK | SEQ ID NO: 349 |
| | | | regulator | | | |
| 349 | eIF3- | NP_003741.1 | Translational | Y32 | KQPALDVLyDVMK | SEQ ID NO: 350 |
| | theta | | regulator | | | |
| 350 | eIF3- | NP_003741.1 | Translational | Y382 | FNVLQyVVPEVK | SEQ ID NO: 351 |
| | theta | | regulator | | | |
| 351 | eIF3- | NP_003744.1 | Translational | Y30 | DMPyQPFSKGDR | SEQ ID NO: 352 |
| | zeta | | regulator | | | |
| 352 | eIF4A1 | NP_001407.1 | Translational | Y48 | GIyAYGFEKPSAIQQR | SEQ ID NO: 353 |
| | | | regulator | | | |
| 353 | eIF4A1 | NP_001407.1 | Translational | Y70 | GyDVIAQAQSGTGK | SEQ ID NO: 354 |
| | | | regulator | | | |
| 354 | eIF4B | NP_001408.2 | Translational | Y141 | LKGFGyAEFEDLDSLLSALSLNEESLG | SEQ ID NO: 355 |
| | | | regulator | | NRR | |
| 355 | eIF4B | NP_001408.2 | Translational | Y285 | AFGSGyR | SEQ ID NO: 356 |
| | | | regulator | | | |
| 356 | eIF4B | NP_001408.2 | Translational | Y63 | WHSNDDDVyRAPPIDRSILPT | SEQ ID NO: 357 |
| | | | regulator | | | |
| 357 | eIE5A | NP_001961.1 | Translational | Y98 | RNDFQLIGIQDGyLSLLQDSGEVR | SEQ ID NO: 358 |
| | | | regulator | | | |
| 358 | AIM2 | NP_004824.1 | Tumor | Y74 | LNyMLLAKRLQEEKEKVDKQYK | SEQ ID NO: 359 |
| | | | suppressor | | | |
| 359 | AIM2 | NP_004824.1 | Tumor | Y92 | LNYMLLAKRLQEEKEKVDKQyK | SEQ ID NO: 360 |
| | | | suppressor | | | |
| 360 | apollon | NP_057336.2 | Ubiquitin | Y4461 | TAEIVyAATTSLR | SEQ ID NO: 361 |
| | | | conjugating | | | |
| | | | system | | | |
| 361 | ATG5 | NP_004840.1 | Ubiguitin | Y35 | IPTCFTLYQDEITEREAEPyYLLLPR | SEQ ID NO: 362 |
| | | | conjugating | | | |
| | | | system | | | |
| 362 | FAF-X | NP_001034679.2 | Ubiquitin | Y2439 | RPYTGNPQYTyNNWSPPVQSNETSNGY | SEQ ID NO: 363 |
| | | | conjugating | | FLER | |
| | | | system | | | |
| 363 | A2LP | NP_059867.2 | Unknown function | Y118 | GQSTGKGPPQSPVFEGVyNNSR | SEQ ID NO: 364 |
| 364 | A2LP | NP_059867.2 | Unknown function | Y145 | NGTTyEGIFK | SEQ ID NO: 365 |
| 365 | A2LP | NP_059867.2 | Unknown function | Y270 | TTyDSSLSSYTVPLEKDNSEEFR | SEQ ID NO: 366 |
| 366 | A2LP | NP_059867.2 | Unknown function | Y277 | TTYDSSLSSyTVPLEKDNSEEFR | SEQ ID NO: 367 |
| 367 | ACBD6 | NP_115736.1 | Unknown function | Y69 | EQLLYLyAR | SEQ ID NO: 368 |
| 368 | AF15q14 | NP_653091.2 | Unknown function | Y103 | KLEDNyCEITGMNTLLSAPIHTQMQQK | SEQ ID NO: 369 |
| 369 | AF15q14 | NP_653091.2 | Unknown function | Y1438 | VYVDDIyVIPQPHFSTDQPPLPK | SEQ ID NO: 370 |
| 370 | AF15q14 | NP_653091.2 | Unknown function | Y417 | ILAMTPESIySNPSIQGCK | SEQ ID NO: 371 |
| 371 | AF15q14 | NP_653091.2 | Unknown function | Y463 | MyCNPDAMSSLTEK | SEQ ID NO: 372 |
| 372 | ALKBH3 | NP_631917.1 | Unknown function | Y127 | TGIREDITyQQPR | SEQ ID NO: 373 |
| 373 | ANKRD13 | NP_149112.1 | Unknown function | Y520 | SQELSGPASNGGISQTNTyDAQYER | SEQ ID NO: 374 |
| 374 | ANKRD13 | NP_149112.1 | Unknown function | Y524 | SQELSGPASNGGISQTNTYDAQyER | SEQ ID NO: 375 |
| 375 | ANKRD13 | NP_149112.1 | Unknown function | Y91 | QGWTVLHEAVSTGDPEMVyTVLQHR | SEQ ID NO: 376 |
| 376 | ANKRD26 | NP_055730.1 | Unknown function | Y296 | NLEATyGTVR | SEQ ID NO: 377 |
| 377 | ANKS1 | NP_056060.2 | Unknown function | Y834 | IIASLADRPyEEPPQKPPR | SEQ ID NO: 378 |
| 378 | ANKS1 | NP_056060.2 | Unknown function | Y922 | LTLRPPSLAAPyAPVQSWQHQPEK | SEQ ID NO: 379 |
| 379 | ARID3B | NP_006456.1 | Unknown function | Y295 | TQyMKYLYAYECEKKALSSPAELQAAI | SEQ ID NO: 380 |
| | | | | | DGNRR | |
| 380 | ARID3B | NP_006456.1 | Unknown function | Y298 | TQYMKyLYAYECEKKALSSPAELQAAI | SEQ ID NO: 381 |
| | | | | | DGNRR | |
| 381 | ARID3B | NP_006456.1 | Unknown function | Y300 | TQYMKYLyAYECEKKALSSPAELQAAI | SEQ ID NO: 382 |
| | | | | | DGNRR | |
| 382 | ARID3B | NP_006456.1 | Unknown function | Y302 | TQYMKYLYAyECEKKALSSPAELQAAI | SEQ ID NO: 383 |
| | | | | | DGNRR | |
| 383 | ARMET | NP_006001.2 | Unknown function | Y79 | LCyYIGATDDAATK | SEQ ID NO: 384 |
| 384 | ARS2 | NP_056992.4 | Unknown function | Y630 | IVHSLDYYNTCEyPNEDEMPNR | SEQ ID NO: 385 |
| 385 | ataxin-2 | NP_002964.2 | Unknown function | Y471 | VALENDDRSEEEKyTAVQR | SEQ ID NO: 386 |
| 386 | ataxin-3 | NP_004984.2 | Unknown function | Y58 | MAEGGVTSEDyR | SEQ ID NO: 387 |
| 387 | ATG9A | NP_076990.4 | Unknown function | Y209 | ELTELDIyHR | SEQ ID NO: 388 |
| 388 | ATP11C | NP_775965.2 | Unknown function | Y38 | TVFVGNHPVSETEAyIAQR | SEQ ID NO: 389 |
| 389 | BAT2 | NP_004629.2 | Unknown function | Y1003 | RDYSyER | SEQ ID NO: 390 |
| 390 | BAT2 | NP_004629.2 | Unknown function | Y2097 | VDLyQQASPPDALR | SEQ ID NO: 391 |
| 391 | BAT2 | NP_004629.2 | Unknown function | Y1082 | SEGSEyEEIPKR | SEQ ID NO: 392 |
| | iso2 | | | | | |
| 392 | BAT2D1 | NP_055987.2 | Unknown function | Y164 | EKETNDDNyGPGPSLRPPNVACWR | SEQ ID NO: 393 |
| 393 | BAT2D1 | NP_055987.2 | Unknown function | Y1971 | LPSAQTPNGTDyVASGK | SEQ ID NO: 394 |
| 394 | BAT2D1 | NP_055987.2 | Unknown function | Y241 | LNGQQAALASQyR | SEQ ID NO: 395 |
| 395 | BAT2D1 | NP_055987.2 | Unknown function | Y253 | AMMPPYMFQQyPR | SEQ ID NO: 396 |
| 396 | BAT2D1 | NP_055987.2 | Unknown function | Y663 | PAVLSGyFK | SEQ ID NO: 397 |
| 397 | BCDIN3 | NP_062552.2 | Unknown function | Y418 | KFQYGNyCK | SEQ ID NO: 398 |
| 398 | BK1048 | NP_065170.1 | Unknown function | Y101 | LyHNLQEYAK | SEQ ID NO: 399 |
| | E9.6 | | | | | |
| 399 | BK1048 | NP_065170.1 | Unknown function | Y107 | LYHNLQEyAK | SEQ ID NO: 400 |
| | E9.6 | | | | | |
| 400 | BMS1L | NP_055568.2 | Unknown function | Y283 | KVSLyGYLR | SEQ ID NO: 401 |
| 401 | BRWD2 | NP_060587.8 | Unknown function | Y1086 | YLQTyGEWNR | SEQ ID NO: 402 |
| 402 | BTLA | AAP44003.1 | Unknown function | Y257 | MQEGSEVCSNPCLEENKPGIVyASLNH | SEQ ID NO: 403 |
| | | | | | SVIGLNSR | |
| 403 | BTLA | NP_861445.3 | Unknown function | Y282 | NVKEAPTEyASICVR | SEQ ID NO: 404 |
| 404 | BXDC2 | NP_060791.3 | Unknown function | Y127 | KKQDLyMWLSNSPHGPSAK | SEQ ID NO: 405 |
| 405 | C11orf2 | NP_037397.2 | Unknown function | Y579 | LLTHyVKVQGLVISQMLRKSVETR | SEQ ID NO: 406 |
| 406 | C13orf10 | NP_071401.3 | Unknown function | Y602 | FIKVyWHR | SEQ ID NO: 407 |
| 407 | C14orf112 | NP_057552.1 | Unknown function | Y67 | LKENKISLESEyEKIK | SEQ ID NO: 408 |
| 408 | C14orf130 | NP_060578.2 | Unknown function | Y271 | RVDGMQyYCS | SEQ ID NO: 409 |
| 409 | C14orf130 | NP_060578.2 | Unknown function | Y272 | RVDGMQYyCS | SEQ ID NO: 410 |
| 410 | C14orf172 | NP_689520.2 | Unknown function | Y67 | GGWVyVLHPTPELWTLNLPHR | SEQ ID NO: 411 |
| 411 | C1orf123 | NP_060357.1 | Unknown function | Y45 | CGNCGEISDKWQyIR | SEQ ID NO: 412 |
| 412 | C1orf73 | NP_056249.1 | Unknown function | Y935 | SLEDPySQQIR | SEQ ID NO: 413 |
| 413 | C1QDC1 | NP_076414.2 | Unknown function | Y146 | HKIRNIEKKKLKLEDyKDR | SEQ ID NO: 414 |
| 414 | C1QDC1 | NP_076414.2 | Unknown function | Y849 | GLPSISNGNySQLQFQAR | SEQ ID NO: 415 |
| 415 | C20orf27 | NP_001034229.1 | Unknown function | Y188 | CVGAELEyDSEHSDWHGFD | SEQ ID NO: 416 |
| 416 | C20orf72 | NP_443097.1 | Unknown function | Y342 | NQNIQKPEySE | SEQ ID NO: 417 |
| 417 | C21orf5 | NP_005119.2 | Unknown function | Y1330 | SYYPCyLKVSHR | SEQ ID NO: 418 |
| 418 | C2F | NP_006322.3 | Unknown function | Y222 | TEKMVSISNyPLSAAL | SEQ ID NO: 419 |
| 419 | C2F | NP_006322.3 | Unknown function | Y61 | VGKTyELLNCDKHK | SEQ ID NO: 420 |
| 420 | C3orf6 | NP_777568.1 | Unknown function | Y146 | AYADSYYyEDGGMKPR | SEQ ID NO: 421 |
| | iso2 | | | | | |
| 421 | C6orf60 | NP_078857.4 | Unknown function | Y99 | MKQQALTEFEAyKHR | SEQ ID NO: 422 |
| 422 | C7orf28 | NP_056437.4 | Unknown function | Y437 | AMSDyWVVGKK | SEQ ID NO: 423 |
| | A | | | | | |
| 423 | C9orf10 | NP_055427.2 | Unknown function | Y418 | NLTEQNSySNIPHEGK | SEQ ID NO: 424 |
| 424 | C9orf140 | NP_848543.1 | Unknown function | Y76 | QVAPASGyLTFER | SEQ ID NO: 425 |
| 425 | CALCO | NP_005822.1 | Unknown function | Y204 | VKEQKDyWETELLQLK | SEQ ID NO: 426 |
| | CO2 | | | | | |
| 426 | CAMSA | NP_056262.1 | Unknown function | Y1259 | CYYPDTEEIyKL | SEQ ID NO: 427 |
| | P1 | | | | | |
| 427 | CANP | NP_945185.1 | Unknown function | Y348 | IRNyYFCSLPR | SEQ ID NO: 428 |
| 428 | CANP | NP_945185.1 | Unknown function | Y349 | IRNYyFCSLPR | SEQ ID NO: 429 |
| 429 | CAP1 | NP_006358.1 | Unknown function | Y164 | EMNDAAMFyTNR | SEQ ID NO: 430 |
| 430 | CAP1 | NP_006358.1 | Unknown function | Y354 | AYIyKCVNT | SEQ ID NO: 431 |
| 431 | CapZIP | NP_443094.2 | Unknown function | Y156 | SRPSEAEEVPVSFDQPPEGSHLPCyNK | SEQ ID NO: 432 |
| 432 | CASKIN2 | NP_065804.1 | Unknown function | Y336 | IGyFPPGIVEVVSKR | SEQ ID NO: 433 |
| 433 | CCDC59 | NP_054886.1 | Unknown function | Y176 | AQEEyEQIQAK | SEQ ID NO: 434 |
| 434 | CCDC64 | NP_997194.2 | Unknown function | Y131 | QyEQMHKELTDKLEHLEQEK | SEQ ID NO: 435 |
| 435 | CCDC8 | NP_114429.1 | Unknown function | Y197 | WREyVSQVSWGKLKRR | SEQ ID NO: 436 |
| 436 | CDV-3 | NP_060018.1 | Unknown function | Y169 | TAPVQAPPAPVIVTETPEPAMTSGVyR | SEQ ID NO: 437 |
| | | | | | PPGAR | |
| 437 | CDV-3 | NP_060018.1 | Unknown function | Y257 | LQLDNQYAVLENQKSSHSQyN | SEQ ID NO: 438 |
| 438 | CHCHD2 | NP_057223.1 | Unknown function | Y99 | SGGSNAEPARPDITyQEPQGTQPAQQQ | SEQ ID NO: 439 |
| | | | | | QPCLY | |
| 439 | CHD-6 | NP_15597.3 | Unknown function | Y1540 | AARTLyRIELLR | SEQ ID NO: 440 |
| 440 | CKAP2L | NP_689728.2 | Unknown function | Y113 | RLTSECVSSNPySKPSSK | SEQ ID NO: 441 |
| 441 | CKAP2L | NP_689728.2 | Unknown function | Y310 | SQyERPNETK | SEQ ID NO: 442 |
| 442 | COPG2 | NP_036265.3 | Unknown function | Y826 | NSHSLyLAGIFR | SEQ ID NO: 443 |
| 443 | CRMP-2 | NP_001377.1 | Unknown function | Y32 | IVNDDQSFyADIYMEDGLIK | SEQ ID NO: 444 |
| 444 | CRMP-2 | NP_001377.1 | Unknown function | Y431 | THNSSLEyNIFEGMECR | SEQ ID NO: 445 |
| 445 | CRMP-2 | NP_001377.1 | Unknown function | Y499 | GLyDGPVCEVSVTPK | SEQ ID NO: 446 |
| 446 | CWF19L2 | NP_689647.1 | Unknown function | Y479 | FMGKTDGDyYTLDDMFVSK | SEQ ID NO: 447 |
| 447 | DCBLD1 | NP_775945.1 | Unknown function | Y506 | QIKyPFAR | SEQ ID NO: 448 |
| 448 | DDX29 | NP_061903.2 | Unknown function | Y811 | YQEyIPVQTGAHADLNPFYQK | SEQ ID NO: 449 |
| 449 | DDX48 | NP_055555.1 | Unknown function | Y202 | MLVLDEADEMLNKGFKEQIyDVYR | SEQ ID NO: 450 |
| 450 | DDX6 | NP_004388.1 | Unknown function | Y301 | GVTQyYAYVTER | SEQ ID NO: 451 |
| 451 | DDX6 | NP_004388.1 | Unknown function | Y429 | NLITyDDRFNLKSIEEQLGTEIKPIPS | SEQ ID NO: 452 |
| 452 | DHX30 | NP_055781.2 | Unknown function | Y929 | ENYLEENLLyAPSLR | SEQ ID NO: 453 |
| 453 | DHX57 | NP_945314.1 | Unknown function | Y1261 | NDGyVHIHPSSVNYQVR | SEQ ID NO: 454 |
| 454 | DHX57 | NP_945314.1 | Unknown function | Y1271 | NDGYVHIHPSSVNyQVR | SEQ ID NO: 455 |
| 455 | DJ-1 | NP_009193.2 | Unknown function | Y139 | MMNGGHyTYSENRVEK | SEQ ID NO: 456 |
| 456 | DKFZP4 | NP_056268.1 | Unknown function | Y187 | TSHALDSHILDyYENPAIKEDVSTL | SEQ ID NO: 457 |
| | 34F091 | | | | | |
| 457 | DKFZP4 | NP_056268.1 | Unknown function | Y188 | TSHALDSHILDYyENPAIKEDVSTL | SEQ ID NO: 458 |
| | 34F091 | | | | | |
| 458 | DKFZp5 | NP_061039.2 | Unknown function | Y110 | LEEEALyAAQR | SEQ ID NO: 459 |
| | 64C182 | | | | | |
| 459 | DKFZp5 | NP_061039.2 | Unknown function | Y137 | IVQQyHPSNNGEYQSSGPEDDFESCLR | SEQ ID NO: 460 |
| | 64C182 | | | | | |
| 460 | DKFZp5 | NP_061039.2 | Unknown function | Y145 | IVQQYHPSNNGEyQSSGPEDDFESCLR | SEQ ID NO: 461 |
| | 640182 | | | | | |
| 461 | DKFZp6 | NP_689894.1 | Unknown function | Y209 | NKAKPEPDILEEEKIyAYPSNITSETG | SEQ ID NO: 462 |
| | 86K0275 | | | | F | |
| 462 | DKFZp6 | NP_689894.1 | Unknown function | Y211 | AKPEPDILEEEKIYAyPSNITSETGFR | SEQ ID NO: 463 |
| | 86K0275 | | | | | |
| 463 | DKFZp7 | NP_001073027.1 | Unknown function | Y222 | AYYEFREEAyHSR | SEQ ID NO: 464 |
| | 62N1910 | | | | | |
| 464 | DKFZp7 | NP_001073027.1 | Unknown function | Y740 | YYRNyYGYQGYR | SEQ ID NO: 465 |
| | 62N1910 | | | | | |
| 465 | DNA2L | XP_943722.1 | Unknown function | Y1029 | AGCSPSDIGIIAPyRQQLKIINDLLAR | SEQ ID NO: 466 |
| 466 | DOCK11 | NP_653259.3 | Unknown function | Y1188 | LAGRDTLySCAAMPNSASRDEFPCGFT | SEQ ID NO: 467 |
| | | | | | SPANR | |
| 467 | DOCK11 | NP_653259.3 | Unknown function | Y1223 | GSLSTDKDTAyGSFQNGHGIK | SEQ ID NO: 468 |
| 468 | DOCK11 | NP_653259.3 | Unknown function | Y57 | KTQIySDPLR | SEQ ID NO: 469 |
| 469 | DOCK8 | NP_982272.1 | Unknown function | Y547 | SSGPEFLQEVyTAVTYHNK | SEQ ID NO: 470 |
| 470 | DRPLA | NP_001931.2 | Unknown function | Y905 | NHPFyVPLGAVDPGLLGYNVPALYSSD | SEQ ID NO: 471 |
| | | | | | PAAR | |
| 471 | DVL2 | NP_004413.1 | Unknown function | Y362 | CWDPSPQAyFTLPR | SEQ ID NO: 472 |
| 472 | ELYS | NP_056261.3 | Unknown function | Y1790 | EISEASENIySDVR | SEQ ID NO: 473 |
| 473 | ETEA | NP_055428.1 | Unknown function | Y81 | IYSyVVSRPQPR | SEQ ID NO: 474 |
| 474 | FAM20B | NP_055679.1 | Unknown function | Y138 | SRDHWEGEPyAGY | SEQ ID NO: 475 |
| 475 | FAM29A | NP_060115.3 | Unknown function | Y418 | SFDPASEEVyAK | SEQ ID NO: 476 |
| 476 | FAM50B | NP_036267.1 | Unknown function | Y53 | FSAHyDAVEAELK | SEQ ID NO: 477 |
| 477 | FAM83A | NP_116288.2 | Unknown function | Y138 | SSATVYFQTVK | SEQ ID NO: 478 |
| 478 | FAM83A | NP_116288.2 | Unknown function | Y398 | PHDGPPAAVySNLGAYRPTR | SEQ ID NO: 479 |
| 479 | FAM83D | NP_112181.2 | Unknown function | Y259 | TITGNIyYAR | SEQ ID NO: 480 |
| 480 | FAM98B | NP_775882.2 | Unknown function | Y147 | NSQLDKNSEVyQEVQAMFDTLGIPK | SEQ ID NO: 481 |
| 481 | EGER1O | NP_056448.1 | Unknown function | Y46 | RVEAMKQyQEEIQELNEVAR | SEQ ID NO: 482 |
| | P2 | | | | | |
| 482 | FIP1L1 | NP_112179.2 | Unknown function | Y175 | KPGADLSDyFNYGFNEDTWK | SEQ ID NO: 483 |
| 483 | FIP1L1 | NP_112179.2 | Unknown function | Y454 | AFPYGNVAFPHLPGSAPSWPSLVDTSK | SEQ ID NO: 484 |
| | | | | | QWDYyAR | |
| 484 | FLJ11305 | NP_060856.1 | Unknown functibn | Y286 | QAEEyLSFAFEHCHR | SEQ ID NO: 485 |
| 485 | FLJ11773 | NP_068753.2 | Unknown function | Y164 | HEyLPK | SEQ ID NO: 486 |
| 486 | adaptin, | NP_001273.1 | Vesicle protein | Y277 | DSDYyNMLLK | SEQ ID NO: 487 |
| | beta | | | | | |
| 487 | adaptin, | NP_001273.1 | Vesicle protein | Y874 | LQNNNVyTIAK | SEQ ID NO: 488 |
| | beta | | | | | |
| 488 | adaptin, | NP_001273.1 | Vesicle protein | Y928 | CRAPEVSQYIyQVYDSILKN | SEQ ID NO: 489 |
| | beta | | | | | |
| 489 | BET1 | NP_005859.1 | Vesicle protein | Y18 | AGLGEGVPPGNYGNyGYANSGYSACEE | SEQ ID NO: 490 |
| | | | | | ENERLTESLR | |
| 490 | CLH-17 | NP_004850.1 | Vesicle protein | Y1096 | AyEFAER | SEQ ID NO: 491 |
| 491 | CLH-17 | NP_004850.1 | Vesicle protein | Y1206 | LAELEEFINGPNNAHIQQVGDRCyDEK | SEQ ID NO: 492 |
| 492 | CLH-17 | NP_004850.1 | Vesicle protein | Y1211 | CYDEKMyDAAK | SEQ ID NO: 493 |
| 493 | COP, | NP_004757.1 | Vesicle protein | Y101 | VHMFEAHSDyIR | SEQ ID NO: 494 |
| | beta | | | | | |
| | prime | | | | | |
| 494 | COPE | NP_009194.2 | Vesicle protein | Y202 | LQDAyYIFQEMADK | SEQ ID NO: 495 |
| 495 | DYN2 | NP_004936.2 | Vesicle protein | Y125 | VySPHVLNLTLIDLPGITK | SEQ ID NO: 496 |
| 496 | EHD1 | NP_006786.2 | Vesicle protein | Y29 | EPELFQTVAEGLRQLyAQK | SEQ ID NO: 497 |
| 497 | EHD1 | NP_006786.2 | Vesicle rotein | Y448 | DKPTyDEIFYTLSPVNGK | SEQ ID NO: 498 |
| 498 | epsin 1 | NP_037465.2 | Vesicle protein | Y17 | NIVHNySEAEIK | SEQ ID NO: 499 |

The short name for each protein in which a phosphorylation site has presently been identified is provided in Column A, and its SwissProt accession number (human) is provided Column B. The protein type/group into which each protein falls is provided in Column C. The identified tyrosine residue at which phosphorylation occurs in a given protein is identified in Column D, and the amino acid sequence of the phosphorylation site encompassing the tyrosine residue is provided in Column E (lower case y=the tyrosine (identified in Column D)) at which phosphorylation occurs. Table 1 above is identical to FIG. 2, except that the latter includes the disease and cell type(s) in which the particular phosphorylation site was identified (Columns F and G).

One of skill in the art will appreciate that, in many instances the utility of the instant invention is best understood in conjunction with an appreciation of the many biological roles and significance of the various target signaling proteins/polypeptides of the invention. The foregoing is illustrated in the following paragraphs summarizing the knowledge in the art relevant to a few non-limiting representative peptides containing selected phosphorylation sites according to the invention.

ADA (P00813), phosphorylated at Y28, Y66, Y307, Y347, is among the proteins listed in this patent. ADA, Adenosine deaminase, plays a role in immune response, binds to CD26 (DPP4), altered activity or expression is associated with various cancers and autoimmune diseases; gene polymorphism is associated with autism. This protein has potential diagnostic and/or therapeutic implications based on the following findings. Abnormal mRNA splicing of ADA causes severe combined immunodeficiency (Hum Mol Genet 4: 2081-7 (1995)). Decreased expression of ADA protein correlates with increased response to drug associated with stomach ulcer (Eur J Pharmacol 205: 101-3 (1991)). Polymorphism in the ADA gene correlates with early onset form of type II diabetes mellitus (Proc Natl Acad Sci USA 88: 1484-8 (1991)). Splice site mutation in the ADA gene causes severe combined immunodeficiency (Am J Hum Genet 55: 59-68(1994)). Mutation in the ADA gene causes autosomal recessive form of severe combined immunodeficiency (J Immunol 166: 1698-702 (2001)). Increased expression of ADA protein correlates with peritoneal tuberculosis (Gut 36: 419-21 (1995)). Polymorphism in the ADA gene correlates with abnormal response to nutrient associated with type II diabetes mellitus (Proc Natl Acad Sci USA 88: 1484-8 (1991)). Absence of the adenosine deaminase activity of ADA causes severe combined immunodeficiency (Am J Hum Genet 63: 1049-59 (1998)). Deletion mutation in the ADA gene causes immunologic deficiency syndromes (Genomics 7: 486-90 (1990)). Nonsense mutation in the ADA gene causes severe combined immunodeficiency (Hum Mol Genet 4: 2081-7 (1995)). Decreased expression of ADA protein correlates with increased response to drug associated with stomach ulcer (Eur J Pharmacol 243: 301-3 (1993)). Absence of the adenosine deaminase activity of ADA causes late onset form of severe combined immunodeficiency (Am J Hum Genet 63: 1049-59 (1998)). Loss of function mutation in the ADA gene causes decreased immune system function associated with immunologic deficiency syndromes (J Immunol 153: 2331-9 (1994)). Increased expression of ADA in blood correlates with autoimmune thyroiditis (J Cell Biochem 89: 550-5 (2003)). Absence of the adenosine deaminase activity of ADA correlates with severe combined immunodeficiency (Science 296: 2410-3 (2002)). Loss of function mutation in the ADA gene causes late onset form of immunologic deficiency syndromes (J Immunol 153: 2331-9 (1994)). Splice site mutation in the ADA gene correlates with late onset form of severe combined immunodeficiency (J Clin Invest 92: 2291-302 (1993)). Decreased adenosine deaminase activity of ADA causes late onset form of immunologic deficiency syndromes (J Immunol 153: 2331-9 (1994)). Deletion mutation in the ADA gene correlates with late onset form of severe combined immunodeficiency (J Clin Invest 92: 2291-302 (1993)). Missense mutation in the ADA gene correlates with late onset form of severe combined immunodeficiency (J Clin Invest 92: 2291-302 (1993)). Decreased adenosine deaminase activity of ADA causes decreased immune system function associated with immunologic deficiency syndromes (J Immunol 153: 2331-9 (1994)). ADA map position may correlate with disease susceptibility associated with type II diabetes mellitus (Hum Mol Genet 6: 1401-8 (1997)). Absence of the adenosine deaminase activity of ADA causes severe combined immunodeficiency (J Biol Chem 273: 5093-100 (1998)). Polymorphism in the ADA gene correlates with abnormal response to nutrient associated with type II diabetes mellitus (PNAS 88: 1484-8 (1991)). Deletion mutation in the ADA gene causes severe combined immunodeficiency (J Immunol 149: 3107-12 (1992)). Polymorphism in the ADA gene correlates with early onset form of type II diabetes mellitus (Proc Natl Acad Sci USA 88: 1484-8 (1991)). Absence of the adenosine deaminase activity of ADA causes autosomal recessive form of severe combined immunodeficiency (J Immunol 166: 1698-702 (2001)). Polymorphism in the ADA gene correlates with early onset form of type II diabetes mellitus (PNAS 88: 1484-8 (1991)). Abnormal expression of ADA protein correlates with non-Hodgkin's lymphoma (Cancer 70: 20-7 (1992)). Absence of the adenosine deaminase activity of ADA causes lymphopenia (Am J Hum Genet 63: 1049-59 (1998)). Abnormal enhancer splicing of ADA correlates with early onset form of severe combined immunodeficiency (Hum Mol Genet 4: 2081-7 (1995)). Increased expression of ADA in blood correlates with Graves' disease (J Cell Biochem 89: 550-5 (2003)). Missense mutation in the ADA gene causes severe combined immunodeficiency (Hum Mol Genet 6: 2271-8 (1997)). Absence of the adenosine deaminase activity of ADA causes severe combined immunodeficiency (JBC 273: 5093-100 (1998)). Increased expression of ADA protein correlates with Graves' disease (Endocr Res 28: 207-15 (2002)). ADA map position correlates with obesity (J Clin Invest 100: 1240-7 (1997)). Increased expression of ADA protein correlates with autoimmune thyroiditis (Endocr Res 28: 207-15 (2002)). Increased expression of ADA protein correlates with more severe form of stomach neoplasms (Cancer Lett 109: 199-202 (1996)). Decreased expression of ADA protein correlates with inborn errors of purine-pyrimidine metabolism (J Clin Invest 103: 833-41 (1999)). Polymorphism in the ADA gene correlates with abnormal response to nutrient associated with type II diabetes mellitus (Proc Natl Acad Sci USA 88: 1484-8 (1991)). (PhosphoSite®, Cell Signaling Technology (Danvers, Mass.), Human PSD™, Biobase Corporation, (Beverly, Mass.)).

Afadin (P55196), phosphorylated at Y568, Y1675, is among the proteins listed in this patent. Afadin, Mixed lineage-leukemia translocation to 4 homolog (afadin), intercellular junction protein, negatively regulates cell adhesion, may regulate actin polymerization; MLLT4-ALL-1 (MLL) fusion variant is associated with acute myeloid leukemia. This protein has potential diagnostic and/or therapeutic implications based on the following findings. MLLT4 map position may correlate with carcinoma tumors associated with ovarian neoplasms (Cancer Res 56: 5586-9 (1996)). Translocation of the MLLT4 gene correlates with acute myelocytic leukemia (Cancer Res 53: 5624-8 (1993)). Translocation of the MLLT4 gene correlates with acute monocytic leukemia (Blood 87: 2496-505 (1996)). Translocation of the MLLT4 gene correlates with acute myelocytic leukemia (Blood 87: 2496-505 (1996)). (PhosphoSite®, Cell Signaling Technology (Danvers, Mass.), Human PSD™, Biobase Corporation, (Beverly, Mass.)).

ANXA11 (P50995), phosphorylated at Y365, is among the proteins listed in this patent. ANXA11, Annexin A11 (annexin XI), member of the annexin family of calcium-dependent phospholipid-binding proteins, binds ALG-2 (PDCD6), may play roles in phagocytosis and mitosis; autoantibodies are detected in sera of patients with autoimmune disease. This protein has potential diagnostic and/or therapeutic implications based on the following findings. (PhosphoSite®, Cell Signaling Technology (Danvers, Mass.), Human PSD™, Biobase Corporation, (Beverly, Mass.)).

ANXA2 (P07355), phosphorylated at Y198, is among the proteins listed in this patent. ANXA2, Annexin A2, plasmin reductase and tissue-type plasminogen activator (PLAT) receptor, regulates plasmin activity and cell migration, marker for various cancers (prostate, brain, breast, lung, pancreas, colorectal) and for heart failure. This protein has potential diagnostic and/or therapeutic implications based on the following findings. Increased expression of ANXA2 mRNA may correlate with drug-resistant form of colorectal neoplasms (Cancer Res 63: 4602-6 (2003)). Viral exploitation of the ANXA2 protein may correlate with cytomegalovirus infections (Biochemistry Usa 38: 5089-95 (1999)). Abnormal nucleus localization of ANXA2 correlates with astrocytoma (Oncol Res 6: 561-7 (1994)). Increased presence of ANXA2 autoimmune antibody correlates with squamous cell carcinoma associated with lung neoplasms (PNAS 98: 9824-9 (2001)). Increased expression of ANXA2 protein correlates with increased occurrence of death associated with colorectal neoplasms (Cancer 92: 1419-26 (2001)). Increased expression of ANXA2 mRNA correlates with glioblastoma tumors associated with brain neoplasms (Cancer Res 52: 6871-6 (1992)). Abnormal expression of ANXA2 mRNA may correlate with B-cell lymphoma (Biochim Biophys Acta 1313: 295-301 (1996)). Increased expression of ANXA2 protein correlates with increased severity of carcinoma associated with colorectal neoplasms (Cancer 92: 1419-26 (2001)). Decreased expression of ANXA2 mRNA correlates with esophageal neoplasms associated with squamous cell carcinoma (Int J Cancer 106: 327-33 (2003)). Increased presence of ANXA2 autoimmune antibody correlates with adenocarcinoma tumors associated with lung neoplasms (PNAS 98: 9824-9 (2001)). Increased expression of ANXA2 mRNA may correlate with increased response to drug associated with breast neoplasms (Cancer Res 63: 4602-6 (2003)). Increased presence of ANXA2 autoimmune antibody correlates with squamous cell carcinoma associated with lung neoplasms (Proc Natl Acad Sci USA 98: 9824-9 (2001)). Viral exploitation of the ANXA2 protein may correlate with cytomegalovirus infections (Biochemistry 38: 5089-95 (1999)). Increased presence of ANXA2 autoimmune antibody correlates with adenocarcinoma tumors associated with lung neoplasms (Proc Natl Acad Sci USA 98: 9824-9 (2001)). Increased presence of ANXA2 autoimmune antibody correlates with squamous cell carcinoma associated with lung neoplasms (Proc Natl Acad Sci USA 98: 9824-9 (2001)). Decreased expression of ANXA2 mRNA correlates with squamous cell carcinoma associated with esophageal neoplasms (Int J Cancer 106: 327-33 (2003)). Increased phosphorylation of ANXA2 may correlate with B-cell lymphoma (Biochim Biophys Acta 1313: 295-301 (1996)). Increased presence of ANXA2 autoimmune antibody correlates with adenocarcinoma tumors associated with lung neoplasms (Proc Natl Acad Sci USA 98: 9824-9 (2001)). Increased expression of ANXA2 protein may correlate with malignant form of colorectal neoplasms (Cancer 92: 1419-26 (2001)). Increased expression of ANXA2 protein correlates with glioblastoma tumors associated with brain neoplasms (Oncol Res 6: 561-7 (1994)). Decreased expression of ANXA2 protein may cause increased cell migration associated with prostatic neoplasms (Oncogene 22: 1475-85 (2003)). Increased expression of ANXA2 protein correlates with malignant form of pancreatic neoplasms (Carcinogenesis 14: 2575-9 (1993)). Increased expression of ANXA2 mRNA correlates with astrocytoma tumors associated with brain neoplasms (Cancer Res 52: 6871-6 (1992)). Increased expression of ANXA2 protein correlates with more severe form of glioblastoma (Oncol Res 6: 561-7 (1994)). Increased expression of ANXA2 protein correlates with pancreatic neoplasms (Oncogene 16: 625-33 (1998)). Increased expression of ANXA2 mRNA correlates with more severe form of astrocytoma (Cancer Res 52: 6871-6 (1992)). Increased expression of ANXA2 protein correlates with carcinoma tumors associated with colorectal neoplasms (Cancer 92: 1419-26 (2001)). Decreased expression of ANXA2 mRNA correlates with small cell carcinoma associated with lung neoplasms (Genomics 61: 5-14 (1999)). Increased expression of ANXA2 protein correlates with adenocarcinoma tumors associated with pancreatic neoplasms (Carcinogenesis 14: 2575-9 (1993)). Decreased expression of ANXA2 mRNA correlates with small cell carcinoma (Genomics 61: 5-14 (1999)). Decreased expression of ANXA2 mRNA correlates with prostatic neoplasms (Cancer Res 61: 6331-4 (2001)). (PhosphoSite®, Cell Signaling Technology (Danvers, Mass.), Human PSD™, Biobase Corporation, (Beverly, Mass.)).

ARRB2 (P32121), phosphorylated at Y48, is among the proteins listed in this patent. ARRB2, Arrestin beta 2, an adaptor that regulates GPCR desensitization by targeting GPCRs to clathrin-coated pits, abnormal thyroid expression correlates with thyroid nodules; mouse Arrb2 plays a role in the development of allergic asthma. This protein has potential diagnostic and/or therapeutic implications based on the following findings. Abnormal expression of ARRB2 in thyroid correlates with thyroid nodule (FEBS Lett 486: 208-212 (2000)). (PhosphoSite®, Cell Signaling Technology (Danvers, Mass.), Human PSD™, Biobase Corporation, (Beverly, Mass.)).

Ataxin-3 (P54252), phosphorylated at Y58, is among the proteins listed in this patent. ataxin-3, Ataxin 3, a ubiquitin protease that inhibits histone acetylation and may mediate ubiquitinated protein degradation; variants with an expanded polyglutamine region are associated with Machado-Joseph (spinocerebellar ataxia 3) and Parkinson disease. This protein has potential diagnostic and/or therapeutic implications based on the following findings. Mutation in the ATXN3 protein may cause abnormal protein folding associated with Machado-Joseph disease (Hum Mol Genet 8: 673-82 (1999)). Trinucleotide repeat instability in the ATXN3 gene may cause decreased myelination associated with Machado-Joseph disease (Hum Mol Genet 11: 1075-94 (2002)). Trinucleotide repeat instability in the ATXN3 gene may cause decreased peripheral nervous system function associated with Machado-Joseph disease (Hum Mol Genet 11: 1075-94 (2002)). Mutation in the ATXN3 gene causes increased incidence of familial form of Machado-Joseph disease (Am J Hum Genet 68: 523-8 (2001)). Trinucleotide repeat instability in the ATXN3 gene causes Machado-Joseph disease (Nat Genet 8: 221-8 (1994)). Trinucleotide repeat instability in the ATXN3 gene may cause decreased axonogenesis associated with Machado-Joseph disease (Hum Mol Genet 11: 1075-94 (2002)). Increased nuclear inclusion body localization of ATXN3 may cause Machado-Joseph disease (Neuron 19: 333-44 (1997)). Trinucleotide repeat instability in the ATXN3 gene may cause defective dentate gyrus development associated with Machado-Joseph disease (Hum Mol Genet 11: 1075-94 (2002)). Trinucleotide repeat instability in the ATXN3 protein may cause increased induction of apoptosis by intracellular signals associated with Machado-Joseph disease (Nat Genet 13: 196-202 (1996)). Trinucleotide repeat instability in the ATXN3 gene may cause decreased cerebellar cortex function associated with Machado-Joseph disease (Hum Mol Genet 11: 1075-94 (2002)). Trinucleotide repeat instability in the ATXN3 gene may cause defective cerebellum development associated with Machado-Joseph disease (Hum Mol Genet 11: 1075-94 (2002)). Trinucleotide repeat instability in the ATXN3 gene may cause defective pons development associated with Machado-Joseph disease (Hum Mol Genet 11: 1075-94 (2002)). Abnormal cleavage of ATXN3 may cause abnormal protein folding associated with Machado-Joseph disease (J Neurochem 89: 908-18 (2004)). (PhosphoSite®, Cell Signaling Technology (Danvers, Mass.), Human PSD™, Biobase Corporation, (Beverly, Mass.)).

ATM (Q13315), phosphorylated at Y1753, Y1763, Y2019, is among the proteins listed in this patent. ATM, Ataxia telangiectasia mutated, a serine/threonine kinase involved in apoptosis, DNA stability, cell cycle, and radiation response; gene mutation is associated with ataxia telangiectasia and implicated in B cell chronic lymphocytic leukemia. This protein has potential diagnostic and/or therapeutic implications based on the following findings. Mutation in the ATM gene may correlate with chronic lymphocytic leukemia (Blood 100: 603-9 (2002)). Loss of heterozygosity at the ATM gene correlates with chronic B-cell leukemia (Cancer Res 58: 4552-7 (1998)). Decreased expression of ATM protein correlates with decreased response to ionizing radiation associated with chronic B-cell leukemia (Blood 98: 814-22 (2001)). Abnormal mRNA splicing of ATM causes prolymphocytic leukemia (Blood 91: 3920-6 (1998)). Mutation in the ATM gene causes ataxia telangiectasia (Science 268: 1749-53 (1995)). Decreased expression of ATM protein correlates with increased occurrence of death associated with chronic B-cell leukemia (Cancer Res 58: 4552-7 (1998)). Splice site mutation in the ATM gene causes increased incidence of familial form of breast neoplasms (Cancer Res 63: 3325-33 (2003)). Nonsense mutation in the ATM gene causes prolymphocytic leukemia (Blood 91: 3920-6 (1998)). Point mutation in the ATM gene causes idiopathic form of mantle-cell lymphoma (PNAS 97: 2773-8 (2000)). Splice site mutation in the ATM gene causes increased incidence of non-familial form of breast neoplasms (Am J Hum Genet 66: 494-500 (2000)). Gain of function mutation in the ATM gene may cause increased incidence of familial form of breast neoplasms (J Natl Cancer Inst 94: 205-15 (2002)). Absence of the protein kinase activity of ATM may cause increased incidence of familial form of ovarian neoplasms (Cancer Res 63: 3325-33 (2003)). Missense mutation in the ATM gene correlates with decreased response to radiation associated with Hodgkin's disease (Blood 103: 283-90 (2004)). Mutation in the ATM gene causes ataxia telangiectasia (Am J Hum Genet 66: 494-500 (2000)). Decreased expression of ATM mRNA correlates with carcinoma tumors associated with breast neoplasms (Int J Cancer 78: 306-9 (1998)). Missense mutation in the ATM gene may cause increased incidence of non-familial form of breast neoplasms (Proc Natl Acad Sci USA 99: 925-30 (2002)). Frameshift mutation in the ATM gene correlates with T-cell lymphoma (Proc Natl Acad Sci USA 100: 5372-7 (2003)). Decreased protein kinase activity of ATM may cause increased incidence of familial form of breast neoplasms (PNAS 99: 925-30 (2002)). Missense mutation in the ATM gene correlates with increased incidence of familial form of breast neoplasms (Cancer 92: 479-87 (2001)). Decreased protein serine/threonine kinase activity of ATM causes decreased protein amino acid phosphorylation associated with acute lymphocytic leukemia (L1) (Blood 101: 3622-7 (2003)). Deletion mutation in the ATM gene may correlate with disease susceptibility associated with leukemia (Am J Hum Genet 62: 334-45 (1998)). Decreased expression of ATM mRNA may cause malignant form of breast neoplasms (Int J Cancer 78: 306-9 (1998)). Decreased protein kinase activity of ATM may cause increased incidence of familial form of breast neoplasms (Proc Natl Acad Sci USA 99: 925-30 (2002)). Missense mutation in the ATM gene may cause increased incidence of non-familial form of breast neoplasms (PNAS 99: 925-30 (2002)). Mutation in the ATM gene correlates with mantle-cell lymphoma (PNAS 100: 5372-7 (2003)). Nonsense mutation in the ATM gene causes idiopathic form of mantle-cell lymphoma (Proc Natl Acad Sci USA 97: 2773-8 (2000)). Missense mutation in the ATM gene may correlate with breast neoplasms (Proc Natl Acad Sci USA 99: 925-30 (2002)). Abnormal mRNA splicing of ATM causes idiopathic form of mantle-cell lymphoma (Proc Natl Acad Sci USA 97: 2773-8 (2000)). Decreased protein serine/threonine kinase activity of ATM correlates with increased occurrence of disease susceptibility associated with Hodgkin's disease (Blood 103: 283-90 (2004)). Abnormal mRNA splicing of ATM correlates with increased occurrence of disease susceptibility associated with Hodgkin's disease (Blood 103: 283-90 (2004)). Missense mutation in the ATM gene causes prolymphocytic leukemia (Blood 91: 3920-6 (1998)). Single nucleotide polymorphism in the ATM gene correlates with increased occurrence of disease susceptibility associated with Hodgkin's disease (Blood 103: 283-90 (2004)). Missense mutation in the ATM gene correlates with non-familial form of breast neoplasms (Am J Hum Genet 62: 334-45 (1998)). Nonsense mutation in the ATM gene causes idiopathic form of mantle-cell lymphoma (Proc Natl Acad Sci USA 97: 2773-8 (2000)). Deletion mutation in the ATM gene causes idiopathic form of mantle-cell lymphoma (Proc Natl Acad Sci USA 97: 2773-8 (2000)). Loss of heterozygosity at the ATM gene causes prolymphocytic leukemia (Blood 91: 3920-6 (1998)). Mutation in the ATM gene correlates with mantle-cell lymphoma (Proc Natl Acad Sci USA 100: 5372-7 (2003)). Deletion mutation in the ATM gene causes idiopathic form of mantle-cell lymphoma (Proc Natl Acad Sci USA 97: 2773-8 (2000)). Frameshift mutation in the ATM gene correlates with T-cell lymphoma (PNAS 100: 5372-7 (2003)). Deletion mutation in the ATM gene causes idiopathic form of mantle-cell lymphoma (PNAS 97: 2773-8 (2000)). Abnormal mRNA splicing of ATM causes idiopathic form of mantle-cell lymphoma (PNAS 97: 2773-8 (2000)). Absence of the protein kinase activity of ATM may cause increased incidence of familial form of breast neoplasms (Cancer Res 63: 3325-33 (2003)). Mutation in the ATM gene causes increased severity of necrosis associated with breast neoplasms (Br J Cancer 76: 1546-9 (1997)). Frameshift mutation in the ATM gene correlates with T-cell lymphoma (Proc Natl Acad Sci USA 100: 5372-7 (2003)). Deletion mutation in the ATM gene causes prolymphocytic leukemia (Blood 91: 3920-6 (1998)). Point mutation in the ATM gene causes idiopathic form of mantle-cell lymphoma (Proc Natl Acad Sci USA 97: 2773-8 (2000)). Loss of heterozygosity at the ATM locus correlates with breast neoplasms (Oncogene 14: 339-47 (1997)). Abnormal protein binding of ATM may cause increased incidence of familial form of ovarian neoplasms (Cancer Res 63: 3325-33 (2003)). Mutation in the ATM gene causes increased incidence of non-familial form of breast neoplasms (Am J Hum Genet 66: 494-500 (2000)). Decreased expression of ATM protein correlates with increased severity of disease progression associated with chronic B-cell leukemia (Cancer Res 58: 4552-7 (1998)). Missense mutation in the ATM gene causes diffuse large-cell lymphoma (Blood 100: 1430-7 (2002)). Missense mutation in the ATM gene causes increased incidence of familial form of ovarian neoplasms (Cancer Res 63: 3325-33 (2003)). Missense mutation in the ATM gene may correlate with lymphoma (Am J Hum Genet 62: 334-45 (1998)). Polymorphism in the ATM gene may correlate with abnormal response to radiation associated with breast neoplasms (Cancer Res 63: 8717-25 (2003)). Missense mutation in the Phosphatidylinositol 3- and 4-kinase domain of ATM causes decreased protein amino acid phosphorylation associated with acute lymphocytic leukemia (L1) (Blood 101: 3622-7 (2003)). Abnormal mRNA splicing of ATM causes diffuse large-cell lymphoma (Blood 100: 1430-7 (2002)). Abnormal mRNA splicing of ATM causes idiopathic form of mantle-cell lymphoma (Proc Natl Acad Sci USA 97: 2773-8 (2000)). Deletion mutation in the ATM gene may correlate with lymphoma (Am J Hum Genet 62: 334-45 (1998)). Missense mutation in the ATM gene correlates with increased incidence of early onset form of breast neoplasms (Cancer 92: 479-87 (2001)). Nonsense mutation in the ATM gene causes idiopathic form of mantle-cell lymphoma (PNAS 97: 2773-8 (2000)). Missense mutation in the ATM gene may correlate with breast neoplasms (Proc Natl Acad Sci USA 99: 925-30 (2002)). Decreased expression of ATM protein causes prolymphocytic leukemia (Blood 91: 3920-6 (1998)). Point mutation in the ATM gene causes idiopathic form of mantle-cell lymphoma (Proc Natl Acad Sci USA 97: 2773-8 (2000)). Missense mutation in the ATM gene may correlate with disease susceptibility associated with leukemia (Am J Hum Genet 62: 334-45 (1998)). Mutation in the ATM gene correlates with mantle-cell lymphoma (Proc Natl Acad Sci USA 100: 5372-7 (2003)). Abnormal protein binding of ATM may cause increased incidence of familial form of breast neoplasms (Cancer Res 63: 3325-33 (2003)). Splice site mutation in the ATM gene causes increased incidence of familial form of ovarian neoplasms (Cancer Res 63: 3325-33 (2003)). Splice site mutation in the ATM gene may correlate with genomic instability associated with colonic neoplasms (Int J Cancer 86: 262-8 (2000)). Deletion mutation in the ATM gene may correlate with genomic instability associated with colonic neoplasms (Int J Cancer 86: 262-8 (2000)). Mutation in the ATM gene causes increased incidence of familial form of ovarian neoplasms (Cancer Res 63: 3325-33 (2003)). Mutation in the ATM gene correlates with decreased response to ionizing radiation associated with chronic B-cell leukemia (Blood 98: 814-22 (2001)). Mutation in the ATM gene causes increased incidence of familial form of breast neoplasms (Cancer Res 63: 3325-33 (2003)). Missense mutation in the ATM gene may correlate with breast neoplasms (PNAS 99: 925-30 (2002)). Induced stimulation of the protein kinase activity of ATM may correlate with increased response to drug associated with myeloid leukemia (Blood 101: 4589-97 (2003)). Nonsense mutation in the ATM gene causes diffuse large-cell lymphoma (Blood 100: 1430-7 (2002)). Decreased protein kinase activity of ATM may cause increased incidence of familial form of breast neoplasms (Proc Natl Acad Sci USA 99: 925-30 (2002)). Missense mutation in the ATM gene may cause increased incidence of non-familial form of breast neoplasms (Proc Natl Acad Sci USA 99: 925-30 (2002)). Missense mutation in the ATM gene causes increased incidence of familial form of breast neoplasms (Cancer Res 63: 3325-33 (2003)). (PhosphoSite®, Cell Signaling Technology (Danvers, Mass.), Human PSD™, Biobase Corporation, (Beverly, Mass.)).

Bcr (P11274), phosphorylated at Y513, is among the proteins listed in this patent. Bcr, Breakpoint cluster region, GTPase-activating protein for p21rac with serine-threonine kinase activity; BCR-ABL gene fusion is associated with several types of leukemia and multiple myeloma, variants may be associated with bipolar disorder. This protein has potential diagnostic and/or therapeutic implications based on the following findings. Translocation of the BCR gene correlates with acute B-cell leukemia (Leukemia 15: 1834-40 (2001)). Amplification of the BCR gene correlates with mycosis fungoides associated with skin neoplasms (Blood 101: 1513-9 (2003)). Amplification of the BCR gene correlates with drug-resistant form of leukemia (Cancer 100: 1459-71 (2004)). Translocation of the BCR gene correlates with early onset form of acute L2 lymphocytic leukemia (Cancer 73: 1526-32 (1994)). Decreased expression of BCR mutant protein may prevent chronic-phase myeloid leukemia (Blood 87: 4770-9 (1996)). Increased expression of BCR mutant protein correlates with early onset form of acute T-cell leukemia (Leukemia 8: 1124-30 (1994)). Translocation of the BCR gene correlates with decreased cell differentiation associated with chronic myeloid leukemia (Leukemia 13: 2007-11 (1999)). Translocation of the BCR gene causes acute lymphocytic leukemia (Leukemia 4: 397-403 (1990)). Translocation of the BCR gene correlates with chronic myeloid leukemia associated with Philadelphia-positive myeloid leukemia (Leukemia 13: 2007-11 (1999)). Decreased expression of BCR mutant protein prevents increased occurrence of recurrence associated with acute L2 lymphocytic leukemia (Blood 100: 2357-66 (2002)). Translocation of the BCR gene correlates with advanced stage or high grade form of acute lymphocytic leukemia (L1) (Leukemia 9: 1689-93 (1995)). Deletion mutation in the BCR gene correlates with chronic myeloid leukemia (Blood 97: 3581-8 (2001)). Translocation of the BCR gene correlates with acute form of leukemia (Leukemia 9: 1483-6 (1995)). Amplification of the BCR gene may correlate with drug-resistant form of leukemia (Blood 95: 1758-66 (2000)). Translocation of the BCR gene correlates with recurrence associated with acute T-cell leukemia (Leukemia 8: 889-94 (1994)). Translocation of the BCR gene correlates with chronic form of Philadelphia-negative myeloid leukemia (Blood 88: 2236-40 (1996)). Translocation of the BCR gene correlates with acute myelocytic leukemia associated with acute L2 lymphocytic leukemia (Cancer 73: 1526-32 (1994)). Decreased expression of BCR mutant protein may cause increased apoptosis associated with chronic myeloid leukemia (Oncogene 21: 5716-24 (2002)). Translocation of the BCR gene correlates with chronic-phase myeloid leukemia (Blood 98: 3778-83 (2001)). Translocation of the BCR gene correlates with Philadelphia chromosome associated with chronic myeloid leukemia (Leukemia 13: 2007-11 (1999)). Translocation of the BCR gene correlates with increased response to drug associated with acute promyelocytic leukemia (Oncogene 22: 6900-8 (2003)). Amplification of the BCR gene correlates with Sezary syndrome associated with skin neoplasms (Blood 101: 1513-9 (2003)). Induced inhibition of BCR mutant protein may prevent decreased apoptosis associated with chronic myeloid leukemia (Blood 91: 641-8 (1998)). Translocation of the BCR gene correlates with pre-B-cell leukemia associated with chronic myeloid leukemia (Leukemia 13: 2007-11 (1999)). Induced inhibition of BCR mutant protein may prevent increased cell proliferation associated with chronic myeloid leukemia (Blood 91: 3414-22 (1998)). Induced inhibition of BCR mutant protein may cause increased apoptosis associated with chronic myeloid leukemia (Leukemia 15: 1537-43 (2001)). Induced inhibition of BCR mutant protein may prevent decreased cell cycle arrest associated with chronic myeloid leukemia (Blood 91: 641-8 (1998)). Decreased expression of BCR mutant protein may cause increased apoptosis associated with acute L2 lymphocytic leukemia (Blood 104: 356-63 (2004)). Increased expression of BCR mutant protein correlates with increased incidence of disease progression associated with chronic myeloid leukemia (Blood 86: 2371-8 (1995)). Mutation in the BCR gene correlates with chronic form of Philadelphia-negative myeloid leukemia (Cancer 75: 464-70 (1995)). Translocation of the BCR gene correlates with decreased incidence of death associated with Philadelphia-positive myeloid leukemia (Leukemia 4: 448-9 (1990)). Methylation of the BCR gene correlates with Philadelphia-negative myeloid leukemia (Leukemia 6: 35-41 (1992)). Translocation of the BCR gene correlates with increased incidence of death associated with acute B-cell leukemia (Blood 102: 2014-20 (2003)). Deletion mutation in the BCR gene correlates with acute L2 lymphocytic leukemia (Blood 97: 3581-8 (2001)). Translocation of the BCR gene correlates with chronic myeloid leukemia (Hum Mol Genet 11: 1391-7 (2002)). Decreased expression of BCR mutant protein may prevent increased incidence of recurrence associated with chronic-phase myeloid leukemia (Blood 93: 284-92 (1999)). Translocation of the BCR gene may cause multiple myeloma (Nucleic Acids Res 28: 4865-72 (2000)). Translocation of the BCR gene correlates with pre-B-cell leukemia (Leukemia 13: 2007-11 (1999)). Translocation of the BCR gene correlates with pre-B-cell leukemia (Leukemia 15: 1834-40 (2001)). Decreased expression of BCR mutant protein may prevent recurrence associated with Philadelphia-negative myeloid leukemia (Leukemia 13: 999-1008 (1999)). Translocation of the BCR gene may correlate with Philadelphia chromosome associated with acute T-cell leukemia (Leukemia 8: 889-94 (1994)). Decreased expression of BCR mutant protein may prevent increased incidence of recurrence associated with chronic-phase myeloid leukemia (Blood 87: 2588-93 (1996)). Translocation of the BCR gene correlates with chronic form of Philadelphia-negative myeloid leukemia (Leukemia 6: 385-92 (1992)). Translocation of the BCR gene correlates with Philadelphia chromosome associated with pre-B-cell leukemia (Leukemia 13: 2007-11 (1999)). Alternative form of BCR mutant protein correlates with increased response to drug associated with Philadelphia-positive myeloid leukemia (Leukemia 6: 948-51 (1992)). Translocation of the BCR gene correlates with late onset form of acute B-cell leukemia (Leukemia 7: 10547 (1993)). Translocation of the BCR gene correlates with chronic myeloid leukemia associated with pre-B-cell leukemia (Leukemia 13: 2007-11 (1999)). Alternative form of BCR mutant protein correlates with decreased isotype switching associated with chronic myeloid leukemia (Leukemia 13: 2007-11 (1999)). Translocation of the BCR gene correlates with decreased isotype switching associated with chronic myeloid leukemia (Leukemia 13: 2007-11 (1999)). (PhosphoSite®, Cell Signaling Technology (Danvers, Mass.), Human PSD™, Biobase Corporation, (Beverly, Mass.)).

Btk (Q06187), phosphorylated at Y39, Y333, is among the proteins listed in this patent. Btk, Bruton agammaglobulinemia tyrosine kinase, functions in pre-B cell receptor signaling and B cell development; gene mutation is associated with X-linked agammaglobulinemia (XLA), mouse Btk gene mutation is associated with X-linked immunodeficiency (Xid). This protein has potential diagnostic and/or therapeutic implications based on the following findings. Mutation in the BTK gene causes immunologic deficiency syndromes associated with agammaglobulinemia (Proc Natl Acad Sci USA 91: 9062-6 (1994)). Mutation in the BTK gene causes abnormal B cell differentiation associated with agammaglobulinemia (Nature 361: 226-33 (1993)). Mutation in the BTK gene causes agammaglobulinemia (Nucleic Acids Res 24: 160-5 (1996)). Splice site mutation in the BTK gene causes agammaglobulinemia (FEBS Lett 346: 165-70 (1994)). Splice site mutation in the BTK gene causes agammaglobulinemia (Hum Mol Genet 4: 693-700 (1995)). Mutation in the BTK gene causes immunologic deficiency syndromes associated with agammaglobulinemia (Proc Natl Acad Sci USA 91: 9062-6 (1994)). Insertion mutation in the BTK gene causes agammaglobulinemia (Hum Mol Genet 3: 161-6 (1994)). Missense mutation in the protein kinase domain of BTK causes agammaglobulinemia (Hum Mol Genet 4: 693-700 (1995)). Missense mutation in the BTK gene causes agammaglobulinemia (PNAS 91: 9062-6 (1994)). Splice site mutation in the BTK gene causes agammaglobulinemia (Am J Hum Genet 60: 798-807 (1997)). Missense mutation in the BTK gene causes agammaglobulinemia (Proc Natl Acad Sci USA 91: 9062-6 (1994)). Nonsense mutation in the BTK gene causes immunologic deficiency syndromes associated with agammaglobulinemia (Mol Med 2: 619-23 (1996)). Nonsense mutation in the BTK gene causes agammaglobulinemia (Hum Mol Genet 3: 1751-6 (1994)). Deletion mutation in the BTK gene causes less severe form of agammaglobulinemia (Clin Exp Immunol 107: 235-40 (1997)). Mutation in the BTK gene causes agammaglobulinemia (Am J Hum Genet 62: 1034-43 (1998)). Mutation in the BTK gene causes immunologic deficiency syndromes associated with agammaglobulinemia (PNAS 91: 9062-6 (1994)). Nonsense mutation in the BTK gene causes agammaglobulinemia (Mol Med 2: 619-23 (1996)). Decreased phosphatidylinositol-3,4,5-triphosphate binding of BTK may cause agammaglobulinemia (EMBO J. 16: 3396-404 (1997)). Decreased phosphatidylinositol-3,4,5-tri phosphate binding of BTK may cause agammaglobulinemia (EMBO J 16: 3396-404 (1997)). Decreased expression of BTK in monocytes correlates with abnormal B-lymphocytes function associated with agammaglobulinemia (Blood 91: 595-602 (1998)). Missense mutation in the protein kinase domain of BTK causes agammaglobulinemia (Proc Natl Acad Sci USA 91: 12803-7 (1994)). Nonsense mutation in the BTK gene causes agammaglobulinemia (PNAS 91: 9062-6 (1994)). Absence of the protein binding of BTK may cause abnormal signal transduction associated with agammaglobulinemia (J Exp Med 180: 461-70 (1994)). Decreased expression of BTK in B-lymphocytes correlates with agammaglobulinemia (Cell 72: 279-90 (1993)). Insertion mutation in the BTK gene causes agammaglobulinemia (Blood 96: 610-7 (2000)). Deletion mutation in the BTK gene causes agammaglobulinemia (Blood 96: 610-7 (2000)). Absence of the protein kinase activity of BTK causes agammaglobulinemia (Blood 88: 561-73 (1996)). Decreased phosphatidylinositol-3,4,5-triphosphate binding of BTK may cause agammaglobulinemia (EMBO 16: 3396-404 (1997)). Missense mutation in the protein kinase domain of BTK causes agammaglobulinemia (PNAS 91: 12803-7 (1994)). Missense mutation in the PH domain of BTK causes agammaglobulinemia (EMBO J. 16: 3396-404 (1997)). Mutation in the BTK gene causes abnormal B cell differentiation associated with agammaglobulinemia (Hum Mol Genet 3: 161-6 (1994)). Frameshift mutation in the BTK gene causes agammaglobulinemia (FEBS Lett 346: 165-70 (1994)). Mutation in the BTK gene causes agammaglobulinemia (Nucleic Acids Res 26: 242-7 (1998)). Missense mutation in the PH domain of BTK causes agammaglobulinemia (EMBO 16: 3396-404 (1997)). Missense mutation in the BTK gene causes agammaglobulinemia (Hum Mol Genet 3: 1751-6 (1994)). Deletion mutation in the BTK gene causes agammaglobulinemia (Proc Natl Acad Sci USA 91: 9062-6 (1994)). Deletion mutation in the BTK gene causes agammaglobulinemia (Proc Natl Acad Sci USA 91: 9062-6 (1994)). Insertion mutation in the BTK gene causes agammaglobulinemia (Hum Mol Genet 3: 79-83 (1994)). Decreased expression of BTK mRNA correlates with agammaglobulinemia (Blood 88: 561-73 (1996)). Nonsense mutation in the BTK gene causes agammaglobulinemia (Proc Natl Acad Sci USA 91: 9062-6 (1994)). Point mutation in the BTK gene causes agammaglobulinemia (Hum Mol Genet 4: 693-700 (1995)). Deletion mutation in the SH3 domain of BTK causes abnormal B cell differentiation associated with agammaglobulinemia (J Exp Med 180: 461-70 (1994)). Missense mutation in the BTK gene causes agammaglobulinemia (FEBS Lett 413: 205-10 (1997)). Frameshift mutation in the BTK gene causes agammaglobulinemia (PNAS 91: 9062-6 (1994)). Nonsense mutation in the BTK gene causes agammaglobulinemia (Proc Natl Acad Sci USA 91: 9062-6 (1994)). Splice site mutation in the BTK gene causes agammaglobulinemia (Hum Mol Genet 4: 51-8 (1995)). Nonsense mutation in the BTK gene causes agammaglobulinemia (Hum Mol Genet 3: 161-6 (1994)). Mutation in the BTK gene causes immunologic deficiency syndromes associated with agammaglobulinemia (Clin Exp Immunol 120: 512-7 (2000)). Missense mutation in the BTK gene causes agammaglobulinemia (Proc Natl Acad Sci USA 91: 9062-6 (1994)). Missense mutation in the SH2 domain of BTK causes agammaglobulinemia (J Immunol 164: 4170-7 (2000)). Missense mutation in the protein kinase domain of BTK causes agammaglobulinemia (Clin Exp Immunol 120: 346-50 (2000)). Missense mutation in the PH domain of BTK causes agammaglobulinemia (Blood 88: 561-73 (1996)). Missense mutation in the BTK gene causes agammaglobulinemia (J Immunol 161: 3925-9 (1998)). Mutation in the BTK gene causes agammaglobulinemia (J Immunol 167: 4038-45 (2001)). Decreased protein kinase activity of BTK causes agammaglobulinemia (Clin Exp Immunol 120: 346-50 (2000)). Missense mutation in the BTK gene causes agammaglobulinemia (Hum Mol Genet 4: 51-8 (1995)). Mutation in the BTK gene causes agammaglobulinemia (Nucleic Acids Res 25: 166-71 (1997)). Deletion mutation in the BTK gene causes late onset form of agammaglobulinemia (Clin Exp Immunol 107: 235-40 (1997)). Frameshift mutation in the BTK gene causes agammaglobulinemia (Hum Mol Genet 4: 51-8 (1995)). Frameshift mutation in the BTK gene causes agammaglobulinemia (Proc Natl Acad Sci USA 91: 9062-6 (1994)). Missense mutation in the PH domain of BTK causes agammaglobulinemia (EMBO J 16: 3396-404 (1997)). Mutation in the BTK gene causes agammaglobulinemia (Hum Mol Genet 3: 79-83 (1994)). Missense mutation in the BTK gene causes agammaglobulinemia (Blood 96: 610-7 (2000)). Deletion mutation in the BTK gene causes agammaglobulinemia (PNAS 91: 9062-6 (1994)). Frameshift mutation in the BTK gene causes agammaglobulinemia (Proc Natl Acad Sci USA 91: 9062-6 (1994)). Missense mutation in the protein kinase domain of BTK causes agammaglobulinemia (Proc Natl Acad Sci USA 91: 12803-7 (1994)). Deletion mutation in the BTK gene causes agammaglobulinemia (Hum Mol Genet 4: 693-700 (1995)). Splice site mutation in the BTK gene causes agammaglobulinemia (Hum Mol Genet 3: 1751-6 (1994)). Nonsense mutation in the BTK gene causes agammaglobulinemia (Hum Mol Genet 4: 51-8 (1995)). Decreased protein-tyrosine kinase activity of BTK causes agammaglobulinemia (Clin Exp Immunol 107: 235-40 (1997)). Missense mutation in the protein kinase domain of BTK causes agammaglobulinemia (Mol Med 6: 104-13 (2000)). Frameshift mutation in the BTK gene causes agammaglobulinemia (Hum Mol Genet 3: 1751-6 (1994)). Missense mutation in the SH2 domain of BTK causes agammaglobulinemia (Hum Mol Genet 3: 161-6 (1994)). (PhosphoSite®, Cell Signaling Technology (Danvers, Mass.), Human PSD™, Biobase Corporation, (Beverly, Mass.)).

Bub1 (O43683), phosphorylated at Y219, is among the proteins listed in this patent. Bub1, Budding uninhibited by benzimidazoles 1 homolog, acts in spindle assembly checkpoint and chromosome congression, may regulate vesicular traffic; mutations are associated with lung cancer, T cell leukemia and colorectal cancer cell chromosomal instability. This protein has potential diagnostic and/or therapeutic implications based on the following findings. Missense mutation in the BUB1 gene may cause abnormal mitotic sister chromatid segregation associated with colorectal neoplasms (Nature 392: 300-303 (1998)). Decreased expression of BUB1 mRNA may cause increased occurrence of neoplasm metastasis associated with colorectal neoplasms (Cancer Res 62: 13-7 (2002)). Missense mutation in the BUB1 gene may cause increased occurrence of malignant form of colorectal neoplasms (Cancer Res 62: 13-7 (2002)). Locus instability of BUB1 may cause colorectal neoplasms (Cancer Res 60: 4349-52 (2000)). Deletion mutation in the BUB1 gene may cause chromosome aberrations associated with acute HTLV-1-associated leukemia (Cancer Lett 158: 141-50 (2000)). Splice site mutation in the BUB1 gene may cause abnormal mitotic sister chromatid segregation associated with colorectal neoplasms (Nature 392: 300-303 (1998)). Missense mutation in the BUB1 gene may cause increased occurrence of neoplasm metastasis associated with colorectal neoplasms (Cancer Res 62: 13-7 (2002)). Point mutation in the BUB1 gene may cause chromosome aberrations associated with acute HTLV-1-associated leukemia (Cancer Lett 158: 141-50 (2000)). Missense mutation in the BUB1 gene may cause recurrence associated with colorectal neoplasms (Cancer Res 62: 13-7 (2002)). Decreased expression of BUB1 mRNA may cause increased occurrence of malignant form of colorectal neoplasms (Cancer Res 62: 13-7 (2002)). Splice site mutation in the BUB1 gene may cause abnormal mitotic checkpoint associated with colorectal neoplasms (Nature 392: 300-303 (1998)). Missense mutation in the BUB1 gene may cause abnormal mitotic checkpoint associated with colorectal neoplasms (Nature 392: 300-303 (1998)). Decreased expression of BUB1 mRNA may cause recurrence associated with colorectal neoplasms (Cancer Res 62: 13-7 (2002)). (PhosphoSite®, Cell Signaling Technology (Danvers, Mass.), Human PSD™, Biobase Corporation, (Beverly, Mass.)).

CD34 (P28906), phosphorylated at Y330, is among the proteins listed in this patent. CD34, CD34 antigen, a transmembrane sialomucin associated with hematopoietic stem cells and an L-selectin ligand on high endothelial venules, transduces signals that regulate cytoadhesion of hematopoietic cells, may play a role in early stages of hematopoiesis. This protein has potential diagnostic and/or therapeutic implications based on the following findings. Abnormal expression of CD34 protein may correlate with acute myelocytic leukemia (Blood 86: 60-5 (1995)). Increased expression of CD34 in hematopoietic stem cells may correlate with less severe form of HIV infections (Blood 86: 1749-56 (1995)). Abnormal expression of CD34 protein may correlate with chronic myeloid leukemia (Blood 86: 60-5 (1995)). (PhosphoSite®, Cell Signaling Technology (Danvers, Mass.), Human PSD™, Biobase Corporation, (Beverly, Mass.)).

CLH-17 (Q00610), phosphorylated at Y1095, Y1205, Y1210, is among the proteins listed in this patent. CLH-17, Clathrin heavy polypeptide Hc, binds huntingtin interacting protein 1 (HIP1), involved in endocytosis, may bind to endocytic proteins; gene fusion to ALK is associated with inflammatory myofibroblastic tumor and large B-cell lymphoma. This protein has potential diagnostic and/or therapeutic implications based on the following findings. Translocation of the CLTC gene correlates with B-cell lymphoma associated with diffuse large-cell lymphoma (Blood 102: 2568-73 (2003)). Translocation of the CLTC gene correlates with B-cell lymphoma (Blood 102: 2638-41 (2003)). Translocation of the CLTC gene correlates with diffuse large-cell lymphoma associated with B-cell lymphoma (Blood 102: 2568-73 (2003)). (PhosphoSite®, Cell Signaling Technology (Danvers, Mass.), Human PSD™, Biobase Corporation, (Beverly, Mass.)).

CR2 (P20023), phosphorylated at Y1029, is among the proteins listed in this patent. CR2, Complement receptor 2, binds to the breakdown products of complement C3 and interacts with CD23 (FCER2); altered expression is associated with various diseases; inhibition by blocking antibody may be therapeutic for HIV infection. This protein has potential diagnostic and/or therapeutic implications based on the following findings. Decreased expression of CR2 protein correlates with chronic B-cell leukemia (Clin Exp Immunol 83: 423-9 (1991)). Increased expression of CR2 in T-lymphocytes correlates with glomerulonephritis associated with systemic lupus erythematosus (Clin Exp Immunol 90: 235-44 (1992)). Increased expression of CR2 protein may cause increased cell-cell adhesion associated with multiple myeloma (Blood 85: 3704-12 (1995)). Increased presence of CR2 antibody may prevent increased entry of virus into host cell associated with HIV infections (Eur J Immunol 33: 2098-107 (2003)). Increased expression of CR2 in B-lymphocytes correlates with asthma (Clin Exp Immunol 94: 337-40 (1993)). Decreased expression of CR2 in B-lymphocytes correlates with systemic lupus erythematosus (Clin Exp Immunol 101: 60-5 (1995)). Increased expression of CR2 protein correlates with interstitial nephritis associated with Epstein-Barr virus infections (J Clin Invest 104: 1673-81 (1999)). Increased expression of CR2 in B-lymphocytes may correlate with abnormal immune response associated with asthma (Eur J Immunol 24: 1109-14 (1994)). Decreased expression of CR2 protein correlates with chronic lymphocytic leukemia (Clin Exp Immunol 102: 575-81 (1995)). Decreased expression of CR2 in T-lymphocytes correlates with HIV infections (Immunology 75: 59-65 (1992)). (PhosphoSite®, Cell Signaling Technology (Danvers, Mass.), Human PSD™, Biobase Corporation, (Beverly, Mass.)).

CrkL (P46109), phosphorylated at Y48, Y92, Y198, is among the proteins listed in this patent. CrkL, V-crk sarcoma virus CT10 oncogene homolog (avian)-like, SH2-SH3 adaptor protein and transcription cofactor, activates RAS and JUN kinase pathways, associated with chronic myelogenous leukemia; mutations in mouse Crkl mimic DiGeorge syndrome. This protein has potential diagnostic and/or therapeutic implications based on the following findings. Decreased expression of CRKL protein may prevent increased cell proliferation associated with Philadelphia-positive myeloid leukemia (Biochem Biophys Res Commun 235: 383-8 (1997)). Increased phosphorylation of CRKL correlates with Philadelphia-positive myeloid leukemia (Blood 84: 2912-8 (1994)). Increased phosphorylation of CRKL may correlate with decreased response to drug associated with chronic myeloid leukemia (Leukemia 18: 401-8 (2004)). Abnormal SH3/SH2 adaptor activity of CRKL may cause abnormal cell adhesion associated with Philadelphia-positive myeloid leukemia (JBC 270: 29145-50 (1995)). Decreased SH3/SH2 adaptor activity of CRKL may prevent increased cell proliferation associated with chronic myeloid leukemia (FASEB 14: 1529-38 (2000)). CRKL map position correlates with DiGeorge syndrome (Nat Genet. 27: 293-8 (2001)). Decreased phosphorylation of CRKL may correlate with increased response to drug associated with chronic myeloid leukemia (Blood 104: 509-18 (2004)). Abnormal SH3/SH2 adaptor activity of CRKL may cause abnormal cell adhesion associated with Philadelphia-positive myeloid leukemia (J Biol Chem 270: 29145-50 (1995)). Abnormal SH3/SH2 adaptor activity of CRKL may cause abnormal intracellular signaling cascade associated with Philadelphia-positive myeloid leukemia (J Biol Chem 270: 21468-71 (1995)). Increased phosphorylation of CRKL correlates with Philadelphia-positive myeloid leukemia (Blood 84: 1731-6 (1994)). Increased phosphorylation of CRKL correlates with Philadelphia-positive myeloid leukemia (Blood 88: 4304-13 (1996)). Increased phosphorylation of CRKL correlates with Philadelphia-positive myeloid leukemia (JBC 269: 22925-8 (1994)). Increased phosphorylation of CRKL correlates with Philadelphia-positive myeloid leukemia (J Biol Chem 269: 22925-8 (1994)). Abnormal SH3/SH2 adaptor activity of CRKL may cause abnormal intracellular signaling cascade associated with Philadelphia-positive myeloid leukemia (JBC 270: 21468-71 (1995)). Decreased SH3/SH2 adaptor activity of CRKL may prevent increased cell proliferation associated with chronic myeloid leukemia (FASEB J 14: 1529-38 (2000)). (PhosphoSite®, Cell Signaling Technology (Danvers, Mass.), Human PSD™, Biobase Corporation, (Beverly, Mass.)).

CRMP-2 (Q16555), phosphorylated at Y499, is among the proteins listed in this patent. CRMP-2, Dihydropyrimidinase-like 2, binds tubulin and axon growth cone proteins, regulates microtubule formation, acts in neuronal growth cone collapse and axonal growth, abnormal expression or modification is linked to neuroinflammatory and Alzheimer diseases. This protein has potential diagnostic and/or therapeutic implications based on the following findings. Increased phosphorylation of DPYSL2 correlates with Alzheimer disease (Biochemistry 39: 4267-75 (2000)). Increased oxidation of DPYSL2 correlates with Alzheimer disease (J Neurochem 82: 1524-32 (2002)). Increased phosphorylation of DPYSL2 correlates with Alzheimer disease (Biochemistry Usa 39: 4267-75 (2000)). (PhosphoSite®, Cell Signaling Technology (Danvers, Mass.), Human PSD™, Biobase Corporation, (Beverly, Mass.)).

CSK (P41240), phosphorylated at Y416, is among the proteins listed in this patent. CSK, C-src tyrosine kinase, a protein tyrosine kinase with SH2 and SH3 domains, inactivates the c-src (SRC) oncoprotein, regulates receptor signaling pathways and possibly T-cell activation, and acts as a tumor antigen in carcinomas. This protein has potential diagnostic and/or therapeutic implications based on the following findings. Decreased expression of CSK protein correlates with carcinoma tumors associated with colorectal neoplasms (Cancer 92: 61-70 (2001)). Increased presence of CSK autoimmune antibody correlates with adenocarcinoma (Cancer Res 61: 1415-20 (2001)). Abnormal expression of CSK protein may cause abnormal cell-cell adhesion associated with colonic neoplasms (Oncogene 23: 289-97 (2004)). Abnormal expression of CSK protein may cause abnormal integrin-mediated signaling pathway associated with colonic neoplasms (Oncogene 23: 289-97 (2004)). Increased expression of CSK protein correlates with adenocarcinoma (Cancer Res 61: 1415-20 (2001)). Increased protein binding of CSK correlates with increased intracellular signaling cascade associated with prostatic neoplasms (Int J Cancer 68: 164-71 (1996)). Increased protein binding of CSK correlates with malignant form of prostatic neoplasms (Int J Cancer 68: 164-71 (1996)). Abnormal expression of CSK protein may cause abnormal cell migration associated with colonic neoplasms (Oncogene 23: 289-97 (2004)). Increased protein binding of CSK correlates with increased carcinoma associated with prostatic neoplasms (Int J Cancer 68: 164-71 (1996)). Increased presence of CSK autoimmune antibody correlates with carcinoma associated with neoplasms (Cancer Res 61: 1415-20 (2001)). (PhosphoSite®, Cell Signaling Technology (Danvers, Mass.), Human PSD™, Biobase Corporation, (Beverly, Mass.)).

DNA-PK (P78527), phosphorylated at Y779, Y883, Y2936, is among the proteins listed in this patent. DNA-PK, DNA-dependent protein kinase catalytic subunit, a DNA-binding protein kinase involved in DNA double-strand break repair, V(D)J recombination, and transcriptional regulation, phosphorylates and activates AKT; mouse Prkdc deficiency is associated with SCID. This protein has potential diagnostic and/or therapeutic implications based on the following findings. Decreased expression of PRKDC mRNA may prevent decreased response to radiation associated with prostatic neoplasms (Cancer Res 63: 1550-4 (2003)). Mutation in the PRKDC gene correlates with colorectal neoplasms (Cancer Res 62: 1284-8 (2002)). Decreased expression of PRKDC protein may correlate with increased response to radiation associated with lung neoplasms (Eur J Cancer 35: 111-6 (1999)). Decreased expression of PRKDC mRNA may prevent decreased response to radiation associated with non-small-cell lung carcinoma (Cancer Res 62: 6621-4 (2002)). Increased cleavage of PRKDC may prevent multiple myeloma (Blood 101: 1530-4 (2003)). Abnormal expression of PRKDC mRNA may correlate with chronic lymphocytic leukemia (Anticancer Res 22: 1787-93 (2002)). Single nucleotide polymorphism in the PRKDC gene correlates with increased occurrence of disease susceptibility associated with breast neoplasms (Cancer Res 63: 2440-6 (2003)). Decreased proteolysis of PRKDC may correlate with drug-resistant form of Burkitt Lymphoma (Int J Cancer 77: 755-62 (1998)). MRNA instability of PRKDC may cause decreased double-strand break repair associated with glioma (Oncogene 18: 1361-8 (1999)). Gene instability of PRKDC may correlate with colorectal neoplasms (Hum Mol Genet 10: 513-8 (2001)). (PhosphoSite®, Cell Signaling Technology (Danvers, Mass.), Human PSD™, Biobase Corporation, (Beverly, Mass.)).

eIF2B (P20042), phosphorylated at Y298, is among the proteins listed in this patent. eIF2B, Eukaryotic translation initiation factor 2 subunit 2 beta 38 kDa, beta subunit of eIF2, which is a translation initiation factor involved in the initiation of protein synthesis; mutations are linked to leukoencephalopathy with vanishing white matter. This protein has potential diagnostic and/or therapeutic implications based on the following findings (PhosphoSite®, Cell Signaling Technology (Danvers, Mass.), Human PSD™, Biobase Corporation, (Beverly, Mass.)).

ENO2 (P09104), phosphorylated at Y24, is among the proteins listed in this patent. ENO2, Neuron-specific enolase (gamma enolase), catalyzes conversion of 2-phospho-D-glycerate to phosphoenolpyruvate in glycolysis, may be involved in neuronal differentiation; altered expression is seen in breast cancer, lung cancer, and multiple sclerosis. This protein has potential diagnostic and/or therapeutic implications based on the following findings. Abnormal expression of ENO2 in serum correlates with decreased response to drug associated with small cell carcinoma (Cancer 82: 1049-55 (1998)). Increased expression of ENO2 protein correlates with leiomyosarcoma associated with ovarian neoplasms (Anticancer Res 23: 3433-6 (2003)). Increased expression of ENO2 protein may correlate with carcinoma tumors associated with pancreatic neoplasms (Cancer 70: 1514-9 (1992)). Increased expression of ENO2 in serum correlates with small cell carcinoma (Anticancer Res 11: 2107-10 (1991)). Increased expression of ENO2 in serum correlates with disease progression associated with small cell carcinoma (Cancer 72: 418-25 (1993)). Increased expression of ENO2 protein correlates with carcinoma tumors associated with breast neoplasms (Br J Cancer 82: 20-7 (2000)). Increased expression of ENO2 in serum correlates with small cell carcinoma (Eur J Cancer: 198-202 (1993)). Increased expression of ENO2 protein may correlate with melanoma (Eur J Cancer 31: 1898-902 (1995)). Decreased expression of ENO2 protein correlates with decreased occurrence of death associated with non-small-cell lung carcinoma (Anticancer Res 22: 1083-9 (2002)). Increased expression of ENO2 in serum correlates with adenocarcinoma tumors associated with lung neoplasms (Eur J Cancer: 198-202 (1993)). Increased expression of ENO2 protein correlates with non-small-cell lung carcinoma (Anticancer Res 23: 885-93 (2003)). Increased expression of ENO2 in serum correlates with increased occurrence of death associated with small cell carcinoma (Br J Cancer 67: 760-6 (1993)). Increased expression of ENO2 in serum correlates with neoplasm metastasis associated with non-small-cell lung carcinoma (Br J Cancer 84: 903-9 (2001)). (PhosphoSite®, Cell Signaling Technology (Danvers, Mass.), Human PSD™, Biobase Corporation, (Beverly, Mass.)).

ERK2 (P28482), phosphorylated at Y35, is among the proteins listed in this patent. ERK2, Mitogen-activated protein kinase 1, a serine-threonine kinase effector of the RAS-MAP kinase pathway, translocates to the nucleus to mediate transcription when activated, involved in the regulation of cell growth, differentiation, migration and apoptosis. This protein has potential diagnostic and/or therapeutic implications based on the following findings. Decreased nucleus localization of MAPK1 may correlate with increased transforming growth factor beta receptor signaling pathway associated with pancreatic neoplasms (Oncogene 19: 4531-41 (2000)). Decreased expression of MAPK1 in hippocampus correlates with depression (J Neurochem 77: 916-28 (2001)). Insertion mutation in the MAPK1 gene correlates with hepatitis B associated with hepatocellular carcinoma (Oncogene 22: 3911-6 (2003)). Induced stimulation of the protein kinase activity of MAPK1 may cause increased apoptosis associated with lung neoplasms (Oncogene 22: 5427-35 (2003)). Increased nucleus localization of MAPK1 may cause decreased induction of apoptosis in response to chemical stimulus associated with leukemia (J Biol Chem 279: 32813-23 (2004)). Induced stimulation of the MAP kinase 1 activity of MAPK1 may cause increased actin filament organization associated with ovarian neoplasms (J Biol Chem 280: 11961-72 (2005)). Increased phosphorylation of MAPK1 may cause increased signal transduction associated with ovarian neoplasms (J Biol Chem 280: 11961-72 (2005)). Increased phosphorylation of MAPK1 may cause increased cell death associated with breast neoplasms (FEBS Lett 458: 137-40 (1999)). Increased MAP kinase activity of MAPK1 correlates with malignant form of non-small-cell lung carcinoma (Br J Cancer 90: 1047-52 (2004)). Increased cytosol localization of MAPK1 may cause decreased induction of apoptosis associated with leukemia (JBC 279: 32813-23 (2004)). Decreased phosphorylation of MAPK1 may cause increased apoptosis associated with colonic neoplasms (Br J Cancer 82: 905-12 (2000)). Induced inhibition of the MAP kinase 1 activity of MAPK1 may prevent drug-resistant form of multiple myeloma (Blood 101: 703-5 (2003)). Increased MAP kinase activity of MAPK1 correlates with advanced stage or high grade form of non-small-cell lung carcinoma (Br J Cancer 90: 1047-52 (2004)). Induced stimulation of the MAP kinase 1 activity of MAPK1 may cause increased signal transduction associated with ovarian neoplasms (J Biol Chem 280: 11961-72 (2005)). Induced inhibition of the MAP kinase 1 activity of MAPK1 may correlate with increased transforming growth factor beta receptor signaling pathway associated with pancreatic neoplasms (Oncogene 19: 4531-41 (2000)). Increased cytosol localization of MAPK1 may cause decreased induction of apoptosis associated with leukemia (J Biol Chem 279: 32813-23 (2004)). Increased nucleus localization of MAPK1 may cause decreased induction of apoptosis in response to chemical stimulus associated with leukemia (JBC 279: 32813-23 (2004)). Increased phosphorylation of MAPK1 may cause increased signal transduction associated with ovarian neoplasms (JBC 280: 11961-72 (2005)). Increased expression of MAPK1 protein correlates with breast neoplasms (Anticancer Res 19: 731-40 (1999)). Insertion mutation in the MAPK1 gene correlates with hepatitis B associated with liver neoplasms (Oncogene 22: 3911-6 (2003)). Decreased expression of MAPK1 in frontal cortex correlates with depression (J Neurochem 77: 916-28 (2001)). Abnormal expression of MAPK1 mRNA may correlate with acute myelocytic leukemia (Oncogene 23: 9381-91 (2004)). Increased expression of MAPK1 protein may correlate with hepatocellular carcinoma associated with liver neoplasms (Biochem Biophys Res Commun 236: 54-8 (1997)). Increased MAP kinase 1 activity of MAPK1 may correlate with hepatocellular carcinoma associated with liver neoplasms (Biochem Biophys Res Commun 236: 54-8 (1997)). Increased phosphorylation of MAPK1 may cause increased actin filament organization associated with ovarian neoplasms (JBC 280: 11961-72 (2005)). Decreased phosphorylation of MAPK1 may cause decreased cell proliferation associated with colonic neoplasms (Br J Cancer 82: 905-12 (2000)). Decreased phosphorylation of MAPK1 may correlate with drug-resistant form of prostatic neoplasms (Cancer Res 61: 6060-3 (2001)). Decreased protein kinase activity of MAPK1 correlates with carcinoma associated with colorectal neoplasms (Gut 44: 834-8 (1999)). Induced stimulation of the MAP kinase 1 activity of MAPK1 may cause increased signal transduction associated with ovarian neoplasms (JBC 280: 11961-72 (2005)). Increased MAP kinase activity of MAPK1 correlates with increased severity of non-small-cell lung carcinoma associated with lung neoplasms (Br J Cancer 90: 1047-52 (2004)). Increased phosphorylation of MAPK1 may cause increased actin filament organization associated with ovarian neoplasms (J Biol Chem 280: 11961-72 (2005)). Increased tyrosine phosphorylation of MAPK1 may correlate with increased cytokine and chemokine mediated signaling pathway associated with multiple myeloma (Blood 89: 261-71 (1997)). Decreased protein kinase activity of MAPK1 correlates with adenoma associated with colorectal neoplasms (Gut 44: 834-8 (1999)). Induced stimulation of the MAP kinase 1 activity of MAPK1 may cause increased actin filament organization associated with ovarian neoplasms (JBC 280: 11961-72 (2005)). Induced inhibition of the integrin binding of MAPK1 may prevent disease progression associated with colonic neoplasms (Br J Cancer 87: 348-51 (2002)). Increased expression of MAPK1 protein correlates with increased activation of MAPK activity associated with hepatocellular carcinoma (Biochem Biophys Res Commun 236: 54-8 (1997)). Induced inhibition of the MAP kinase 1 activity of MAPK1 may prevent abnormal cell proliferation associated with multiple myeloma (Blood 101: 703-5 (2003)). (PhosphoSite®, Cell Signaling Technology (Danvers, Mass.), Human PSD™, Biobase Corporation, (Beverly, Mass.)).

EWS (Q01844), phosphorylated at Y417, is among the proteins listed in this patent. EWS, Ewing sarcoma breakpoint region 1, a transcriptional regulator that binds RNA and may function in mRNA processing, signal transduction, or brain development, involved in many cancer-related translocation-fusion events with transcription factors. This protein has potential diagnostic and/or therapeutic implications based on the following findings. EWSR1 mutant protein causes increased transcription initiation associated with Ewing's sarcoma (Oncogene 24: 2715-22 (2005)). Abnormal expression of EWSR1 protein may cause increased cell proliferation associated with chondrosarcoma (Cancer Res 63: 449-54 (2003)). EWSR1 mutant protein may cause increased transcription initiation associated with Ewing's sarcoma (Cancer Res 63: 8338-44 (2003)). EWSR1 mutant protein causes abnormal regulation of transcription associated with Ewing's sarcoma (Oncogene 20: 626-33 (2001)). Translocation of the EWSR1 gene correlates with giant cell tumor of bone associated with bone neoplasms (Int J Cancer 87: 328-35 (2000)). Translocation of the EWSR1 gene may cause malignant form of melanoma (Oncogene 10: 1749-56 (1995)). Translocation of the EWSR1 gene correlates with melanoma tumors associated with soft tissue neoplasms (Nat Genet 4: 341-5 (1993)). Translocation of the EWSR1 gene may cause Ewing's sarcoma associated with bone neoplasms (Cancer Res 60: 1536-40 (2000)). EWSR1 mutant protein may cause abnormal regulation of transcription associated with Ewing's sarcoma (Oncogene 22: 1-9 (2003)). EWSR1 mutant protein may cause abnormal regulation of transcription associated with Ewing's sarcoma (Oncogene 21: 8302-9 (2002)). Translocation of the EWSR1 gene correlates with chondrosarcoma (Cancer Res 63: 449-54 (2003)). Translocation of the EWSR1 gene correlates with clear cell sarcoma associated with soft tissue neoplasms (Oncogene 20: 6653-9 (2001)). Decreased expression of EWSR1 protein may prevent increased cell proliferation associated with primitive neuroectodermal tumors (J Clin Invest 99: 239-47 (1997)). Translocation of the EWSR1 gene may cause abnormal transcription, DNA-dependent associated with melanoma (Oncogene 10: 1749-56 (1995)). Translocation of the EWSR1 gene correlates with neuroblastoma (PNAS 93: 1038-43 (1996)). Translocation of the EWSR1 gene correlates with acute form of leukemia (Cancer Res 62: 5408-12 (2002)). Translocation of the EWSR1 gene correlates with small cell carcinoma (Oncogene 19: 3799-804 (2000)). Induced inhibition of EWSR1 protein may cause increased apoptosis associated with clear cell sarcoma (J Biol Chem 274: 34811-8 (1999)). Translocation of the EWSR1 gene correlates with peripheral primitive neuroectodermal tumors (Cancer Res 58: 2469-76 (1998)). Translocation of the EWSR1 gene correlates with neuroblastoma (Proc Natl Acad Sci USA 93: 1038-43 (1996)). EWSR1 mutant protein may cause abnormal regulation of transcription associated with Ewing's sarcoma (Oncogene 20: 3258-65 (2001)). Translocation of the EWSR1 gene causes abnormal regulation of transcription associated with Ewing's sarcoma (Oncogene 20: 626-33 (2001)). Translocation of the EWSR1 gene correlates with Ewing's sarcoma (Cytogenet Cell Genet 82: 278-83 (1998)). Translocation of the EWSR1 gene may correlate with chondrosarcoma (Cancer 83: 1504-21 (1998)). Translocation of the EWSR1 gene may cause increased transcription, DNA-dependent associated with melanoma (Oncogene 12: 159-67 (1996)). Translocation of the EWSR1 gene correlates with clear cell adenocarcinoma associated with soft tissue neoplasms (Cancer Res 64: 3395-405 (2004)). EWSR1 mutant protein may cause abnormal regulation of transcription associated with Ewing's sarcoma (Oncogene 24: 2512-24 (2005)). Translocation of the EWSR1 gene may cause abnormal regulation of transcription associated with Ewing's sarcoma (Oncogene 22: 1-9 (2003)). Translocation of the EWSR1 gene correlates with neuroblastoma tumors associated with nose neoplasms (Proc Natl Acad Sci USA 93: 1038-43 (1996)). Translocation of the EWSR1 gene may cause abnormal regulation of transcription associated with Ewing's sarcoma (Oncogene 20: 3258-65 (2001)). Induced inhibition of EWSR1 protein may cause increased apoptosis associated with clear cell sarcoma (JBC 274: 34811-8 (1999)). Translocation of the EWSR1 gene correlates with soft tissue neoplasms (Oncogene 20: 6653-9 (2001)). Abnormal expression of EWSR1 protein may correlate with abnormal fibroblast growth factor receptor signaling pathway associated with Ewing's sarcoma (Oncogene 19: 4298-301 (2000)). Translocation of the EWSR1 gene correlates with neuroblastoma tumors associated with nose neoplasms (PNAS 93: 1038-43 (1996)). Translocation of the EWSR1 gene correlates with small cell carcinoma (Proc Natl Acad Sci USA 92: 1028-32 (1995)). Translocation of the EWSR1 gene correlates with small cell carcinoma (Proc Natl Acad Sci USA 92: 1028-32 (1995)). Translocation of the EWSR1 gene correlates with small cell carcinoma (PNAS 92: 1028-32 (1995)). Translocation of the EWSR1 gene correlates with Ewing's sarcoma associated with bone neoplasms (Biochem Biophys Res Commun 293: 61-71 (2002)). Translocation of the EWSR1 gene correlates with clear cell sarcoma (Int J Cancer 99: 560-7 (2002)). Translocation of the EWSR1 gene correlates with neuroblastoma tumors associated with nose neoplasms (Proc Natl Acad Sci USA 93: 1038-43 (1996)). Translocation of the EWSR1 gene correlates with neuroblastoma (Proc Natl Acad Sci USA 93: 1038-43 (1996)). Translocation of the EWSR1 gene correlates with clear cell sarcoma (Oncogene 20: 6653-9 (2001)). Decreased expression of EWSR1 protein may prevent increased cell proliferation associated with Ewing's sarcoma (J Clin Invest 99: 239-47 (1997)). Translocation of the EWSR1 gene may cause melanoma tumors associated with soft tissue neoplasms (Oncogene 10: 1749-56 (1995)). Translocation of the EWSR1 gene correlates with Ewing's sarcoma (Oncogene 19: 3799-804 (2000)). Translocation of the EWSR1 gene may cause abnormal regulation of transcription associated with bone neoplasms (Cancer Res 60: 1536-40 (2000)). Translocation of the EWSR1 gene correlates with Ewing's sarcoma (Oncogene 10: 1229-34 (1995)). Translocation of the EWSR1 gene may cause increased telomere maintenance via telomerase associated with Ewing's sarcoma (Cancer Res 63: 8338-44 (2003)). (PhosphoSite®, Cell Signaling Technology (Danvers, Mass.), Human PSD™, Biobase Corporation, (Beverly, Mass.)).

FASN (P49327), phosphorylated at Y222, is among the proteins listed in this patent. FASN, Fatty acid synthase, multifunctional enzyme that synthesizes fatty acids from dietary proteins and carbohydrates, increased expression is associated with various cancers and inhibition may be therapeutic for breast and prostate cancer. This protein has potential diagnostic and/or therapeutic implications based on the following findings. Induced inhibition of the fatty-acid synthase activity of FASN may prevent increased fatty acid biosynthetic process associated with ovarian neoplasms (Cancer Res 56: 1189-93 (1996)). Induced inhibition of FASN protein may prevent increased cell proliferation associated with breast neoplasms (Cancer Res 60: 213-8 (2000)). Induced inhibition of the fatty-acid synthase activity of FASN may prevent increased cell proliferation associated with breast neoplasms (PNAS 91: 6379-83 (1994)). Increased expression of FASN protein correlates with increased occurrence of more severe form of breast neoplasms (Cancer 77: 474-82 (1996)). Increased expression of FASN in serum correlates with breast neoplasms (Cancer Lett 167: 99-104 (2001)). Increased expression of FASN protein correlates with increased occurrence of invasive form of prostatic neoplasms (Int J Cancer 98: 19-22 (2002)). Induced inhibition of FASN protein may cause increased apoptosis associated with breast neoplasms (Cancer Res 56: 2745-7 (1996)). Induced inhibition of the fatty-acid synthase activity of FASN may prevent increased cell proliferation associated with breast neoplasms (Proc Natl Acad Sci USA 91: 6379-83 (1994)). Increased expression of FASN mRNA may correlate with breast neoplasms (Cancer Lett 149: 43-51 (2000)). Induced inhibition of the fatty-acid synthase activity of FASN may prevent increased cell proliferation associated with breast neoplasms (Proc Natl Acad Sci USA 91: 6379-83 (1994)). (PhosphoSite®, Cell Signaling Technology (Danvers, Mass.), Human PSD™, Biobase Corporation, (Beverly, Mass.)).

FGFR3 (P22607), phosphorylated at Y552, Y577, Y647, Y648, is among the proteins listed in this patent. FGFR3, Fibroblast growth factor receptor 3, inhibits bone formation, involved in cell proliferation, upregulated in urinary tract carcinoma; mutations in corresponding gene cause achondroplasia, thanatophoric dwarfism, skeletal dysplasia and multiple neoplasms. This protein has potential diagnostic and/or therapeutic implications based on the following findings. FGFR3 map position correlates with cherubism (Am J Hum Genet 65: 151-7 (1999)). Missense mutation in the protein kinase domain of FGFR3 causes increased severity of skeleton defects associated with thanatophoric dysplasia (Mol. Cell. Biol 16: 4081-7 (1996)). Missense mutation in the FGFR3 gene causes craniosynostoses (Lancet 349: 1059-62 (1997)). Increased protein dimerization activity of FGFR3 may cause abnormal chondrocytes differentiation associated with thanatophoric dysplasia (Hum Mol Genet 6: 1899-906 (1997)). Increased protein-tyrosine kinase activity of FGFR3 may cause increased cell cycle arrest associated with thanatophoric dysplasia (Nature 386: 288-92 (1997)). Increased transmembrane receptor protein tyrosine kinase activity of FGFR3 causes increased severity of skeleton defects associated with thanatophoric dysplasia (Mol Cell Biol 16: 4081-7 (1996)). Increased fibroblast growth factor receptor activity of FGFR3 causes abnormal fibroblast growth factor receptor signaling pathway associated with achondroplasia (EMBO J. 15: 520-7 (1996)). Mutation in the FGFR3 gene causes abnormal JAK-STAT cascade associated with thanatophoric dysplasia (JBC 273: 13007-14 (1998)). Missense mutation in the FGFR3 gene causes achondroplasia associated with acanthosis nigricans (Am J Hum Genet 64: 722-31 (1999)). Mutation in the FGFR3 gene causes decreased calcium-mediated signaling associated with thanatophoric dysplasia (Hum Mol Genet 6: 681-8 (1997)). Missense mutation in the FGFR3 gene causes acanthosis nigricans associated with achondroplasia (Am J Hum Genet 64: 722-31 (1999)). Increased fibroblast growth factor receptor activity of FGFR3 may cause abnormal MAPKKK cascade associated with multiple myeloma (Blood 97: 729-736 (2001)). Mutation in the Immunoglobulin domain of FGFR3 may cause carcinoma tumors associated with colorectal neoplasms (Cancer Res 61: 3541-3 (2001)). Missense mutation in the FGFR3 gene may cause abnormal fibroblast growth factor receptor signaling pathway associated with achondroplasia (J Clin Invest 104: 1517-25 (1999)). Increased expression of FGFR3 protein correlates with genetic translocation associated with multiple myeloma (Blood 100: 1417-24 (2002)). Abnormal phosphorylation of FGFR3 may cause abnormal skeletal development associated with achondroplasia (J Clin Invest 104: 1517-25 (1999)). Abnormal glycosylation of FGFR3 may cause abnormal tyrosine phosphorylation of Stat1 protein associated with thanatophoric dysplasia (JBC 278: 17344-9 (2003)). Increased stability of FGFR3 may cause thanatophoric dysplasia associated with fetal diseases (Hum Mol Genet 6: 1899-906 (1997)). Increased expression of FGFR3 in cartilage may cause thanatophoric dysplasia associated with fetal diseases (Hum Mol Genet 6: 1899-906 (1997)). Increased expression of FGFR3 mutant protein correlates with genetic translocation associated with multiple myeloma (Blood 92: 3025-34 (1998)). Increased fibroblast growth factor receptor activity of FGFR3 causes abnormal fibroblast growth factor receptor signaling pathway associated with achondroplasia (EMBO 15: 520-7 (1996)). Abnormal expression of FGFR3 mRNA correlates with genetic translocation associated with multiple myeloma (Cancer Res 60: 4058-61 (2000)). Missense mutation in the protein kinase domain of FGFR3 causes increased severity of skeleton defects associated with thanatophoric dysplasia (Mol. Cell. Biol. 16: 4081-7 (1996)). Increased fibroblast growth factor receptor activity of FGFR3 may cause defective bone development associated with achondroplasia (EMBO J 15: 520-7 (1996)). Increased transmembrane receptor protein tyrosine kinase activity of FGFR3 causes increased severity of skeleton defects associated with thanatophoric dysplasia (Mol Cell Biol. 16: 4081-7 (1996)). Translocation of the FGFR3 gene correlates with multiple myeloma (Cancer Res 58: 5640-5 (1998)). Increased expression of FGFR3 mRNA may cause abnormal MAPKKK cascade associated with multiple myeloma (Blood 97: 729-736 (2001)). Mutation in the FGFR3 gene causes abnormal fibroblast growth factor receptor signaling pathway associated with thanatophoric dysplasia (Hum Mol Genet 6: 681-8 (1997)). Point mutation in the FGFR3 gene causes achondroplasia (Cell 78: 335-42 (1994)). Increased transmembrane receptor protein tyrosine kinase activity of FGFR3 causes increased severity of skeleton defects associated with thanatophoric dysplasia (Mol. Cell Biol 16: 4081-7 (1996)). Mutation in the FGFR3 gene causes abnormal JAK-STAT cascade associated with thanatophoric dysplasia (J Biol Chem 273: 13007-14 (1998)). Mutation in the FGFR3 gene causes increased induction of apoptosis associated with thanatophoric dysplasia (JBC 273: 13007-14 (1998)). Increased fibroblast growth factor receptor activity of FGFR3 may cause defective cartilage development associated with achondroplasia (EMBO 15: 520-7 (1996)). Increased expression of FGFR3 mRNA correlates with genetic translocation associated with multiple myeloma (Blood 90: 4062-70 (1997)). Increased fibroblast growth factor receptor activity of FGFR3 may cause thanatophoric dysplasia associated with fetal diseases (Hum Mol Genet 6: 1899-906 (1997)). Increased expression of FGFR3 mutant protein may cause increased cell proliferation associated with multiple myeloma (Blood 95: 992-8 (2000)). Increased protein dimerization activity of FGFR3 may cause thanatophoric dysplasia associated with fetal diseases (Hum Mol Genet 6: 1899-906 (1997)). Single nucleotide polymorphism in the FGFR3 gene causes achondroplasia (Am J Hum Genet 56: 368-73 (1995)). Missense mutation in the FGFR3 gene may cause abnormal skeletal development associated with achondroplasia (J Clin Invest 104: 1517-25 (1999)). Mutation in the FGFR3 gene causes abnormal chondrocytes differentiation associated with thanatophoric dysplasia (JBC 273: 13007-14 (1998)). Missense mutation in the FGFR3 gene causes achondroplasia (Cell 78: 335-42 (1994)). Mutation in the FGFR3 gene causes abnormal chondrocytes differentiation associated with thanatophoric dysplasia (J Biol Chem 273: 13007-14 (1998)). Missense mutation in the FGFR3 gene causes non-familial form of achondroplasia (Am J Hum Genet 63: 711-6 (1998)). Missense mutation in the FGFR3 gene may cause transitional cell carcinoma associated with bladder neoplasms (Oncogene 20: 686-91 (2001)). Mutation in the FGFR3 gene correlates with carcinoma tumors associated with bladder neoplasms (Oncogene 20: 5059-61 (2001)). Missense mutation in the FGFR3 gene may correlate with acrocephalosyndactylia (Hum Mol Genet 6: 1369-73 (1997)). Mutation in the FGFR3 gene causes achondroplasia (Nature 371: 252-4 (1994)). Point mutation in the FGFR3 gene causes craniosynostoses (Lancet 349: 1059-62 (1997)). Increased phosphorylation of FGFR3 may cause abnormal cell surface receptor linked signal transduction associated with multiple myeloma (Oncogene 20: 3553-62 (2001)). Increased transmembrane receptor protein tyrosine kinase activity of FGFR3 causes developmental bone diseases (Am J Hum Genet 67: 1411-21 (2000)). Missense mutation in the protein kinase domain of FGFR3 causes increased severity of skeleton defects associated with thanatophoric dysplasia (MCB 16: 4081-7 (1996)). Mutation in the FGFR3 gene causes decreased chondrocytes survival associated with thanatophoric dysplasia (JBC 273: 13007-14 (1998)). Abnormal glycosylation of FGFR3 may cause abnormal tyrosine phosphorylation of Stat1 protein associated with thanatophoric dysplasia (J Biol Chem 278: 17344-9 (2003)). Increased fibroblast growth factor receptor activity of FGFR3 may cause defective cartilage development associated with achondroplasia (EMBO J 15: 520-7 (1996)). Mutation in the FGFR3 gene correlates with early stage or low grade form of bladder neoplasms (Cancer Res 61: 1265-8 (2001)). Missense mutation in the FGFR3 gene causes acrocephalosyndactylia (Am J Hum Genet 62: 1370-80 (1998)). Alternative form of FGFR3 mRNA may cause carcinoma tumors associated with colorectal neoplasms (Cancer Res 60: 4049-52 (2000)). Point mutation in the FGFR3 gene causes defective skeleton development associated with craniosynostoses (Am J Hum Genet 60: 555-64 (1997)). Missense mutation in the protein kinase domain of FGFR3 causes developmental bone diseases (Am J Hum Genet 67: 1411-21 (2000)). Translocation of the FGFR3 gene correlates with plasmacytic leukemia (Cancer Res 58: 5640-5 (1998)). Translocation of the FGFR3 locus correlates with multiple myeloma (Blood 90: 4062-70 (1997)). Increased fibroblast growth factor receptor activity of FGFR3 may cause defective bone development associated with achondroplasia (EMBO 15: 520-7 (1996)). Abnormal phosphorylation of FGFR3 may cause abnormal fibroblast growth factor receptor signaling pathway associated with achondroplasia (J Clin Invest 104: 1517-25 (1999)). Increased fibroblast growth factor receptor activity of FGFR3 may cause abnormal chondrocytes differentiation associated with thanatophoric dysplasia (Hum Mol Genet 6: 1899-906 (1997)). Increased transmembrane receptor protein tyrosine kinase activity of FGFR3 causes achondroplasia associated with acanthosis nigricans (Am J Hum Genet 64: 722-31 (1999)). Mutation in the FGFR3 gene correlates with carcinoma tumors associated with bladder neoplasms (Oncogene 20: 4416-8 (2001)). Increased protein-tyrosine kinase activity of FGFR3 may cause increased STAT protein nuclear translocation associated with thanatophoric dysplasia (Nature 386: 288-92 (1997)). Missense mutation in the protein kinase domain of FGFR3 causes increased severity of skeleton defects associated with thanatophoric dysplasia (Mol Cell Biol 16: 4081-7 (1996)). Increased transmembrane receptor protein tyrosine kinase activity of FGFR3 causes acanthosis nigricans associated with achondroplasia (Am J Hum Genet 64: 722-31 (1999)). Missense mutation in the FGFR3 gene causes thanatophoric dysplasia (Hum Mol Genet 5: 509-12 (1996)). Increased fibroblast growth factor receptor activity of FGFR3 may cause defective bone development associated with achondroplasia (EMBO J. 15: 520-7 (1996)). Increased expression of FGFR3 mutant protein may cause increased anti-apoptosis associated with multiple myeloma (Blood 95: 992-8 (2000)). Increased expression of FGFR3 in cartilage may cause abnormal chondrocytes differentiation associated with thanatophoric dysplasia (Hum Mol Genet 6: 1899-906 (1997)). Mutation in the FGFR3 gene causes increased induction of apoptosis associated with thanatophoric dysplasia (J Biol Chem 273: 13007-14 (1998)). Mutation in the FGFR3 gene causes decreased fibroblast growth factor receptor signaling pathway associated with achondroplasia (Hum Mol Genet 6: 681-8 (1997)). Increased fibroblast growth factor receptor activity of FGFR3 may cause defective cartilage development associated with achondroplasia (EMBO J. 15: 520-7 (1996)). Increased fibroblast growth factor receptor activity of FGFR3 causes abnormal fibroblast growth factor receptor signaling pathway associated with achondroplasia (EMBO J. 15: 520-7 (1996)). Increased stability of FGFR3 may cause abnormal chondrocytes differentiation associated with thanatophoric dysplasia (Hum Mol Genet 6: 1899-906 (1997)). Increased nucleus localization of FGFR3 may cause thanatophoric dysplasia associated with fetal diseases (Hum Mol Genet 6: 1899-906 (1997)). Missense mutation in the FGFR3 gene causes defective skeleton development associated with craniosynostoses (Am J Hum Genet 60: 555-6 (1997)). Increased transmembrane receptor protein tyrosine kinase activity of FGFR3 causes increased severity of skeleton defects associated with thanatophoric dysplasia (MCB 16: 4081-7 (1996)). Increased transmembrane receptor protein tyrosine kinase activity of FGFR3 causes increased severity of skeleton defects associated with thanatophoric dysplasia (Mol. Cell. Biol. 16: 4081-7 (1996)). Increased expression of FGFR3 mRNA correlates with genetic translocation associated with multiple myeloma (Blood 99: 1745-57 (2002)). Increased expression of FGFR3 mRNA may cause drug-resistant form of multiple myeloma (Blood 100: 3819-21 (2002)). Mutation in the FGFR3 gene causes decreased calcium-mediated signaling associated with achondroplasia (Hum Mol Genet 6: 681-8 (1997)). Abnormal mRNA splicing of FGFR3 may cause carcinoma tumors associated with colorectal neoplasms (Cancer Res 60: 4049-52 (2000)). Increased protein-tyrosine kinase activity of FGFR3 may cause abnormal regulation of ossification associated with thanatophoric dysplasia (Nature 386: 288-92 (1997)). Missense mutation in the FGFR3 gene may cause transitional cell carcinoma (Oncogene 20: 686-91 (2001)). Increased protein-tyrosine kinase activity of FGFR3 may cause increased tyrosine phosphorylation of Stat1 protein associated with thanatophoric dysplasia (Nature 386: 288-92 (1997)). Mutation in the FGFR3 gene causes decreased chondrocytes survival associated with thanatophoric dysplasia (J Biol Chem 273: 13007-14 (1998)). Increased fibroblast growth factor receptor activity of FGFR3 may cause abnormal cell surface receptor linked signal transduction associated with multiple myeloma (Oncogene 20: 3553-62 (2001)). Increased nucleus localization of FGFR3 may cause abnormal chondrocytes differentiation associated with thanatophoric dysplasia (Hum Mol Genet 6: 1899-906 (1997)). Missense mutation in the protein kinase domain of FGFR3 causes increased severity of skeleton defects associated with thanatophoric dysplasia (Mol Cell Biol. 16: 4081-7 (1996)). (PhosphoSite®, Cell Signaling Technology (Danvers, Mass.), Human PSD™, Biobase Corporation, (Beverly, Mass.)).

Fgr (P09769), phosphorylated at Y208, Y209, is among the proteins listed in this patent. Fgr, Gardner-Rasheed feline sarcoma viral oncogene homolog, acts in integrin signaling, neutrophil degranulation, and antiapoptosis, may be upregulated in Epstein-Barr-infected cells; gene amplification correlates with hormone-resistance in prostate cancer. This protein has potential diagnostic and/or therapeutic implications based on the following findings. Induced inhibition of FGR protein may prevent increased cell proliferation associated with acute B-cell leukemia (Nat Genet 36: 453-61 (2004)). Alternative form of FGR mRNA correlates with Epstein-Barr virus infections (Mol Cell Biol 11: 1500-7 (1991)). Alternative form of FGR mRNA correlates with Epstein-Barr virus infections (Mol. Cell Biol 11: 1500-7 (1991)). Increased expression of FGR mRNA may correlate with Epstein-Barr virus infections (Nature 319: 238-40 (1986)). Alternative form of FGR mRNA correlates with Epstein-Barr virus infections (Mol. Cell. Biol. 11: 1500-7 (1991)). Alternative form of FGR mRNA correlates with Epstein-Barr virus infections (Mol Cell Biol. 11: 1500-7 (1991)). Alternative form of FGR mRNA correlates with Epstein-Barr virus infections (MCB 11: 1500-7 (1991)). (PhosphoSite®, Cell Signaling Technology (Danvers, Mass.), Human PSD™, Biobase Corporation, (Beverly, Mass.)).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. As used herein, unless specifically indicated otherwise, the word “or” is used in the “inclusive” sense of “and/or” and not the “exclusive” sense of “either/or.”

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables which are inherently discrete, and can be 0.0, 0.1, 0.01, 0.001, or any other real value for variables which are inherently continuous.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

“Antibody” or “antibodies” refers to all classes of immunoglobulins, including

IgG, IgM, IgA, IgD, and IgE, including whole antibodies and any antigen biding fragment thereof (e.g., F_{ab}) or single chains thereof, including chimeric, polyclonal, and monoclonal antibodies. Antibodies are antigen-specific protein molecules produced by lymphocytes of the B cell lineage. Following antigenic stimulation, B cells that have surface immunoglobulin receptors that bind the antigen clonally expand, and the binding affinity for the antigen increases through a process called affinity maturation. The B cells further differentiate into plasma cells, which secrete large quantities of antibodies in to the serum. While the physiological role of antibodies is to protect the host animal by specifically binding and eliminating microbes and microbial pathogens from the body, large amounts of antibodies are also induced by intentional immunization to produce specific antibodies that are used extensively in many biomedical and therapeutic applications.

Antibody molecules are shaped somewhat like the letter “Y”, and consist of 4 protein chains, two heavy (H) and two light (L) chains. Antibodies possess two distinct and spatially separate functional features. The ends of each of the two arms of the “Y” contain the variable regions (variable heavy (V(H)) and variable light (V(L)) regions), which form two identical antigen-binding sites. The variable regions undergo a process of “affinity maturation” during the immune response, leading to a rapid divergence of amino acids within these variable regions. The other end of the antibody molecule, the stem of the “Y”, contains only the two heavy constant (CH) regions, interacts with effector cells to determine the effector functions of the antibody. There are five different CH region genes that encode the five different classes of immunoglobulins: IgM, IgD, IgG, IgA and IgE. These constant regions, by interacting with different effector cells and molecules, determine the immunoglobulin molecule's biological function and biological response.

Each V(H) and V(L) region contains three subregions called complementarity determining regions. These include CDR1-3 of the V(H) domain and CDR1-3 of the V(L) domain. These six CDRs generally form the antigen binding surface, and include those residues that hypermutate during the affinity maturation phase of the immune response. The CDR3 of the V(H) domain seems to play a dominant role in generating diversity of both the B cell antigen receptor (BCR) and the T cell antigen receptor systems (Xu et al., Immunity 13:37-45 (2000)).

The term “antibody” or “antibodies” refers to all classes of polyclonal or monoclonal immunoglobulins, including IgG, IgM, IgA, IgD, and IgE, including whole antibodies and any antigen binding fragment thereof. This includes any combination of immunoglobulin domains or chains that contains a variable region (V(H) or V(L)) that retains the ability to bind the immunogen. Such fragments include F(ab)₂ fragments (V(H)—C(H1), V(L)-C(L))₂; monovalent Fab fragments (V(H)—C(H1), V(L)-C(L)); Fv fragment (V(H)-V(L); single-chain Fv fragments (Kobayashi et al., Steroids July; 67(8):733-42 (2002).

Monoclonal antibodies refer to clonal antibodies produced from fusions between immunized murine, rabbit, human, or other vertebrate species, and produced by classical fusion technology Kohler G, Milstein C. Continuous cultures of fused cells secreting antibody of predefined specificity. Nature 1975 Aug. 7; 256(5517):495-7 or by alternative methods which may isolate clones of immunoglobulin secreting cells from transformed plasma cells.

When used with respect to an antibody's binding to one phospho-form of a sequence, the expression “does not bind” means that a phospho-specific antibody either does not apparently bind to the non-phospho form of the antigen as ascertained in commonly used experimental detection systems (Western blotting, IHC, Immunofluorescence, etc.). One of skill in the art will appreciate that the expression may be applicable in those instances when (1) a phospho-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the phosphorylated residue is an immunodominant feature of the reaction. In cases such as these, there is an apparent difference in affinities for the two sequences. Dilutional analyses of such antibodies indicates that the antibodies apparent affinity for the phosphorylated form is at least 10-100 fold higher than for the non-phosphorylated form; or where (3) the phospho-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

“Target signaling protein/polypeptide” means any protein (or polypeptide derived therefrom) enumerated in Column A of Table 1/FIG. 2, which is disclosed herein as being phosphorylated in one or more cell line(s). Target signaling protein(s)/polypeptide(s) may be tyrosine kinases, such as TTN or BCR, or serine/threonine kinases, or direct substrates of such kinases, or may be indirect substrates downstream of such kinases in signaling pathways. Target signaling protein/polypeptide where elucidated in leukemia cell lines, however one of skill in the art will appreciate that a target signaling protein/polypeptide may also be phosphorylated in other cell lines (non-leukemic) harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with peptide and polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

A. Identification of Phosphorylation Sites. The target signaling protein/polypeptide phosphorylation sites disclosed herein and listed in Table 1/FIG. 2 were discovered by employing the modified peptide isolation and characterization techniques described in U.S. Pat. No. 7,198,896 using cellular extracts from the following human cancer cell lines, tissues and patient samples: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F-jak2, Baf3/E255K, Baf3/H396P, Baf3/Jak2(IL-3 dep), Baf3/M351T, Baf3/T315I, Baf3/TpoR, Baf3/TpoR-Y98F, Baf3/Tyk2, Baf3/V617F-jak2 (IL-3), Baf3/Y253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A (Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-ly8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, hl144b, hl152b, lung tumor T26, lung tumor T57, normal human lung, pancreatic xenograft, patient 1, rat brain, sw480. The isolation and identification of phosphopeptides from these cell lines, using an immobilized general phosphotyrosine-specific antibody, or an antibody recognizing the phosphorylated motif PXpSP is described in detail in Example 1 below. In addition to the protein phosphorylation sites (tyrosine) described herein, many known phosphorylation sites were also identified (not described herein). The immunoaffinity/mass spectrometric technique described in the '896 Patent (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g., Sequest) may be utilized to substantially match the spectra obtained for the isolated, modified peptide during the characterization of step (d) with the spectra for a known peptide sequence. A quantification step employing, e.g., SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat. #9411 (p-Tyr-100)) was used in the immunoaffinity step to isolate the widest possible number of phospho-tyrosine containing peptides from the cell extracts.

Extracts from the following human cancer cell lines, tissues and patient samples were employed: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F-jak2, Baf3/E255K, Baf3/H396P, Baf3/Jak2(IL-3 dep), Baf3/M351T, Baf3/T315I, Baf3/TpoR, Baf3/TpoR-Y98F, Baf3/Tyk2, Baf3N617F-jak2 (IL-3), Baf3/Y253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A (Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-ly8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, hl144b, hl152b, lung tumor T26, lung tumor T57, normal human lung, pancreatic xenograft, patient 1, rat brain and sw480.

As described in more detail in the Examples, lysates were prepared from these cells and digested with trypsin after treatment with DTT and iodoacetamide to redue and alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in MOPS IP buffer and treated with phosphotyrosine (P-Tyr-100, CST #9411) immobilized on protein G-Sepharose. Immunoaffinity-purified peptides were eluted with 0.1% TFA and a portion of this fraction was concentrated with Stage or Zip tips and analyzed by LC-MS/MS, using either a LCQ or ThermoFinnigan LTQ ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed the tyrosine phosphorylation sites in signaling pathways affected by kinase activation or active in leukemia cells. The identified phosphorylation sites and their parent proteins are enumerated in Table 1/FIG. 2. The tyrosine at which phosphorylation occurs is provided in Column D, and the peptide sequence encompassing the phosphorylatable tyrosine residue at the site is provided in Column E. If a phosphorylated tyrosine was found in mouse, the orthologous site in human was identified using either Homologene or BLAST at NCBI; the sequence reported in column E is the phosphorylation site flanked by 7 amino acids on each side. FIG. 2 also shows the particular type of leukemic disease (see Column G) and cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, as described below. These new reagents will prove highly useful in, e.g., studying the signaling pathways and events underlying the progression of leukemias and the identification of new biomarkers and targets for diagnosis and treatment of such diseases in a mammal.

The methods of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

B. Antibodies and Cell Lines. Isolated phosphorylation site-specific antibodies that specifically bind a target signaling protein/polypeptide disclosed in Column A of Table 1 only when phosphorylated (or only when not phosphorylated) at the corresponding amino acid and phosphorylation site listed in Columns D and E of Table 1/FIG. 2 may be produced by standard antibody production methods, such as anti-peptide antibody methods, using the phosphorylation site sequence information provided in Column E of Table 1. The ANK1 adaptor/scaffold protein phosphorylation site (tyrosine 1258) (see Row [#] 16 of Table 1/FIG. 2) is presently disclosed. Thus, an antibody that specifically binds this novel ANK1 adaptor/scaffold site can now be produced, e.g. by immunizing an animal with a peptide antigen comprising all or part of the amino acid sequence encompassing the respective phosphorylated residue (e.g., a peptide antigen comprising the sequence set forth in Row [#] 16, Column E, of Table 1, SEQ ID NO: 15, respectively) (which encompasses the phosphorylated tyrosine at position 1258 in ANK1, to produce an antibody that only binds ANK1 adaptor/scaffold when phosphorylated at that site.

Polyclonal antibodies of the invention may be produced according to standard techniques by immunizing a suitable animal (e.g., rabbit, goat, etc.) with a peptide antigen corresponding to the phosphorylation site of interest (i.e., a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel Crkl adaptor/scaffold phosphorylation site disclosed herein (SEQ ID NO: 37=IHyLDTTTLIEPAPR, encompassing phosphorylated tyrosine 92 (see Row 38 of Table 1)) may be employed to produce antibodies that only bind Crkl when phosphorylated at Tyr 92. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., Antibodies: A Laboratory Manual, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by lowercase “y”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265: 495-97 (1975); Kohler and Milstein, Eur. J. Immunol. 6: 511 (1976); see also, Current Protocols in Molecular Biology, Ausubel et al. Eds (1989); Harlow E, D Lane. Antibodies, A Laboratory Manual. CSHP 1988. Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal F_{ab} fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferable for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

Additional methods available include vaccination of the animal with DNA or virus encoding the protein of interest (Bates et al., Biotechniques. February; 40(2):199-208 (2006)).

An epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the CGN tyrosine 55 phosphorylation site sequence disclosed in Row 35, Column E of Table 1), and antibodies of the invention thus specifically bind a target Signal Protein/Polypeptide comprising such epitopic sequence. Epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 or U.S. Pat. No. 4,816,567. The antibodies may also be chemically constructed by specific antibodies made according to the method disclosed in U.S. Pat. No. 4,676,980.

The invention also provides immortalized cell lines that produce an antibody of the invention. For example, hybridoma clones, constructed as described above, that produce monoclonal antibodies to the protein phosphorylation sites disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g., Czernik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given target Signal Protein/Polypepetide. The antibodies may also be tested by Western blotting against cell preparations containing the signaling protein, e.g. cell lines over-expressing the target protein, to confirm reactivity with the desired phosphorylated epitope/target.

In an exemplary embodiment, phage display libraries containing more than 10¹⁰ phage clones are used for high-throughput production of monoclonal antibodies that target post-translational modification sites (e.g., phosphorylation sites) and, for validation and quality control, high-throughput immunohistochemistry is utilized to screen the efficacy of these antibodies. Western blots, protein microarrays and flow cytometry can also be used in high-throughput screening of phosphorylation site-specific polyclonal or monoclonal antibodies of the present invention. See, e.g., Blow N., Nature, 447: 741-743 (2007).

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the target signaling protein/polypeptide epitope for which the antibody of the invention is specific.

In certain cases, polyclonal antisera may exhibit some undesirable general cross-reactivity to phosphotyrosine or phosphoserine itself, which may be removed by further purification of antisera, e.g., over a phosphotyramine column. Antibodies of the invention specifically bind their target protein (i.e., a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to evaluate phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a target Signal Protein/Polypepetide enumerated in Table 1), washed and labeled with a fluorescent-labeled secondary antibody. Additional fluorochrome-conjugated marker antibodies (e.g., CD45, CD34) may also be added at this time to aid in the subsequent identification of specific hematopoietic cell types. The cells would then be analyzed on a flow cytometer (e.g., a Beckman Coulter FC500) according to the specific protocols of the instrument used.

Antibodies of the invention may also be advantageously conjugated to fluorescent dyes (e.g., Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a target signaling protein/polypeptide only when phosphorylated at a disclosed site, but are not limited only to binding the human species, per se. The invention includes antibodies that also bind conserved and highly homologous or identical phosphorylation sites in respective target signaling protein/polypeptide from other species (e.g., mouse, rat, monkey, yeast), in addition to binding the human phosphorylation site. Highly homologous or identical sites conserved in other species can readily be identified by standard sequence comparisons, such as using BLAST, with the human target signaling protein/polypeptide phosphorylation sites disclosed herein.

C. Heavy-Isotope Labeled Peptides (AQUA Peptides). The phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, Gerber et al., Proc. Natl. Acad. Sci. U.S.A. 100: 6940-5 (2003).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis (See Gerber et al., supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g., trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g., 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g., trypsin, hepsin), metallo proteases (e.g., PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. A workable range is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e., to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: the mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are suitable labels. Pairs of peptide internal standards that incorporate a different isotope label may also be prepared. Amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts may be employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g., by HPLC, reverse-phase HPLC, capillary electrophoresis, ion exchange chromatography, etc.) to isolate the labeled internal standard and its corresponding target peptide from other peptides in the sample. Microcapillary LC is a method contemplated.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the phosphorylation sites disclosed herein. Peptide standards for a given phosphorylation site (e.g., the tyrosine 644 in CD93—see Row 48 of Table 1) may be produced for both the phosphorylated and non-phosphorylated forms of the site (e.g., see FASN site sequence in Column E, Row 195 of Table 1 (SEQ ID NO: 196) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In an embodiment, an AQUA peptide of the invention comprises a phosphorylation site sequence disclosed herein in Table 1/FIG. 2. For example, an AQUA peptide of the invention for detection/quantification of BAG4 Apoptosis protein when phosphorylated at tyrosine Y102 may comprise the sequence SGYGPSDGPSyGR (y=phosphotyrosine), which comprises phosphorylatable tyrosine 102 (see Row 54, Column E; (SEQ ID NO: 53)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the phosphorylation sites disclosed in Table 1/FIG. 2 (see Column E) and/or their corresponding parent proteins/polypeptides (see Column A). A phosphopeptide sequence comprising any of the phosphorylation sequences listed in Table 1 may be considered an AQUA peptide of the invention. For example, an AQUA peptide comprising the sequence EMVSQYLyTSK (SEQ ID NO: 38) (where y may be either phosphotyrosine or tyrosine, and where V=labeled valine (e.g., ¹⁴C)) is provided for the quantification of phosphorylated (or non-phosphorylated) diaphanous1 (Tyr130) in a biological sample (see Row 39 of Table 1, tyrosine 130 being the phosphorylatable residue within the site). It will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of AQUA peptides may be carried out as described above (see Gygi et al., Gerber et al., supra.).

Certain subsets of AQUA peptides provided by the invention are described above (corresponding to particular protein types/groups in Table 1, for example, tyrosine protein kinases or adaptor/scaffold proteins). Example 4 is provided to further illustrate the construction and use, by standard methods described above, of exemplary AQUA peptides provided by the invention. For example, the above-described AQUA peptides corresponding to both the phosphorylated and non-phosphorylated forms of the disclosed claspin cell cycle regulation protein tyrosine 887 phosphorylation site (see Row 80 of Table 1/FIG. 2) may be used to quantify the amount of phosphorylated claspin (Tyr 887) in a biological sample, e.g., a tumor cell sample (or a sample before or after treatment with a test drug).

AQUA peptides of the invention may also be employed within a kit that comprises one or multiple AQUA peptide(s) provided herein (for the quantification of a target signaling protein/polypeptide disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be useful in the further study of signal transduction anomalies associated with diseases such as for example cancer, including leukemias, and in identifying diagnostic/bio-markers of these diseases, new potential drug targets, and/or in monitoring the effects of test compounds on target Signaling Proteins/Polypeptides and pathways.

D. Immunoassay Formats. Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022; U.S. Pat. No. 4,659,678; U.S. Pat. No. 4,376,110. Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target signaling protein/polypeptide is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a target signaling protein/polypeptide in patients before, during, and after treatment with a drug targeted at inhibiting phosphorylation of such a protein at the phosphorylation site disclosed herein. For example, bone marrow cells or peripheral blood cells from patients may be analyzed by flow cytometry for target signaling protein/polypeptide phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% paraformaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g., a Beckman Coulter EPICS-XL) according to the specific protocols of the instrument used. Such an analysis would identify the presence of activated target Signaling Protein(s)/Polypeptide(s) in the malignant cells and reveal the drug response on the targeted protein.

Alternatively, antibodies of the invention may be employed in immunohistochemical (IHC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody array formats, such as reversed-phase array applications (see, e.g., Paweletz et al., Oncogene 20(16): 1981-89 (2001)). Accordingly, in another embodiment, the invention provides a method for the multiplex detection of phosphorylation in a biological sample, the method comprising utilizing two or more antibodies or AQUA peptides of the invention to detect the presence of two or more phosphorylated proteins enumerated in Column A of Table 1/FIG. 2. In an embodiment, two to five antibodies or AQUA peptides of the invention are employed in the method. In another embodiment, six to ten antibodies or AQUA peptides of the invention are employed, while in another embodiment eleven to twenty such reagents are employed.

Antibodies and/or AQUA peptides of the invention may also be employed within a kit that comprises at least one phosphorylation site-specific antibody or AQUA peptide of the invention (which binds to or detects a target signaling protein/polypeptide disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the description, numerous specific details are set forth in order to provide a thorough understanding of the present invention. The present invention may be practiced without some or all of these specific details. In other instances, well known process operations have not been described in detail, in order not to unnecessarily obscure the present invention.

The following examples are intended to further illustrate certain embodiments of the invention and are not limiting in nature. Those skilled in the art will recognize, or be able to ascertain, using no more than routine experimentation, numerous equivalents to the specific substances and procedures described herein.

Any suitable materials and/or methods known to those of skill can be utilized in carrying out the present invention. However, materials and methods are described. Materials, reagents and the like to which reference is made in the following description and examples are obtainable from commercial sources, unless otherwise noted.

### Example 1

### Isolation of Phosphotyrosine-Containing Peptides from Extracts of Cancer Cell Lines and Identification of Phosphorylation Sites

IAP isolation techniques were employed to identify phosphotyrosine containing peptides in cell extracts from the following human cancer cell lines, tissues and patient cell lines: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F-jak2, Baf3/E255K, Baf3/H396P, Baf3/Jak2 (IL-3 dep), Baf3/M351T, Baf3/T3151, Baf3/TpoR, Baf3/TpoR-Y98F, Baf3/Tyk2, Baf3N617F-jak2 (IL-3), Baf3/Y253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A (Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-ly8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, hl144b, hl152b, lung tumor T26, lung tumor T57, normal human lung, pancreatic xenograft, patient 1, rat brain and sw480.

Tryptic phosphotyrosine containing peptides were purified and analyzed from extracts of each of the cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK®-trypsin (Worthington® Biochemcial Corporation, Lakewood, N.J.) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak® C₁₈ columns (provided by Waters Corporation, Milford, Mass.) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble material was removed by centrifugation. IAP was performed on each peptide fraction separately. The phosphotyrosine monoclonal antibody P-Tyr-100 (Cell Signaling Technology®, Inc., Danvers, Mass. catalog number 9411) was coupled at 4 mg/ml beads to protein G or protein A agarose (Roche®, Basel, Switzerland), respectively. Immobilized antibody (15 μl, 60 μg) was added as 1:1 slurry in IAP buffer to 1.4 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak® C₁₈ columns (provided by Waters Corporation, Milford, Mass.) by elution with 2 volumes each of 10%, 15%, 20%, 25%, 30%, 35% and 40% acetonitirile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble material was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 40 μl of 0.15% TFA at room temperature for 10 min (eluate 1), followed by a wash of the beads (eluate 2) with 40 μl of 0.15% TFA. Both eluates were combined.

Analysis by LC-MS/MS Mass Spectrometry. 40 μl or more of IAP eluate were purified by 0.2 μl StageTips (Proxeon, Staermosegaardsvej 6, DK-5230 Odense M, Denmark) or ZipTips® (produced by Millipore®, Billerica Mass.). Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit® capillary column (produced by New Objective, Woburn, Mass.) packed with Michrom Magic Bullets® C18 AQ reversed-phase resin (Michrom Bioresources, Auburn Calif.) using a Famos™ autosampler with an inert sample injection valve (Dionex®, Sunnyvale, Calif.). The column was then developed with a 45-min linear gradient of acetonitrile delivered at 200 nl/min (using an Ultimate® pump, Dionex®, Sunnyvale, Calif.), and tandem mass spectra were collected in a data-dependent manner with an LTQ® (produced by Thermo® Finnigan® San, Jose, Calif.), ion trap mass spectrometer essentially as described by Gygi et al., supra.
Database Analysis & Assignments. MS/MS spectra were evaluated using TurboSequest™ in the Sequest (owned by Thermo® Finnigan® San Jose, Calif.) Browser package (v. 27, rev. 12) supplied as part of BioWorks™ 3.0 (Thermo® Finnigan®, San Jose, Calif.). Individual MS/MS spectra were extracted from the raw data file using the Sequest® Browser program CreateDta™ (owned by Thermo® Finnigan® San Jose, Calif.), with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest™ and VuDta™ (owned by Thermo® Finnigan® San Jose, Calif.) programs were not used to further select MS/MS spectra for Sequest® analysis. MS/MS spectra were evaluated with the following TurboSequest™ parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (as released on Aug. 24, 2004 and containing 27, 960 protein sequences). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned. Furthermore, it should be noted that certain peptides were originally isolated in mouse and later normalized to human sequences as shown by Table 1/FIG. 2.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### Example 2

### Production of Phospho-Specific Polyclonal Antibodies for the Detection of Target Signal Protein/Polypepetide Phosphorylation

Polyclonal antibodies that specifically bind a target signal protein/polypeptide only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

A. 14-3-3 zeta (tyrosine 82)

A 17 amino acid phospho-peptide antigen, KQQMAREy*REKIETELR (where y*=phosphotyrosine) that corresponds to the sequence encompassing the tyrosine 82 phosphorylation site in human 14-3-3 zeta adaptor/scaffold protein (see Row 2 of Table 1; SEQ ID NO: 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific 14-3-3 zeta (tyr82) polyclonal antibodies as described in Immunization/Screening below.

B. Crkl (tyrosine 48)

An 18 amino acid phospho-peptide antigen, DSSTCPGDy*VLSVSENSR (where y*=phosphotyrosine) that corresponds to the sequence encompassing the tyrosine 48 phosphorylation site in human Crkl adaptor/scaffold protein (see Row 37 of Table 1 (SEQ ID NO: 36)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific Crkl (tyr 48) polyclonal antibodies as described in Immunization/Screening below.

C. Catalase (tyrosine 84)

A 16 amino acid phospho-peptide antigen, GAGAFGy*FEVTHDITK (where y*=phosphotyrosine) that corresponds to the sequence encompassing the tyrosine 84 phosphorylation site in human catalase apoptosis protein (see Row 59 of Table 1 (SEQ ID NO: 58), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific catalase (tyr 84) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see Antibodies: A Laboratory Manual, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated 14-3-3 zeta, Crkl or catalase), for example, SEM and Jurkat cells, respectively. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. Crkl is not bound when not phosphorylated at tyrosine 48).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### Example 3

### Production of Phospho-Specific Monoclonal Antibodies for the Detection of Target Signal Protein/Polypepetide Phosphorylation

Monoclonal antibodies that specifically bind a target signal protein/polypepetide only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

A. ANXA11 (tyrosine 365)

A 12 amino acid phospho-peptide antigen, DAQELy*AAGENR (where y*=phosphotyrosine) that corresponds to the sequence encompassing the tyrosine 365 phosphorylation site in human ANXA11 calcium binding protein (see Row 62 of Table 1 (SEQ ID NO: 61)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal ANXA11 (tyr 365) antibodies as described in Immunization/Fusion/Screening below.

B. ANXA2 (tyrosine 199)

An 9 amino acid phospho-peptide antigen, DLy*DAGVKR (where y*=phosphotyrosine) that corresponds to the sequence encompassing the tyrosine 199 phosphorylation site in human ANXA2 calcium binding protein (see Row 63 of Table 1 (SEQ ID NO: 62)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal ANXA2 (tyr199) antibodies as described in Immunization/Fusion/Screening below.

C. ANXA5 (tyrosine 256)

A 15 amino acid phospho-peptide antigen, SIPAYLAETLy*YAMK (where y*=phosphotyrosine) that corresponds to the sequence encompassing the tyrosine 256 phosphorylation site in human ANXA5 calcium binding protein (see Row 64 of Table 1 (SEQ ID NO: 63), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal ANXA5 (tyr256) antibodies as described in Immunization/Fusion/Screening below.

Immunization/Fusion/Screening. A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the ANXA11, ANXA2 or ANXA5 phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. ANXA5 phosphorylated at tyrosine 255).

### Example 4

### Production and Use of AQUA Peptides for the Quantification of Target Signal Protein/Polypepetide Phosphorylation

Heavy-isotope labeled peptides (AQUA peptides (internal standards)) for the detection and quantification of a target signal protein/polypepetide only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

A. ANXA11 (tyrosine 365)

An AQUA peptide comprising the sequence, DAQELyAAGENR (y*=phosphotyrosine; sequence incorporating ¹⁴C/¹⁵N-labeled leucine (indicated by bold L), which corresponds to the tyrosine 365 phosphorylation site in human ANXA11 chromatin or DNA binding/repair/replication protein (see Row 62 in Table 1 (SEQ ID NO: 61)), is constructed according to standard synthesis techniques using, e.g., a Rainin/Protein Technologies, Inc., Symphony peptide synthesizer (see Merrifield, supra.) as further described below in Synthesis & MS/MS Signature. The ANXA11 (tyr 365) AQUA peptide is then spiked into a biological sample to quantify the amount of phosphorylated ANXA11 (tyr 365) in the sample, as further described below in Analysis & Quantification.

B. Arp3 (tyrosine 16)

An AQUA peptide comprising the sequence LPACVVDCGTGy*TK (y*=phosphotyrosine; sequence incorporating ¹⁴C/¹⁵N-labeled leucine (indicated by bold L), which corresponds to the tyrosine 16 phosphorylation site in human Arp3 cytoskeletal protein (see Row 105 in Table 1 (SEQ ID NO: 104)), is constructed according to standard synthesis techniques using, e.g., a Rainin/Protein Technologies, Inc., Symphony peptide synthesizer (see Merrifield, supra.) as further described below in Synthesis & MS/MS Signature. The Arp3 (tyr16) AQUA peptide is then spiked into a biological sample to quantify the amount of phosphorylated Arp3 (tyr16) in the sample, as further described below in Analysis & Quantification.

C. ADA (tyrosine 67)

An AQUA peptide comprising the sequence FDy*YMPAIAGCR (y*=phosphotyrosine; sequence incorporating ¹⁴C/¹⁵N-labeled phenylalanine (indicated by bold F), which corresponds to the tyrosine 67 phosphorylation site in human ADA enzyme protein (see Row 146 in Table 1 (SEQ ID NO: 145)), is constructed according to standard synthesis techniques using, e.g., a Rainin/Protein Technologies, Inc., Symphony peptide synthesizer (see Merrifield, supra.) as further described below in Synthesis & MS/MS Signature. The ADA (tyr67) AQUA peptide is then spiked into a biological sample to quantify the amount of phosphorylated ADA (tyr67) in the sample, as further described below in Analysis & Quantification.

D. ASS (tyrosine 133)

An AQUA peptide comprising the sequence, FELSCY*SLAPQIK (y*=phosphotyrosine; sequence incorporating ¹⁴C/¹⁵N-labeled proline (indicated by bold P), which corresponds to the tyrosine 133 phosphorylation site in human ASS enzyme protein (see Row 160 in Table 1 (SEQ ID NO: 159)), is constructed according to standard synthesis techniques using, e.g., a Rainin/Protein Technologies, Inc., Symphony peptide synthesizer (see Merrifield, supra.) as further described below in Synthesis & MS/MS Signature. The ASS (tyr133) AQUA peptide is then spiked into a biological sample to quantify the amount of phosphorylated ASS (tyr133) in the sample, as further described below in Analysis & Quantification.

Synthesis & MS/MS Spectra. Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino)methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e., a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong γ-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

Analysis & Quantification. Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LTQ ion trap or TSQ Quantum triple quadrupole). On the LTQ, parent ions are isolated at 1.6 m/z width, the ion injection time being limited to 100 ms per microscan, with one microscans per peptide, and with an AGC setting of 1×10⁵; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. An isolated phosphorylation site-specific antibody that specifically binds a human signaling protein selected from Column A of Table 1, Rows 248, 239, 244, 262, and 492 only when phosphorylated at the tyrosine listed in corresponding Column D of Table 1, comprised within the phosphorylatable peptide sequence listed in corresponding Column E of Table 1 (SEQ ID NOs: 247, 238, 244, 261 and 493), wherein said antibody does not bind said signaling protein when not phosphorylated at said tyrosine.

47. An isolated phosphorylation site-specific antibody that specifically binds a human signaling protein selected from Column A of Table 1, Rows 248, 239, 244, 262, and 492 only when not phosphorylated at the tyrosine listed in corresponding Column D of Table 1, comprised within the phosphorylatable peptide sequence listed in corresponding Column E of Table 1 (SEQ ID NOs: 247, 238, 244, 261 and 493), wherein said antibody does not bind said signaling protein when phosphorylated at said tyrosine.

48. A method selected from the group consisting of:
(a) a method for detecting a human signaling protein selected from Column A of Table 1, Rows 248, 239, 244, 262, and 492 wherein said human signaling protein is phosphorylated at the tyrosine listed in corresponding Column D of Table 1, comprised within the phosphorylatable peptide sequence listed in corresponding Column E of Table 1 (SEQ ID NOs: 247, 238, 244, 261 and 493), comprising the step of adding an isolated phosphorylation-specific antibody according to claim 46, to a sample comprising said human signaling protein under conditions that permit the binding of said antibody to said human signaling protein, and detecting bound antibody;
(b) a method for quantifying the amount of a human signaling protein listed in Column A of Table 1, Rows 248, 239, 244, 262, and 492 that is phosphorylated at the corresponding tyrosine listed in Column D of Table 1, comprised within the phosphorylatable peptide sequence listed in corresponding Column E of Table 1 (SEQ ID NOs: 247, 238, 244, 261 and 493), in a sample using a heavy-isotope labeled peptide (AQUA™ peptide), said labeled peptide comprising a phosphorylated tyrosine at said corresponding lysine listed Column D of Table 1, comprised within the phosphorylatable peptide sequence listed in corresponding Column E of Table 1 as an internal standard; and
(c) a method comprising step (a) followed by step (b).

49. The method of claim 48, wherein said isolated phosphorylation-specific antibody is capable of specifically binding CK2-alpha2 only when phosphorylated at Y13, comprised within the phosphorylatable peptide sequence listed in Column E, Row 248, of Table 1 (SEQ ID NO: 247), wherein said antibody does not bind said protein when not phosphorylated at said tyrosine.

50. The method of claim 48, wherein said isolated phosphorylation-specific antibody is capable of specifically binding CK2-alpha2 only when not phosphorylated at Y13, comprised within the phosphorylatable peptide sequence listed in Column E, Row 248, of Table 1 (SEQ ID NO: 247), wherein said antibody does not bind said protein when phosphorylated at said tyrosine.

51. The method of claim 48, wherein said isolated phosphorylation-specific antibody is capable of specifically binding Bcr only when phosphorylated at Y279, comprised within the phosphorylatable peptide sequence listed in Column E, Row 239, of Table 1 (SEQ ID NO: 238), wherein said antibody does not bind said protein when not phosphorylated at said tyrosine.

52. The method of claim 48, wherein said isolated phosphorylation-specific antibody is capable of specifically binding Bcr only when not phosphorylated at Y279, comprised within the phosphorylatable peptide sequence listed in Column E, Row 239, of Table 1 (SEQ ID NO: 238), wherein said antibody does not bind said protein when phosphorylated at said tyrosine.

53. The method of claim 48, wherein said isolated phosphorylation-specific antibody is capable of specifically binding CDK3 only when phosphorylated at Y19, comprised within the phosphorylatable peptide sequence listed in Column E, Row 245, of Table 1 (SEQ ID NO: 244), wherein said antibody does not bind said protein when not phosphorylated at said tyrosine.

54. The method of claim 48, wherein said isolated phosphorylation-specific antibody is capable of specifically binding CDK3 only when not phosphorylated at Y19, comprised within the phosphorylatable peptide sequence listed in Column E, Row 245, of Table 1 (SEQ ID NO: 244), wherein said antibody does not bind said protein when phosphorylated at said tyrosine.

55. The method of claim 48, wherein said isolated phosphorylation-specific antibody is capable of specifically binding Fgr only when phosphorylated at Y208, comprised within the phosphorylatable peptide sequence listed in Column E, Row 262, of Table 1 (SEQ ID NO: 261), wherein said antibody does not bind said protein when not phosphorylated at said tyrosine.

56. The method of claim 48, wherein said isolated phosphorylation-specific antibody is capable of specifically binding Fgr only when not phosphorylated at Y208, comprised within the phosphorylatable peptide sequence listed in Column E, Row 262, of Table 1 (SEQ ID NO: 261), wherein said antibody does not bind said protein when phosphorylated at said tyrosine.

57. The method of claim 48, wherein said isolated phosphorylation-specific antibody is capable of specifically binding CLH-17 only when phosphorylated at Y1211, comprised within the phosphorylatable peptide sequence listed in Column E, Row 492, of Table 1 (SEQ ID NO: 493), wherein said antibody does not bind said protein when not phosphorylated at said tyrosine.

58. The method of claim 48, wherein said isolated phosphorylation-specific antibody is capable of specifically binding CLH-17 only when not phosphorylated at Y1211, comprised within the phosphorylatable peptide sequence listed in Column E, Row 492, of Table 1 (SEQ ID NO: 493), wherein said antibody does not bind said protein when phosphorylated at said tyrosine.
